SEQUENCE LISTING (42 sequences)

SEQ ID NO: 1 - DNA, 2391 bp, Artificial Sequence
  Construct
atttcatttatcaggtgttcagtgaatgcttactatgtaacagcacagttatcagcactggggaaatagatgagtaagataagatttgcactttcattagcttacatgccataaagagggaaataaagagaacaccagatgatgataagtttatgctgagaattaaaatgaagtgatgaaataatgggaatgtcaggtggctacttttggtgggatggtcaggaaaggcatctctggggagataaattttaagctcagacctgagtgaaaagaatgagccagccatggaaacattatgttaactcacatggtagtttgaaatgctttatctgatcaaaggtacttatttttggtgactttcaacaatattaagggtctataaaccaacactcatttgcataagaataactaccagtgaatctttttgtatgataggttttttgtttgttgtttttttgagacagagtctcgctctgtcgcccaggctggagtgcagtggcgcgatcttggctcactgcaacctctacctccccggttcaagtgattctcctgcctcagcctcccaaagtagctgggattacaggtgcctgccaccacgcctggctaatttttgtatttttagtagagatggggtttcaccgtgttgtccaggctcgtgtcaaacttctgacctcaagccatccacccgcctcggcctcccaaagtgctgggattacaggtgtgagccaccactcctggccatgataggttattttgtgatgaaaatacctacctcttaatttgtctgataaatttaaattttatgtctagatttcctaagatcagcacttccatattttaaagtaatctgtatcagactaactgctcttgcattcttttaataccagtgactactttgattcgtgaaacaatgtattttccttatgaatagtttttctcatggtgtatttattcttttaagttttgttttttaaatatacttcacttttgaatgtttcagacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataaaacttgtttattgcagcttataatggttacaaataaagcaatagcatcacaaatttcacaaataaagcatttttttcactgcattctagttgtggtttgtccaaactcatcaatgtatcttatcatgtctggatctccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacttcttgccctcggtcttcaggtcgttgcgcacggtctccaggctcatggtcacggcggcctgcagcatgtcctcctcgctcatggcgtcgcccaggtcgctgcccagggcgccgctgctggtggcggggcgctcgcaggggtggctgctctggccgctcaggtcgccctgctgctgctgctgctgctgctgctgctgcttctgctgctgtctgtaaatgaatgagaaaaccggtttagaaagtgcacagctgtcagggaagtcaacacttcagtgagcatgtgaccatgtggagtcagcttcctgtttcgtgctgcaatcgtaaggcctgctcaccattcatcatgttcgctaccttcacactttatctgacatacgagctccatgtgatttttgctttacattattcttcattccctctttaatcatattaagaatcttaagtaaatttgtaatctactaaatttccctggattaaggagcagttaccaaaagaaaaaaaaaaaaaaaagctagatgtggtggctcacatctgtaatcccagcactttgggaaaccaaggcaggagaggattgctagaacatttaatgaatactttaacataataatttaaacttcacagtaatttgtacagtctccaaaaattccttagacatcatggatatttttctttttttgagatggagtcttgctcttttaagctcagacctgagtgaaaagaatttgagacagagtctcgctctgtcgcctttcctaagatcagcacttccatatttggtgactttcaacaatattaagggtctataaaccaacactcatttgcataagaat

SEQ ID NO: 2 - DNA, 20 bp, Artificial Sequence
  gRNA target
aatatggaagtgctgatctt

SEQ ID NO: 3 - DNA, 1000 bp, Artificial Sequence
  Homology arm
atttcatttatcaggtgttcagtgaatgcttactatgtaacagcacagttatcagcactggggaaatagatgagtaagataagatttgcactttcattagcttacatgccataaagagggaaataaagagaacaccagatgatgataagtttatgctgagaattaaaatgaagtgatgaaataatgggaatgtcaggtggctacttttggtgggatggtcaggaaaggcatctctggggagataaattttaagctcagacctgagtgaaaagaatgagccagccatggaaacattatgttaactcacatggtagtttgaaatgctttatctgatcaaaggtacttatttttggtgactttcaacaatattaagggtctataaaccaacactcatttgcataagaataactaccagtgaatctttttgtatgataggttttttgtttgttgtttttttgagacagagtctcgctctgtcgcccaggctggagtgcagtggcgcgatcttggctcactgcaacctctacctccccggttcaagtgattctcctgcctcagcctcccaaagtagctgggattacaggtgcctgccaccacgcctggctaatttttgtatttttagtagagatggggtttcaccgtgttgtccaggctcgtgtcaaacttctgacctcaagccatccacccgcctcggcctcccaaagtgctgggattacaggtgtgagccaccactcctggccatgataggttattttgtgatgaaaatacctacctcttaatttgtctgataaatttaaattttatgtctagatttcctaagatcagcacttccatattttaaagtaatctgtatcagactaactgctcttgcattcttttaataccagtgactactttgattcgtgaaacaatgtattttccttatgaatagtttttctcatggtgtatttattcttttaagttttgttttttaaatatacttcacttttgaatgtttcag

SEQ ID NO: 4 - DNA, 214 bp, Artificial Sequence
  Coding sequence
acagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataa

SEQ ID NO: 5 - DNA, 135 bp, Artificial Sequence
  Terminator
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 6 - DNA, 208 bp, Artificial Sequence
  Terminator
tccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacag

SEQ ID NO: 7 - DNA, 214 bp, Artificial Sequence
  Coding sequence
ctacttcttgccctcggtcttcaggtcgttgcgcacggtctccaggctcatggtcacggcggcctgcagcatgtcctcctcgctcatggcgtcgcccaggtcgctgcccagggcgccgctgctggtggcggggcgctcgcaggggtggctgctctggccgctcaggtcgccctgctgctgctgctgctgctgctgctgctgcttctgctgctgt

SEQ ID NO: 8 - DNA, 110 bp, Artificial Sequence
  Splice acceptor
ctgtaaatgaatgagaaaaccggtttagaaagtgcacagctgtcagggaagtcaacacttcagtgagcatgtgaccatgtggagtcagcttcctgtttcgtgctgcaatc

SEQ ID NO: 9 - DNA, 374 bp, Artificial Sequence
  Right homology arm
gtaaggcctgctcaccattcatcatgttcgctaccttcacactttatctgacatacgagctccatgtgatttttgctttacattattcttcattccctctttaatcatattaagaatcttaagtaaatttgtaatctactaaatttccctggattaaggagcagttaccaaaagaaaaaaaaaaaaaaaagctagatgtggtggctcacatctgtaatcccagcactttgggaaaccaaggcaggagaggattgctagaacatttaatgaatactttaacataataatttaaacttcacagtaatttgtacagtctccaaaaattccttagacatcatggatatttttctttttttgagatggagtcttgctct

SEQ ID NO: 10 - DNA, 136 bp, Artificial Sequence
  Target site
tttaagctcagacctgagtgaaaagaatttgagacagagtctcgctctgtcgcctttcctaagatcagcacttccatatttggtgactttcaacaatattaagggtctataaaccaacactcatttgcataagaat

SEQ ID NO: 11 - DNA, 1205 bp, Artificial Sequence
  Construct
tttaagctcagacctgagtgaaaagaatttgagacagagtctcgctctgtcgcctttcctaagatcagcacttccatattttaaagtaatctgtatcagactaactgctcttgcattcttttaataccagtgactactttgattcgtgaaacaatgtattttccttatgaatagtttttctcatggtgtatttattcttttaagttttgttttttaaatatacttcacttttgaatgtttcagacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataaaacttgtttattgcagcttataatggttacaaataaagcaatagcatcacaaatttcacaaataaagcatttttttcactgcattctagttgtggtttgtccaaactcatcaatgtatcttatcatgtctggatctccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacttcttgccctcggtcttcaggtcgttgcgcacggtctccaggctcatggtcacggcggcctgcagcatgtcctcctcgctcatggcgtcgcccaggtcgctgcccagggcgccgctgctggtggcggggcgctcgcaggggtggctgctctggccgctcaggtcgccctgctgctgctgctgctgctgctgctgctgcttctgctgctgtctgtaaatgaatgagaaaaccggtttagaaagtgcacagctgtcagggaagtcaacacttcagtgagcatgtgaccatgtggagtcagcttcctgtttcgtgctgcaatctttaagctcagacctgagtgaaaagaatttgagacagagtctcgctctgtcgcctttcctaagatcagcacttccatattt

SEQ ID NO: 12 - DNA, 1723 bp, Artificial Sequence
  Construct
atttcatttatcaggtgttcagtgaatgcttactatgtaacagcacagttatcagcactggggaaatagatgagtaagataagatttgcactttcattagcttacatgccataaagagggaaataaagagaacaccagatgatgataagtttatgctgagaattaaaatgaagtgatgaaataatgggaatgtcaggtggctacttttggtgggatggtcaggaaaggcatctctggggagataaattttaagctcagacctgagtgaaaagaatgagccagccatggaaacattatgttaactcacatggtagtttgaaatgctttatctgatcaaaggtacttatttttggtgactttcaacaatattaagggtctataaaccaacactcatttgcataagaataactaccagtgaatctttttgtatgataggttttttgtttgttgtttttttgagacagagtctcgctctgtcgcccaggctggagtgcagtggcgcgatcttggctcactgcaacctctacctccccggttcaagtgattctcctgcctcagcctcccaaagtagctgggattacaggtgcctgccaccacgcctggctaatttttgtatttttagtagagatggggtttcaccgtgttgtccaggctcgtgtcaaacttctgacctcaagccatccacccgcctcggcctcccaaagtgctgggattacaggtgtgagccaccactcctggccatgataggttattttgtgatgaaaatacctacctcttaatttgtctgataaatttaaattttatgtctagaaatcctaagatcagcacttccatattttaaagtaatctgtatcagactaactgctcttgcattcttttaataccagtgactactttgattcgtgaaacaatgtattttccttatgaatagtttttctcatggtgtatttattcttttaagttttgttttttaaatatacttcacttttgaatgtttcagacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataaaacttgtttattgcagcttataatggttacaaataaagcaatagcatcacaaatttcacaaataaagcatttttttcactgcattctagttgtggtttgtccaaactcatcaatgtatcttatcatgtctggatcgtaaggcctgctcaccattcatcatgttcgctaccttcacactttatctgacatacgagctccatgtgatttttgctttacattattcttcattccctctttaatcatattaagaatcttaagtaaatttgtaatctactaaatttccctggattaaggagcagttaccaaaagaaaaaaaaaaaaaaaagctagatgtggtggctcacatctgtaatcccagcactttgggaaaccaaggcaggagaggattgctagaacatttaatgaatactttaacataataatttaaacttcacagtaatttgtacagtctccaaaaattccttagacatcatggatatttttctttttttgagatggagtcttgctct

SEQ ID NO: 13 - DNA, 19 bp, Artificial Sequence
  Primer
caaaggtgcccttgaggtt

SEQ ID NO: 14 - DNA, 20 bp, Artificial Sequence
  Primer
aggagaagtctgccgttact

SEQ ID NO: 15 - DNA, 22 bp, Artificial Sequence
  Primer
ggacaaaccacaactagaatgc

SEQ ID NO: 16 - DNA, 20 bp, Artificial Sequence
  Primer
taggaaaggacagtgggagt

SEQ ID NO: 17 - DNA, 24 bp, Artificial Sequence
  Primer
ccattatgtctcagttgttcagtg

SEQ ID NO: 18 - DNA, 19 bp, Artificial Sequence
  Primer
ccagaccatctcagacacc

SEQ ID NO: 19 - DNA, 18 bp, Artificial Sequence
  Primer
ggctgggcttccacttac

SEQ ID NO: 20 - DNA, 22 bp, Artificial Sequence
  Primer
gtggtttgtccaaactcatcaa

SEQ ID NO: 21 - DNA, 22 bp, Artificial Sequence
  Primer
agtaactctgcacttcccattg

SEQ ID NO: 22 - RNA, 19 nt, Artificial Sequence
  siRNA
cgucgguuguaggacuaaa

SEQ ID NO: 23 - RNA, 19 nt, Artificial Sequence
  shRNA
ggccugcauggauuccaug

SEQ ID NO: 24 - DNA, 27 bp, Artificial Sequence
  gRNA
gtgtgttactaattttataaatggagt

SEQ ID NO: 25 - DNA, 27 bp, Artificial Sequence
  gRNA
taattaaaaaaaaatgctaggcagaat

SEQ ID NO: 26 - DNA, 3332 bp, Artificial Sequence
  Construct
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttaggtcagtgaagagaagaacaaaaagcagcatattacagttagttgtcttcatcaatctttaaatatgttgtgtggtttttctctccctgtttccacagttgaggacccccagggcgacgccgcccagaagaccgacaccagccaccacgaccaggaccaccccaccttcaacaagatcacccccaacctggccgagttcgccttcagcctgtacaggcagctggcccaccagagcaacagcaccaacatcttcttcagccccgtgagcatcgccaccgccttcgccatgctgagcctgggcaccaaggccgacacccacgacgagatcctggagggcctgaacttcaacctgaccgagatccccgaggcccagatccacgagggcttccaggagctgctgaggaccctgaaccagcccgacagccagctgcagctgaccaccggcaacggcctgttcctgagcgagggcctgaagctggtggacaagttcctggaggacgtgaagaagctgtaccacagcgaggccttcaccgtgaacttcggcgacaccgaggaggccaagaagcagatcaacgactacgtggagaagggcacccagggcaagatcgtggacctggtgaaggagctggacagggacaccgtgttcgccctggtgaactacatcttcttcaagggcaagtgggagaggcccttcgaggtgaaggacaccgaggaggaggacttccacgtggaccaggtgaccaccgtgaaggtgcccatgatgaaacgcctcggaatgttcaacatccagcactgcaagaagctgagcagctgggtgctgctgatgaagtacctgggcaacgccaccgccatcttcttcctgcccgacgagggcaagctgcagcacctggagaacgagctgacccacgacatcatcaccaagttcctggagaacgaggacaggaggagcgccagcctgcacctgcccaagctgagcatcaccggcacctacgacctgaagagcgtgctgggccagctgggcatcaccaaggtgttcagcaacggcgccgacctgagcggcgtgaccgaggaggcccccctgaagctgagcaaggccgtgcacaaggccgtgctgaccatcgacgagaagggcaccgaggccgccggcgccatgttcctggaggccatccccatgagcatcccccccgaggtgaagttcaacaagcccttcgtgttcctgatgatcgagcagaacaccaagagccccctgttcatgggcaaggtggtgaaccccacccagaagtaacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttgaacattccgaggcgtttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcaactgaaatgtaaaagaataattctttagttttagcaaaaaagaaaacatcatgaaaattttacatctcttaagaaagtctttgtttttaatccaaataatcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 27 - RNA, 21 nt, Artificial Sequence
  shRNA
gaugaagcguuuaggcauguu

SEQ ID NO: 28 - RNA, 21 nt, Artificial Sequence
  shRNA
gaacucacccacgauaucauu

SEQ ID NO: 29 - RNA, 21 nt, Artificial Sequence
  shRNA
ccuaugaucugaagagcguuu

SEQ ID NO: 30 - RNA, 21 nt, Artificial Sequence
  shRNA
ugauaucgugggugaguucuu

SEQ ID NO: 31 - RNA, 21 nt, Artificial Sequence
  shRNA
caugccuaaacgcuucaucuu

SEQ ID NO: 32 - RNA, 21 nt, Artificial Sequence
  shRNA
acgcucuucagaucauagguu

SEQ ID NO: 33 - DNA, 27 bp, Artificial Sequence
  gRNA
ttcaactgtatccaacgtaatttgagt

SEQ ID NO: 34 - DNA, 27 bp, Artificial Sequence
  gRNA
ctcaaattacgttggatacagttgaat

SEQ ID NO: 35 - DNA, 27 bp, Artificial Sequence
  gRNA
acatgacagaaacactaaatcttgagt

SEQ ID NO: 36 - DNA, 27 bp, Artificial Sequence
  gRNA
gatcgggaactggcatcttcagggagt

SEQ ID NO: 37 - DNA, 27 bp, Artificial Sequence
  gRNA
ggcagaatgactcaaattacgttggat

SEQ ID NO: 38 - DNA, 27 bp, Artificial Sequence
  gRNA
gtatctttgatgacaataatgggggat

SEQ ID NO: 39 - DNA, 27 bp, Artificial Sequence
  gRNA
cagaaacactaaatcttgagtttgaat

SEQ ID NO: 40 - DNA, 27 bp, Artificial Sequence
  gRNA
atatcaataatataaccacctaagggt

SEQ ID NO: 41 - DNA, 27 bp, Artificial Sequence
  gRNA
atgcacagatataaacacttaacgggt

SEQ ID NO: 42 - DNA, 27 bp, Artificial Sequence
  gRNA
tgttggtgaaaaaatataactttgagt

(19) United States
(12) Patent Application Publication
(10) Pub. No.: US 2023/0190962 A1
(43) Pub. Date: Jun. 22, 2023

(54) METHODS FOR TREATING GAIN-OF-FUNCTION DISORDERS COMBINING GENE EDITING AND GENE THERAPY

(71) Applicant: BLUEALLELE CORPORATION, Oakdale, MN (US)

(72) Inventor: Nicholas BALTES, Oakdale, MN (US)

(21) Appl. No.: 17/802,678

(22) PCT Filed: Feb. 28, 2021
(86) PCT No.: PCT/US21/20158

§ 371 Date: Aug. 26, 2022

Related U.S. Application Data
(60) Provisional application No. 62/983,048, filed on Feb. 28, 2020.

(51) Int. Cl. A61K 48/00; C12N 15/11; C12N 9/22; C12N 15/90; C12N 15/113

(52) U.S. Cl. CPC A61K 48/0066; C12N 15/11; C12N 9/22; C12N 15/907; C12N 15/113; A61K 48/0075; A61K 48/0083; C12N 2310/20; C12N 2800/80; C12N 2310/531; C12N 2310/14

(57) ABSTRACT

Methods and compositions for modifying the expression of endogenous genes or modifying the coding sequence of endogenous genes.

### REFERENCE TO RELATED APPLICATIONS

The application claims the benefit of previously filed and co-pending U.S. Provisional Patent Application No. 62/983,048, filed Feb. 28, 2020, which is hereby incorporated herein by reference in its entirety.

### SEQUENCE LISTING

The instant application contains a Sequence Listing which has been submitted in ASCII format via EFS-Web and is hereby incorporated herein by reference in its entirety. Said ASCII copy, created on Feb. 28, 2020, is named Sequence_Listing_1026007PCT.txt and is 21,734 bytes is size.

### TECHNICAL FIELD

The present document is in the field of genome editing and gene therapy. More specifically, this document relates to the targeted modification of endogenous genes, or reduction of endogenous gene expression along with gene expression from an integrated transgene.

### BACKGROUND

Monogenic disorders are caused by one or more mutations in a single gene, examples of which include sickle cell disease (hemoglobin-beta gene), cystic fibrosis (cystic fibrosis transmembrane conductance regulator gene), and Tay-Sachs disease (beta-hexosaminidase A gene). Monogenic disorders have been an interest for gene therapy, as replacement of the defective gene with a functional copy could provide therapeutic benefits. However, one bottleneck for generating effective therapies includes the size of the functional copy of the gene. Many delivery methods, including those that use viruses, have size limitations which hinder the delivery of large transgenes. Further, many genes have alternative splicing patterns resulting in a single gene coding for multiple proteins. Methods to correct regions of a defective gene may provide additional means to treat monogenic disorders.

### SUMMARY

Gene editing holds promise for correcting mutations found in genetic disorders; however, many challenges remain for creating effective therapies for individual disorders, including those that are caused by gain-of-function mutations, or where precise repair is required. These challenges are seen with disorders such as spinocerebellar ataxia type 3, spinocerebellar ataxia type 6, and autosomal dominant Parkinson's disease, wherein the disorder is caused by gain-of-function mutations.

In one aspect, the methods described herein provide novel approaches for correcting gene expression from gain-of-function disorders. Frequently, autosomal dominant diseases result from a mutation in one allele of a gene comprising two or more alleles. The allele having the mutation then produces a product (i.e., mRNA and/or protein) that possesses a new molecular function. To treat gain-of-function mutations, it would be desirable to remove the gene products produced by the mutant allele while preserving the production of normal WT protein. This application describes novel ways to correct gain-of-function mutations using gene editing. The gene editing described herein results in correction of the phenotype, regardless of what allele the editing occurs within. A silencing agent is delivered that down-regulates expression from the endogenous alleles of the gene. That is, the endogenous mutant allele is turned down and any endogenous WT allele(s) are turned down. The transgene is refractory to the silencing agent and the phenotype can be corrected through the coding sequence provided within the transgenes. The transgene can be integrated into either the mutant or the WT allele. In either case, the result will be a reduced or eliminated production of products from the mutant allele, while retaining WT protein production. In additional applications, the transgene may be integrated into a different gene that is not targeted by the silencing agent, but one that has a promoter-of-interest.

In more embodiments, the methods described herein can be used to modify the expression of an endogenous gene in a cell comprising a first allele of the endogenous gene and a second allele of the endogenous gene, where the methods include administering a transgene to the cell, wherein the transgene comprises a first coding sequence, integrating the transgene into the first allele of the endogenous gene to create a modified first allele, and administering a silencing agent to the cell that reduces expression of the endogenous gene, wherein the transgene comprises a coding sequence that is not silenced by the silencing agent, and wherein the modified first allele is expressed at a higher level than the second allele.

The methods can also include modifying expression of a first endogenous gene in a cell comprising two or more alleles, the two or more alleles comprising at least a first and second allele, where the methods include administering a transgene to the cell, wherein the transgene comprises a first coding sequence, integrating the transgene into a second endogenous gene, administering a silencing agent to the cell that reduces expression of the first endogenous gene, wherein the transgene comprises a coding sequence that is not silenced by the silencing agent, and wherein the modified first allele is expressed at a higher level than the non-modified alleles.

In more embodiments, the methods described herein combine gene editing and gene silencing to correct and silence, respectively, the mRNA and protein produced by an endogenous gene comprising a gain-of-function mutation. The methods include i) integrating a coding sequence into one or more alleles of an endogenous gene comprising a gain-of-function mutation, wherein the coding sequence comprises sequence that is designed to correct the gain-of-function mutation and is resistant to a user-generated RNA interference agent, and ii) afterward, or concurrently, delivering an RNA interference agent that reduces the expression of the wild type, mutant, or un-modified alleles. The methods described herein can be used to correct gain-of-function mutations caused by gene multiplications (e.g., SNCA and Parkinson disease) or mutations or deletions or insertions (e.g., trinucleotide repeat expansions). The methods can be used on genes that produce one or more isoforms. In some embodiments, rare-cutting endonucleases can be used to integrate a transgene comprising a corrective coding sequence resistant to silencing. The transgene can be integrated into one or more alleles of the endogenous gene comprising a gain-of-function mutation. The transgene can comprise a full or partial corrective coding sequence. If the transgene comprises a partial coding sequence, the transgene can further comprise a splice acceptor or splice donor operably linked to the partial coding sequence. The transgene can further comprise a terminator operably linked to the corrective coding sequence resistant to silencing (if targeting the 3′ region of a gene for correction) or a promoter operably linked to the silencing-resistant coding sequence (if targeting the 5′ region of a gene). The gain-of-function mutation can be a mutation that results in a disease selected from the group consisting of AATD (alpha-1 antitrypsin deficiency), HD (Huntington's Disease), SBMA (Spinobulbar Muscular Atrophy), SCA1 (Spinocerebellar Ataxia Type 1), SCA2 (Spinocerebellar Ataxia Type 2), SCA3 (Spinocerebellar Ataxia Type 3 or Machado-Joseph Disease), SCA6 (Spinocerebellar Ataxia Type 6), SCAT (Spinocerebellar Ataxia Type 7), Fragile X Syndrome, Fragile XE Mental Retardation, Friedreich's Ataxia, Myotonic Dystrophy type 1, Myotonic Dystrophy type 2, Spinocerebellar Ataxia Type 8, Spinocerebellar Ataxia Type 12, spinal and bulbar muscular atrophy, JPH3, Amyotrophic Lateral Sclerosis (ALS), hereditary motor and sensory neuropathy type IIC, postsynaptic slow-channel congenital myasthenic syndrome, PRPS1 superactivity, Parkinson disease, tubular aggregate myopathy, achondroplasia, lubs X-linked mental retardation syndrome, and autosomal dominant retinitis pigmentosa.

In another aspect, the methods described herein provide approaches for integrating transgenes into endogenous genes. The methods are based in part on the design of bidirectional transgenes compatible with integration through multiple repair pathways. The transgenes described herein can be integrated into genes by the homologous recombination pathway, the non-homologous end joining pathway, or both the homologous recombination and non-homologous end joining pathway. Further, the outcome of integration in any case (HR, NHEJ forward, NHEJ reverse) can result in precise correction/alteration of the target gene's protein product. The transgenes described herein can be used to fix or introduce mutations in the 5′ or 3′ region of genes-of-interest. The methods described herein are particularly useful in cases where retaining the endogenous promoter is necessary, retaining isoform production is necessary, precise editing is necessary, or where the coding sequence of the target gene exceeds the size capacity of standard vectors or viral vectors. The methods described herein can be used for applied research (e.g., therapeutic development) or basic research (e.g., creation of animal models, or understanding gene function).

In another aspect, the methods describe administering a silencing agent that reduces expression of an endogenous gene at the same time or following integration of the corrective coding sequence resistant to silencing. The agent can include antisense oligonucleotides, shRNA, siRNA, miRNA, or any other RNA suitable for reducing gene expression. The use of the silencing agent further reduces the expression of the gain-of-function allele, particularly in cells that do not have an integration event or have a single integration event but within a wild-type allele.

The methods described herein are compatible with current in vivo delivery vehicles including adeno-associated virus vectors and lipid nanoparticles, and they address several challenges with achieving precise alteration of gene products, particularly those with gain-of-function mutations and those that produce multiple isoforms.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DESCRIPTION OF DRAWINGS

FIG. 1 is an illustration of a method for modifying expression of a gene with a gain-of-function mutation using a partial coding sequence resistant to silencing. The method includes identifying a gene comprising a gain-of-function mutation (step 1), design of a silencing agent for reducing the mRNA or protein levels of the gene target (step 2), design of a donor molecule with corrective sequence that is resistant to silencing (step 3), and integrating the donor molecule into at least one allele of the gene target, including the WT allele (step 4.1) or the gain-of-function allele (step 4.2). The method also includes transient delivery of the silencing agent to reduce expression of un-modified alleles. ASO, antisense oligonucleotide; GOF, gain-of-function.

FIG. 2 is an illustration of a method for modifying expression of a gene with a gain-of-function mutation using a partial coding sequence resistant to silencing. The method includes identifying a gene comprising a gain-of-function mutation (step 1), design of a silencing agent for reducing the mRNA or protein levels of the gene target, wherein the silencing agent targets mRNA sequence produced from exon 3 (step 2), design of a donor molecule with corrective sequence that is resistant to silencing (step 3), and integrating the donor molecule into at least one allele of the gene target, including the WT allele (step 4.1) or the gain-of-function allele (step 4.2). The method also includes transient delivery of the silencing agent to reduce expression of un-modified alleles. ASO, antisense oligonucleotide; GOF, gain-of-function; CDS, coding sequence.

FIG. 3 presents illustrations of transgenes comprising corrective coding sequence resistant to silencing and target sites for integration. In some embodiments, the transgene comprises a splice acceptor operably linked to a coding sequence resistant to silencing which is operably linked to a terminator. The transgene is flanked with additional sequences (AS1 and AS2) that facilitate integration into a target gene. In another embodiment, the transgene comprises a first and second splice acceptor operably linked to a first and second coding sequence resistant to silencing which are operably linked to a first and second terminator. The coding sequences are in a tail-to-tail orientation and flanked by additional sequences. The additional sequences can be ITRs from AAV, nuclease cleavage sites, homology arms, or exposed DNA ends. The transgenes can be integrated within introns of the endogenous gene, or at the intron-exon junctions. AS, additional sequence; SA, splice acceptor; CDS, coding sequence; T, terminator; UTR, untranslated region.

FIG. 4 presents illustrations of transgenes comprising corrective coding sequence resistant to silencing and target sites for integration. In some embodiments, the transgene comprises a splice acceptor operably linked to a 2A sequence operably linked to a coding sequence resistant to silencing which is operably linked to a terminator. The transgene is flanked with additional sequences (AS1 and AS2) that facilitate integration into a target gene. In another embodiment, the transgene comprises a first and second splice acceptor operably linked to first and second 2A sequence operably linked to a first and second coding sequence resistant to silencing which are operably linked to a first and second terminator. The coding sequences are in a tail-to-tail orientation and flanked by additional sequences. The additional sequences can be ITRs from AAV, nuclease cleavage sites, homology arms, or exposed DNA ends. The transgenes can be integrated within introns of the endogenous gene, or at the intron-exon junctions. AS, additional sequence; SA, splice acceptor; CDS, coding sequence; T, terminator; UTR, untranslated region.

FIG. 5 presents illustrations of transgenes comprising corrective coding sequence resistant to silencing and target sites for integration. In some embodiments, the transgene comprises a coding sequence resistant to silencing operably linked to a terminator. The transgene is flanked with additional sequences (AS1 and AS2) that facilitate integration into a target gene. In another embodiment, the transgene comprises a first and second coding sequence resistant to silencing which are operably linked to a first and second terminator. The coding sequences are in a tail-to-tail orientation and flanked by additional sequences. The additional sequences can be ITRs from AAV, nuclease cleavage sites, homology arms, or exposed DNA ends. The transgenes can be integrated within the 5′ UTR of the target gene. AS, additional sequence; CDS, coding sequence; T, terminator; UTR, untranslated region.

FIG. 6 presents illustrations of transgenes comprising corrective coding sequence resistant to silencing and target sites for integration. In some embodiments, the transgene comprises a promoter operably linked to a coding sequence resistant to silencing which is operably linked to a splice donor. The transgene is flanked with additional sequences (AS1 and AS2) that facilitate integration into a target gene. In another embodiment, the transgene comprises a first and second promoter operably linked to a first and second coding sequence resistant to silencing which are operably linked to a first and second splice donor. The coding sequences are in a head-to-head orientation and flanked by additional sequences. The additional sequences can be ITRs from AAV, nuclease cleavage sites, homology arms, or exposed DNA ends. The transgenes can be integrated within the gene in either introns or coding exons, but before the endogenous splice acceptors. AS, additional sequence; SD, splice donor; CDS, coding sequence; P, promoter; UTR, untranslated region.

FIG. 7 is an illustration of a transgene comprising corrective coding sequence resistant to silencing and target sites for integration. The transgene comprises a splice acceptor operably linked to a 2A sequence operably linked to a coding sequence resistant to silencing operably linked to a splice donor. The transgene is flanked with additional sequences (AS1 and AS2) that facilitate integration into a target gene. The additional sequences can be ITRs from AAV, nuclease cleavage sites, homology arms, or exposed DNA ends. The transgenes can be integrated within introns of the endogenous gene. AS, additional sequence; CDS, coding sequence; T, terminator; UTR, untranslated region.

FIG. 8 is an illustration of exon 9, intron 9, exon 10, intron 10 and exon 11 of the ATXN3 gene. Also shown is the pBA1012-D1 transgene for integration in the ATXN3 gene.

FIG. 9 are images of gels detecting integration of transgenes into the ATXN3 gene. 1, 100 bp ladder with top band running at 1,517 bp; 2, pBA1135 5′ junction; 3, pBA1136 5′ junction; 4, pBA1137 5′ junction; 5, pBA1135 3′ junction; 6, pBA1136 3′ junction; 7, pBA1137 3′ junction; 8, 1kb ladder with darker bands running at 500 bp, 1,000 bp and 3,000 bp; 9, 1kb ladder with darker bands running at 500 bp, 1,000 bp and 3,000 bp; 10, pBA1135 inverted 5′ junction; 11, 1kb ladder with darker bands running at 500 bp, 1,000 bp and 3,000 bp; 12, pBA1136 inverted 5′ junction; 13, 1kb ladder with darker bands running at 500 bp, 1,000 bp and 3,000 bp; 14;, primer pair oNJB156+oNJB113; 15, primer pair 114+162; 16, primer pair oNJB116+oNJB113; 17, primer pair oNJB114+oNJB170; 18, primer pair oNJB167+oNJB170; 19, 100 bp ladder with the dark band running at 500 bp; 20, genomic DNA from transfection with pBA1135 and nuclease; 21, genomic DNA from transfection with pBA1136 and nuclease; 22, genomic DNA from transfection with pBA1137 and nuclease; 23, genomic DNA from transfection with water; 24, no DNA control.

FIG. 10 is an illustration of exon 9, intron 9, exon 10, intron 10 and exon 11 and the 3′ UTR of the ATXN3 gene. Also shown is a bidirectional transgene for integration into intron 9, and the miRNA target site within the 3′ UTR. 1, target site for integration of the transgene; 2, target site for miRNA binding in the corresponding mRNA transcript.

FIG. 11 is an illustration of the SOD1 gene. Also shown are bidirectional templates for integration into 5′ UTR (1), intron 1 (2) or intron 3 (3), and the shRNA targeting SOD1 mRNA.

FIG. 12 is an illustration of the albumin gene. Also shown are bidirectional templates for integration into intron 1 (1) or intron 13 (2, 3).

### DETAILED DESCRIPTION

Disclosed herein are methods and compositions for modifying the expression of endogenous genes. In some embodiments, the methods include inserting a transgene into an endogenous gene, wherein the transgene provides a coding sequence which substitutes for the endogenous gene's coding sequence. Also disclosed are methods for delivering an agent for reducing the expression of remaining unmodified endogenous genes.

This disclosure provides a method for modifying the expression of an endogenous gene with a first and second allele, the method comprising administering a transgene, wherein the transgene comprises a first coding sequence, integrating the transgene into the first allele of the endogenous gene to create a modified allele, and administering a silencing agent that reduces expression of the second allele of the endogenous gene, but not the modified allele. The method can include having the first coding sequence operably linked to a first splice acceptor sequence. The method can include having the first coding sequence operably linked to a terminator. The method can include having the first coding sequence comprising nucleotide differences compared to the corresponding wild type sequence that prevents silencing by the silencing agent. The mutations can be substitutions or deletions of the corresponding target site within the first coding sequence. The method can include designing a transgene with a second coding sequence and second splice acceptor and second terminator. The first and second coding sequences can be operably linked to the first and second splice acceptor sequences. The first and second coding sequences can be operably linked to the first and second terminator, or one bidirectional terminator. The first and second coding sequences can be positioned adjacent to each other in a tail-to-tail orientation. The first and second coding sequences can comprise nucleotide differences compared to the corresponding wild type sequence that prevents silencing by the silencing agent. The first and second coding sequences can encode a reporter gene, a purification tag, a partial protein, a full protein, or amino acids that are homologous to amino acids encoded by the endogenous gene. The first and second coding sequences can encode the same amino acids. The first and second coding sequences can encode the same amino acid sequence but differ in nucleic acid sequence. The transgene may be provided on a viral vector. The viral vector can be selected from an adenovirus vector, an adeno-associated virus vector, or a lentivirus vector. The transgene can be 4.7kb or less in size. The transgene may be provided on a non-viral vector. The transgene can be integrated into an endogenous gene using a rare-cutting endonuclease, including a CRISPR nuclease, a TAL effector nuclease, a zinc-finger nuclease, or a meganuclease. The nuclease can be targeted to a sequence within an intron of the endogenous gene. The endogenous gene can include SERPINA, SOD1, TRPV4, CHRNA1, CHRND, CHRNE, CHRNB 1, PRPS1, LRRK2, STIM1, FGFR3, MECP2, SNCA, ATXN1, ATXN2, ATXN3, CACNA1A, ATXN7, TBP, HTT, AR, FXN, DMPK, PABPN1, ATXN8, RHO, TTR or C9orf72. The silencing agent can be a DNA oligonucleotide agent or an RNA oligonucleotide agent. The DNA oligonucleotide agent can be antisense oligonucleotides. The RNA oligonucleotide agent can be microRNA, short hairpin RNA, double-stranded RNA, or short interfering RNA. The oligonucleotide agent can be delivered on a second transgene, or as RNA or DNA. The oligonucleotide agent does not need to be present within the transgene, and can be delivered concurrent with the transgene, or after the transgene is delivered.

This disclosure also provides a method for modifying the expression of an endogenous gene having two or more alleles, the two or more alleles comprising at least a first and second allele, the method comprising administering a transgene, wherein the transgene comprises a first coding sequence, integrating the transgene into at least the first allele, and administering a silencing agent that reduces expression of at least the second allele, but not the first allele. The method can include having the first coding sequence operably linked to a first splice acceptor sequence. The method can include having the first coding sequence operably linked to a terminator. The method can include having the first coding sequence comprising nucleotide differences compared to the corresponding wild type sequence that prevents silencing by the silencing agent. The mutations can be substitutions or deletions of the corresponding target site within the first coding sequence. The method can include designing a transgene with a second coding sequence and second splice acceptor and second terminator. The first and second coding sequences can be operably linked to the first and second splice acceptor sequences. The first and second coding sequences can be operably linked to the first and second terminator, or one bidirectional terminator. The first and second coding sequences can be positioned adjacent to each other in a tail-to-tail orientation. The first and second coding sequences can comprise nucleotide differences compared to the corresponding wild type sequence that prevents silencing by the silencing agent. The first and second coding sequences can encode a reporter gene, a purification tag, a partial protein, a full protein, or amino acids that are homologous to amino acids encoded by the endogenous gene. The first and second coding sequences can encode the same amino acids. The first and second coding sequences can encode the same amino acid sequence but differ in nucleic acid sequence. The transgene may be provided on a viral vector. The viral vector can be selected from an adenovirus vector, an adeno-associated virus vector, or a lentivirus vector. The transgene can be 4.7kb or less in size. The transgene may be provided on a non-viral vector. The transgene can be integrated into an endogenous gene using a rare-cutting endonuclease, including a CRISPR nuclease, a TAL effector nuclease, a zinc-finger nuclease, or a meganuclease. The nuclease can be targeted to a sequence within an intron of the endogenous gene. The endogenous gene can include SERPINA, SOD1, TRPV4, CHRNA1, CHRND, CHRNE, CHRNB 1, PRPS1, LRRK2, STIM1, FGFR3, MECP2, SNCA, ATXN1, ATXN2, ATXN3, CACNA1A, ATXN7, TBP, HTT, AR, FXN, DMPK, PABPN1, ATXN8, RHO, TTR or C9orf72. The silencing agent can be a DNA oligonucleotide agent or an RNA oligonucleotide agent. The DNA oligonucleotide agent can be antisense oligonucleotides. The RNA oligonucleotide agent can be microRNA, short hairpin RNA, double-stranded RNA, or short interfering RNA. The oligonucleotide agent can be delivered on a second transgene, or as RNA or DNA. The oligonucleotide agent does not need to be present within the transgene, and can be delivered concurrent with the transgene, or after the transgene is delivered.

This document also features a method of modifying the expression of a first endogenous gene with two alleles, the method comprising administering a transgene, wherein the transgene comprises a first coding sequence, wherein the coding sequence encodes amino acids with homology to the first endogenous gene, integrating the transgene into a second endogenous gene, and administering a silencing agent that reduces expression of the first endogenous gene.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.),

Academic Press, San Diego, 1999; and METHODS IN MOLECULAR BIOLOGY, Vol. 119, “Chromatin Protocols” (P. B. Becker, ed.) Humana Press, Totowa, 1999.

As used herein, the terms “nucleic acid” and “polynucleotide,” can be used interchangeably. Nucleic acid and polynucleotide can refer to a deoxyribonucleotide or ribonucleotide polymer, in linear or circular conformation, and in either single- or double-stranded form. These terms are not to be construed as limiting with respect to the length of a polymer. The terms can encompass known analogues of natural nucleotides, as well as nucleotides that are modified in the base, sugar and/or phosphate moieties.

The terms “polypeptide,” “peptide” and “protein” can be used interchangeably to refer to amino acid residues covalently linked together. The term also applies to proteins in which one or more amino acids are chemical analogues or modified derivatives of corresponding naturally-occurring amino acids.

The terms “operatively linked” or “operably linked” are used interchangeably and refer to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

As used herein, the term “cleavage” refers to the breakage of the covalent backbone of a nucleic acid molecule. Cleavage can be initiated by a variety of methods including, but not limited to, enzymatic or chemical hydrolysis of a phosphodiester bond. Cleavage can refer to both a single-stranded nick and a double-stranded break. A double-stranded break can occur as a result of two distinct single-stranded nicks. Nucleic acid cleavage can result in the production of either blunt ends or staggered ends. In certain embodiments, rare-cutting endonucleases are used for targeted double-stranded or single-stranded DNA cleavage.

An “exogenous” molecule can refer to a small molecule (e.g., sugars, lipids, amino acids, fatty acids, phenolic compounds, alkaloids), or a macromolecule (e.g., protein, nucleic acid, carbohydrate, lipid, glycoprotein, lipoprotein, polysaccharide), or any modified derivative of the above molecules, or any complex comprising one or more of the above molecules, generated or present outside of a cell, or not normally present in a cell. Exogenous molecules can be introduced into cells. Methods for the introduction of exogenous molecules into cells can include lipid-mediated transfer, electroporation, direct injection, cell fusion, particle bombardment, calcium phosphate co-precipitation, DEAE-dextran-mediated transfer and viral vector-mediated transfer.

An “endogenous” molecule is a small molecule or macromolecule that is present in a particular cell at a particular developmental stage under particular environmental conditions. An endogenous molecule can be a nucleic acid, a chromosome, the genome of a mitochondrion, chloroplast or other organelle, or a naturally-occurring episomal nucleic acid. Additional endogenous molecules can include proteins, for example, transcription factors and enzymes.

As used herein, a “gene,” refers to a DNA region that encodes a gene product, including all DNA regions which regulate the production of the gene product. Accordingly, a gene includes, but is not necessarily limited to, coding sequences, intron sequences, exon sequences, promoter sequences, terminators, translational regulatory sequences such as ribosome binding sites and internal ribosome entry sites, enhancers, silencers, insulators, boundary elements, replication origins, matrix attachment sites and locus control regions.

An “endogenous gene” refers to a gene that is normally present in the genome of a particular cell.

A cell typically comprises two alleles of each autosomal gene, one on each chromosome. The two alleles are referred to herein as a “first allele” and a “second allele.” In genetic disorders that result from gene duplication, a cell may comprise more than two alleles of an endogenous gene.

“Gene expression” refers to the conversion of the information, contained in a gene, into a gene product. A gene product can be the direct transcriptional product of a gene. For example, the gene product can be, but not limited to, mRNA, tRNA, rRNA, antisense RNA, ribozyme, structural RNA, or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Encoding” refers to the conversion of the information contained in a nucleic acid, into a product, wherein the product can result from the direct transcriptional product of a nucleic acid sequence. For example, the product can be, but not limited to, mRNA, tRNA, rRNA, antisense RNA, ribozyme, structural RNA, or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

A “target site” or “target sequence” is a nucleic acid sequence to which a binding molecule will bind, provided sufficient conditions for binding exist, such as an endonuclease, including for example a rare-cutting. The target site can be an endogenous gene which may be native to the cell or heterologous.

As used herein, the term “recombination” refers to a process of exchange of genetic information between two polynucleotides. The term “homologous recombination (HR)” refers to a specialized form of recombination that can take place, for example, during the repair of double-strand breaks. Homologous recombination requires nucleotide sequence homology present on a “donor” molecule. The donor molecule can be used by the cell as a template for repair of a double-strand break. Information within the donor molecule that differs from the genomic sequence at or near the double-strand break can be stably incorporated into the cell's genomic DNA.

The term “homologous” as used herein refers to a sequence of nucleic acids or amino acids having similarity to a second sequence of nucleic acids or amino acids. In some embodiments, a the homologous sequences can have at least 80% sequence identity (e.g., 81%, 85%, 90%, 95%, 96%, 97%, 98%, or 99% sequence identity) to one another.

A “target site” or “target sequence” defines a portion of a nucleic acid to which a rare-cutting endonuclease will bind, provided sufficient conditions for binding exist.

The term “transgene” as used herein refers to a sequence of nucleic acids that can be transferred to an organism or cell. The transgene may comprise a gene or sequence of nucleic acids not normally present in the target organism or cell. Additionally, the transgene may comprise a copy of a gene or sequence of nucleic acids that is normally present in the target organism or cell. A transgene can be an exogenous DNA sequence introduced into the cytoplasm or nucleus of a target cell. In some embodiments, the transgenes described herein contain coding sequences, wherein the coding sequences encodes a full-length protein, or a portion of a protein normally produced by an endogenous gene in the host cell.

As used herein, the term “pathogenic” refers to anything that can cause disease. A pathogenic mutation can refer to a modification in a gene which causes disease. A pathogenic gene refers to a gene comprising a modification which causes disease. By means of example, a pathogenic ATXN2 gene in patients with spinocerebellar ataxia 2 refers to an ATXN2 gene with an expanded CAG trinucleotide repeat, wherein the expanded CAG trinucleotide repeat causes the disease.

As used herein, the term “tail-to-tail” refers to an orientation of two units in opposite and reverse directions. The two units can be two sequences on a single nucleic acid molecule, where the 3′ end of each sequence are placed adjacent to each other. For example, a first nucleic acid having the elements, in a 5′ to 3′ direction, [splice acceptor 1]—[partial coding sequence 1]—[terminator 1] and a second nucleic acid having the elements [splice acceptor 2]—[partial coding sequence 2]—[terminator 2] can be placed in tail-to-tail orientation resulting in [splice acceptor 1]—[partial coding sequence 1]—[terminator 1]—[terminator 2 RC]—[partial coding sequence 2 RC] - [splice acceptor 2 RC], where RC refers to reverse complement.

As used herein, the term “head-to-head” refers to an orientation of two units in opposite and reverse directions. The two units can be two sequences on a single nucleic acid molecule, where the 5′ end of each sequence are placed adjacent to each other. For example, a first nucleic acid having the elements, in a 5′ to 3′ direction, [promoter 1]—[partial coding sequence 1]—[splice donor 1] and a second nucleic acid having the elements [promoter 2]—[partial coding sequence 2]—[splice donor 2] can be placed in head-to-head orientation resulting in [splice donor 1 RC]—[partial coding sequence 1 RC]—[promoter 1 RC]—[promoter 2]—[partial coding sequence 2]—[splice donor 2] where RC refers to reverse complement.

The term “integrating” as used herein refers to the process of adding DNA to a target region of DNA. As described herein, integration can be facilitated by several different means, including non-homologous end joining, homologous recombination, or targeted transposition. By way of example, integration of a user-supplied DNA molecule into a target gene can be facilitated by non-homologous end joining. Here, a targeted-double strand break is made within the target gene and a user-supplied DNA molecule is administered. The user-supplied DNA molecule can comprise exposed DNA ends to facilitate capture during repair of the target gene by non-homologous end joining. The exposed ends can be present on the DNA molecule upon administration (i.e., administration of a linear DNA molecule) or created upon administration to the cell (i.e., a rare-cutting endonuclease cleaves the user-supplied DNA molecule within the cell to expose the ends). Additionally, the user-supplied DNA molecule may be provided on a viral vector, including an adeno-associated virus vector. In another example, integration occurs though homologous recombination. Here, the user-supplied DNA can have a left and right homology arm.

The term “intron-exon junction” refers to a specific location within a gene. The specific location is between the last nucleotide in an intron and the first nucleotide of the following exon. When integrating a transgene described herein, the transgene can be integrated within the “intron-exon junction.” If the transgene comprises cargo, the cargo will be integrated immediately following the last nucleotide in the intron. In some cases, integrating a transgene within the intron-exon junction can result in removal of sequence within the exon (e.g., integration via HR and replacement of sequence within the exon with the cargo within the transgene).

The term “exon-intron junction” refers to a specific location within a gene. The specific location is between the last nucleotide in an exon and the first nucleotide of the following intron. When integrating a transgene described herein, the transgene can be integrated within the “exon-intron junction.” If the transgene comprises cargo, the cargo will be integrated immediately before the first nucleotide in the intron. In some cases, integrating a transgene within the exon-intron junction can result in removal of sequence within the exon (e.g., integration via HR and replacement of sequence within the exon with the cargo within the transgene).

The term “full-length coding sequence” refers to a sequence of nucleic acids that encodes a protein. The full-length coding sequence can encode a protein that comprises the same number of amino acids as compared to the corresponding wild type protein or functional protein. The full-length coding sequence can encode a protein with homology to the wild type protein or functional protein

The term “partial coding sequence” as used herein refers to a sequence of nucleic acids that encodes a partial protein. The partial coding sequence can encode a protein that comprises one or less amino acids as compared to the corresponding wild type protein or functional protein. The partial coding sequence can encode a partial protein with homology to the wild type protein or functional protein. When referring to a “partial coding sequence” that is operably linked to a promoter, the term “partial coding sequence” refers to a sequence of nucleotides that encodes the N-terminus of a protein-of-interest. For example, a partial coding sequence of the ATXN2 gene, which comprises 25 exons, can include nucleotides encoding the peptide produced by exons 1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, or 1-24. When referring to a “partial coding sequence” that is operably linked to a terminator, the term “partial coding sequence” refers to a sequence of nucleotides that encodes the C-terminus of a protein-of-interest. For example, a partial coding sequence of the ATXN2 gene, can include nucleotides encoding the peptide produced by exons 2-25, 3-25, 4-25, 5-25, 6-25, 7-25, 8-25, 9-25, 10-25, 11-25, 12-25, 13-25, 14-25, 15-25, 16-25, 17-25, 18-25, 19-25, 20-25, 21-25, 22-25, 23-25, 24-25 or 25.

The term “silencing-resistant coding sequence” or “silencing-resistant partial coding sequence” refers to a sequence of nucleic acids that, when RNA is produced using the sequence as a template, the RNA is unable or less likely to be silenced by a corresponding RNAi or antisense oligonucleotide molecule. This can be due to mutations within the RNAi target site, or absence of the site.

The term “silencing agent” refers to a nucleic acid that reduces the levels of RNA or protein from an endogenous gene. The silencing agent can be in the format of RNA or DNA or a combination of RNA and DNA. The silencing agent may be shRNA, siRNA, miRNA or an antisense oligonucleotide. The silencing agent can be delivered to cells as pure RNA or DNA, or the silencing agent can be delivered on a vector which produces the silencing agent.

The terms “silencing” and “silence” and “reduced expression” and “inhibit the expression of”, in as far as they refer to silencing agent described herein refers to the at least partial suppression of the expression of an endogenous gene, as manifested by a reduction of the amount of mRNA or protein produced from the endogenous gene, as compared the amount of mRNA or protein produced by the endogenous gene in cells that have not been treated with the silencing agent. In some embodiments, the silencing agent can be a miRNA. miRNAs are a group of small non-coding RNA molecules produced endogenously miRNAs are encoded in the genome and are transcribed by RNA polymerase II (pol II) as long precursor transcripts, which are known as primary miRNAs (pri-miRNAs) of several kilobases in length. A short hairpin RNA is an artificial RNA molecule with a hairpin turn that can be used to silence target gene expression. Expression of shRNA in cells can be accomplished by delivery of plasmids, RNA, or through viral or bacterial vectors. The shRNA can be processed into siRNAs which facilitate silencing of the target mRNA transcript.

shRNA can be considered a precursor to siRNA. Whereas siRNA can be delivered directly to cells, it can also be produced by delivering shRNA, which is then processed into siRNA. Antisense oligonucleotides (ASOs) are short, synthetic, single-stranded oligodeoxynucleotides that can alter RNA and reduce expression. Therapeutic ASOs are synthetic single-stranded deoxyribonucleotide analogs, usually 15-30 bp in length. Their sequence (3′ to 5′) is antisense and complementary to the sense sequence of the target nucleotide sequence.

In some embodiments, the cells described herein comprising a gene editing event can be delivered an RNA interference agent targeting mRNA produced by an endogenous gene, wherein the endogenous gene produces a protein product that corresponds to the protein product produced by the transgene within the gene editing agents described herein. RNA interference (RNAi) refers to the process of sequence-specific post transcriptional gene silencing mediated by small interfering RNAs (siRNA), which can be produced from a precursor shRNA. Long double stranded RNA (dsRNA) in cells stimulates the activity of a ribonuclease III enzyme referred to as dicer. Dicer is involved in the processing of the long dsRNA into short pieces of siRNA. siRNAs derived from dicer activity are typically about 21-23 nucleotides in length and include duplexes of about 19 base pairs. The RNAi response also features an endonuclease complex containing a siRNA, commonly referred to as an RNA-induced silencing complex (RISC), which mediates cleavage of single stranded RNA having sequence complementary to the antisense strand of the siRNA duplex. Cleavage of the target RNA takes place in the middle of the region complementary to the antisense strand of the siRNA duplex. siRNA mediated RNAi has been studied in a variety of systems. RNAi technology has been used in mammalian cell culture, where a siRNA-mediated reduction in gene expression has been accomplished by transfecting cells with synthetic RNA oligonucleotides. The ability to use siRNA-mediated gene silencing in mammalian cells combined with the high degree of sequence specificity allows RNAi technology to be used to selectively silence expression of mutant alleles or toxic gene products in dominantly inherited diseases, including neurodegenerative diseases. Several neurodegenerative diseases, such as Parkinson's disease, Alzheimer's disease, Huntington's disease, Spinocerebellar Ataxia Type 1, Type 2, and Type 3, and dentatorubral pallidoluysian atrophy (DRLPA), have proteins identified that are involved in the overall pathogenic progression of the disease. siRNA-mediated gene silencing of mutant forms of human ataxin-3, Tau and TorsirL4, genes which cause neurodegenerative diseases such as spinocerebellar ataxia type 3, frontotemporal dementia and DYTI dystonia respectively, has been demonstrated in cultured cells.

In an embodiment, the silencing agent can be an siRNA. siRNAs may be constructed in vitro using synthetic oligonucleotides or appropriate transcription enzymes or in vivo using appropriate transcription enzymes or expression vectors. The siRNAs include a sense RNA strand and a complementary antisense RNA strand annealed together by standard Watson- Crick base-pairing interactions to form the base pairs. The sense and antisense strands of the present siRNA may be complementary single stranded RNA molecules to form a double stranded (ds) siRNA or a DNA polynucleotide encoding two complementary portions that may include a hairpin structure linking the complementary base pairs to form the siRNA. Preferably, the duplex regions of the siRNA formed by the ds RNA or by the DNA polypeptide include about 15-30 base pairs, more preferably, about 19-25 base pairs. The siRNA duplex region length may be any positive integer between 15 and 30 nucleotides. The siRNA of the invention derived from ds RNA may include partially purified RNA, substantially pure RNA, synthetic RNA, or recombinantly produced RNA, as well as altered RNA that differs from naturally- occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA of the invention may include a 3′ overhang. As used herein, a “3′ overhang” refers to at least one unpaired nucleotide extending from the 3 ‘-end of an RNA strand. In an embodiment, the siRNA may include at least one 3’ overhang of from 1 to about 6 nucleotides (which includes ribonucleotides or deoxynucleotides) in length, preferably from 1 to about 5 nucleotides in length, more preferably from 1 to about 4 nucleotides in length, and particularly preferably from about 2 to about 4 nucleotides in length. Both strands of the siRNA molecule may include a 3′ overhang, the length of the overhangs can be the same or different for each strand. The 3′ overhang may be present on both strands of the siRNA, and is 2 nucleotides in length. The 3′ overhangs may also be stabilized against degradation. For example, the overhangs may be stabilized by including purine nucleotides, such as adenosine or guanosine nucleotides, by substitution of pyrimidine nucleotides by modified analogues, e.g., substitution of uridine nucleotides in the 3′ overhangs with 2′-deoxythymidine, is tolerated and does not affect the efficiency of RNAi degradation, hi particular, the absence of a 2′ hydroxyl in the 2′-deoxythymidine significantly enhances the nuclease resistance of the 3′ overhang in tissue culture medium.

In some embodiments, the RNA duplex portion of the siRNA may be part of a hairpin structure. The hairpin structure may further contain a loop portion positioned between the two sequences that form the duplex. The loop can vary in length. In some embodiments, the loop may be 5, 6, 7, 8, 9, 10, 11, 12 or 13 nucleotides in length. The hairpin structure may also contain 3′ or 5′ overhang portions. In some embodiments, the overhang is a 3′ or a 5′ overhang 0, 1, 2, 3, 4 or 5 nucleotides in length.

In some embodiments, the siRNA of the present invention may also be expressed from a recombinant plasmid either as two separate, complementary RNA molecules, or as a single RNA molecule with two complementary regions. Selection of vectors suitable for expressing siRNA of the invention, methods for inserting nucleic acid sequences for expressing the siRNA into the plasmid, and methods of delivering the recombinant plasmid to the cells of interest are within the skill in the art. The siRNA of the present invention may be a polynucleotide sequence cloned into a plasmid vector and expressed using any suitable promoter. Suitable promoters for expressing siRNA of the invention from a plasmid include, but are not limited to, the H1 and U6 RNA pol III promoter sequences and viral promoters including the viral LTR, adenovirus, SV40, and CMV promoters. Additional promoters known to one of skill in the art may also be used, including tissue specific, inducible or regulatable promoters for expression of the siRNA in a particular tissue or in a particular intracellular environment. The vector may also include additional regulatory or structural elements, including, but not limited to introns, enhancers, and polyadenylation sequences. These elements may be included in the DNA as desired to obtain optimal performance of the siRNA in the cell and may or may not be necessary for the function of the DNA. Optionally, a selectable marker gene or a reporter gene may be included either with the siRNA encoding polynucleotide or as a separate plasmid for delivery to the target cells. Additional elements known to one of skill in the art may also be included.

The siRNA may also be expressed from a polynucleotide sequence cloned into a viral vector that may include the elements described above. Suitable viral vectors for gene delivery to a cell include, but are not limited to, replication-deficient viruses that are capable of directing synthesis of all virion proteins, but are incapable of making infections particles. Exemplary viruses include, but are not limited to lentiviruses, adenoviruses, adeno-associated viruses, retroviruses, and alphaviruses.

The siRNA may also be delivered to cells in vitro or in vivo using lipid nanoparticles. When using lipid nanoparticles, the siRNA may be in the form of purified RNA. Physical methods to introduce a preselected DNA or RNA duplex into a host cell further include, but are not limited to, calcium phosphate precipitation, lipofection, DEAE-dextran, particle bombardment, microinjection, electroporation, immunoliposomes, lipids, cationic lipids, phospholipids, or liposomes and the like. One skilled in the art will understand that any method may be used to deliver the DNA or RNA duplex into the cell. One mode of administration to the CNS uses a convection-enhanced delivery (CED) system. This method includes: a) creating a pressure gradient during interstitial infusion into white matter to generate increased flow through the brain interstitium (convection-supplementing simple diffusion); b) maintaining the pressure gradient over a lengthy period of time (24 hours to 48 hours) to allow radial penetration of the migrating compounds (such as: neurotrophic factors, antibodies, growth factors, genetic vectors, enzymes, etc.) into the gray matter; and c) increasing drug concentrations by orders of magnitude over systemic levels. Using a CED system, DNA , RNA duplexes or viruses can be delivered to many cells over large areas of the brain. Any CED device may be appropriate for delivery of DNA, RNA or viruses. In some embodiments, the device is an osmotic pump or an infusion pump. Both osmotic and infusion pumps are commercially available from a variety of suppliers, for example Alzet Corporation, Hamilton Corporation, Alza, Inc., Palo Alto, Calif). Biological methods to introduce the nucleotide of interest into a host cell include the use of DNA and RNA viral vectors. For mammalian gene therapy, it is desirable to use an efficient means of inserting a copy gene into the host genome. Viral vectors have become the most widely used method for inserting genes into mammalian, e.g., human cells. Delivery of the recombinant nucleotides to the host cell may be confirmed by a variety of assays known to one of skill in the art. Assays include Southern and Northern blotting, RT-PCR, PCR, ELISA, and Western blotting, by way of example.

In some embodiments, different methods can be used to introduce shRNA into cells, including viral and non-viral delivery methods. shRNA can be delivered in vivo using plasmid or viral vectors. The plasmid or viral vectors can include sequence that results in expression of shRNA within a cell, which is followed by processing of the shRNA into siRNA. In some embodiments, the shRNA can be delivered on lentiviral vectors, adenoviral vectors, retroviral vectors, or adeno-associated viral vectors.The methods and compositions described in this document can use transgenes having a cargo sequence. The term “cargo” can refer to elements such as the complete or partial coding sequence of a gene, a partial sequence of a gene comprising single-nucleotide polymorphisms relative to the WT or altered target, a splice acceptor, a splice donor, a promoter, a terminator, a transcriptional regulatory element, an RNAi cassette, purification tags (e.g., glutathione-S-transferase, poly(His), maltose binding protein, Strep-tag, Myc-tag, AviTag, HA-tag, or chitin binding protein) or reporter genes (e.g., GFP, RFP, lacZ, cat, luciferase, puro, neomycin). As defined herein, “cargo” can refer to the sequence within a transgene that is integrated at a target site. For example, “cargo” can refer to the sequence on a transgene between two homology arms, between two rare-cutting endonuclease target sites or between two ITR sequences.

The term “homology arm” or “homology arms” refers to a sequence of nucleic acids that comprises homology to a second nucleic acid. Homology arms, for example, can be present on a donor molecule. Homology arms can facilitate homologous recombination with the second nucleic acid. In an embodiment, homology arms can have homology to an endogenous gene.

The term “bidirectional terminator” refers to a terminator that can terminate RNA polymerase transcription in either the sense or antisense direction. In contrast to two unidirectional terminators in tail-to-tail orientation, a bidirectional terminator can comprise a non-chimeric sequence of DNA. Examples of bidirectional terminators include the ARO4, TRP1, TRP4, ADH1, CYC1, GAL1, GAL7, and GAL10 terminator.

The term “bidirectional promoter” refers to a promoter that can initiate RNA polymerase transcription in either the sense or antisense direction. In contrast to two unidirectional promoters in head-to-head orientation, a bidirectional promoter can comprise a non-chimeric sequence of DNA. Examples of bidirectional promoters include those described in Trinklein et al., Genome Res. 14:62-66, 2004, the entire disclosure of which, except for any definitions, disclaimers, disavowals, and inconsistencies, is incorporated herein by reference.

A 5′ or 3′ end of a nucleic acid molecule references the directionality and chemical orientation of the nucleic acid. As defined herein, the “5′ end of a gene” can comprise the exon with the start codon, but not the exon with the stop codon. As defined herein, the “3′ end of a gene” can comprise the exon with the stop codon, but not the exon with the start codon. The term “RNAi” refers to RNA interference, a process that uses RNA molecules to inhibit or reduce gene expression or translation. RNAi can be induced with the use of small interfering RNAs (siRNA) or short hairpin RNAs (shRNA).

The term “ATXN3” gene refers to a gene that encodes the enzyme ataxin-3. A representative sequence of the ATXN3 gene can be found with NCBI Reference Sequence: NG_008198.2. Specifically, exon 1 includes the sequence from 1 to 54. Exon 2 includes the sequence from 9745 to 9909. Exon 3 includes the sequence from 10446 to 10490. Exon 4 includes the sequence from 12752 to 12837. Exon 5 includes the sequence from 13265 to 13331. Exon 6 includes the sequence from 17766 to 17853. Exon 7 includes the sequence from 23325 to 23457. Exon 8 includes the sequence from 24117 to 24283. Exon 9 includes the sequence from 25522 to 25618. Exon 10 includes the sequence from 35530 to 35648. Exon 11 includes the sequence from 42169 to 48031. Intron 1 includes the sequence from 55 to 9744. Intron 2 includes the sequence from 9910 to 10445. Intron 3 includes the sequence from 10491 to 12751. Intron 4 includes the sequence from 12838 to 13264. Intron 5 includes the sequence from 13332 to 17765. Intron 6 includes the sequence from 17854 to 23324. Intron 7 includes the sequence from 23458 to 24116. Intron 8 includes the sequence from 24284 to 25521. Intron 9 includes the sequence from 25619 to 35529. Intron 10 includes the sequence from 35649 to 42168.

The term “CACNA1A” gene refers to a gene that encodes the calcium voltage-gated channel subunit alphal A protein. A representative sequence of the

CACNA1A gene can be found with NCBI Reference Sequence: NG_011569.1. Specifically, exon 1 includes the sequence from 1 to 529. Exon 2 includes the sequence from 51249 to 51354. Exon 3 includes the sequence from 53446 to 53585. Exon 4 includes the sequence from 134682 to 134773. Exon 5 includes the sequence from 140992 to 141144. Exon 6 includes the sequence from 146662 to 146855. Exon 7 includes the sequence from 170552 to 170655. Exon 8 includes the sequence from 171968 to 172083. Exon 9 includes the sequence from 173536 to 173592. Exon 10 includes the sequence from 176125 to 176217. Exon 11 includes the sequence from 189140 to 189349. Exon 12 includes the sequence from 193680 to 193792. Exon 13 includes the sequence from 197933 to 198045. Exon 14 includes the sequence from 198210 to 198341. Exon 15 includes the sequence from 198607 to 198679. Exon 16 includes the sequence from 202577 to 202694. Exon 17 includes the sequence from 202848 to 202915. Exon 18 includes the sequence from 205805 to 205911. Exon 19 includes the sequence from 207108 to 207917. Exon 20 includes the sequence from 219495 to 219958. Exon 21 includes the sequence from 221255 to 221393. Exon 22 includes the sequence from 223065 to 223194. Exon 23 includes the sequence from 229333 to 229392. Exon 24 includes the sequence from 230505 to 230611. Exon 25 includes the sequence from 243628 to 243727. Exon 26 includes the sequence from 244851 to 245011. Exon 27 includes the sequence from 246760 to 246897. Exon 28 includes the sequence from 248910 to 249111. Exon 29 includes the sequence from 251202 to 251366. Exon 30 includes the sequence from 253360 to 253470. Exon 31 includes the sequence from 261196 to 261279. Exon 32 includes the sequence from 270731 to 270847. Exon 33 includes the sequence from 271187 to 271252. Exon 34 includes the sequence from 271425 to 271540. Exon 35 includes the sequence from 274601 to 274751. Exon 36 includes the sequence from 276252 to 276379. Exon 37 includes the sequence from 277666 to 277762. Exon 38 includes the sequence from 281689 to 281794. Exon 39 includes the sequence from 291853 to 291960. Exon 40 includes the sequence from 292128 to 292228. Exon 41 includes the sequence from 293721 to 293830. Exon 42 includes the sequence from 293939 to 294077. Exon 43 includes the sequence from 294245 to 294358. Exon 44 includes the sequence from 295809 to 295844. Exon 45 includes the sequence from 296963 to 297149. Exon 46 includes the sequence from 297452 to 297705. Exon 47 includes the sequence from 298413 to 300019. Intron 1 includes the sequence from 530 to 51248. Intron 2 includes the sequence from 51355 to 53445. Intron 3 includes the sequence from 53586 to 134681. Intron 4 includes the sequence from 134774 to 140991. Intron 5 includes the sequence from 141145 to 146661. Intron 6 includes the sequence from 146856 to 170551. Intron 7 includes the sequence from 170656 to 171967. Intron 8 includes the sequence from 172084 to 173535. Intron 9 includes the sequence from 173593 to 176124. Intron 10 includes the sequence from 176218 to 189139. Intron 11 includes the sequence from 189350 to 193679. Intron 12 includes the sequence from 193793 to 197932. Intron 13 includes the sequence from 198046 to 198209. Intron 14 includes the sequence from 198342 to 198606. Intron 15 includes the sequence from 198680 to 202576. Intron 16 includes the sequence from 202695 to 202847. Intron 17 includes the sequence from 202916 to 205804. Intron 18 includes the sequence from 205912 to 207107. Intron 19 includes the sequence from 207918 to 219494. Intron 20 includes the sequence from 219959 to 221254. Intron 21 includes the sequence from 221394 to 223064. Intron 22 includes the sequence from 223195 to 229332. Intron 23 includes the sequence from 229393 to 230504. Intron 24 includes the sequence from 230612 to 243627. Intron 25 includes the sequence from 243728 to 244850. Intron 26 includes the sequence from 245012 to 246759. Intron 27 includes the sequence from 246898 to 248909. Intron 28 includes the sequence from 249112 to 251201. Intron 29 includes the sequence from 251367 to 253359. Intron 30 includes the sequence from 253471 to 261195. Intron 31 includes the sequence from 261280 to 270730. Intron 32 includes the sequence from 270848 to 271186. Intron 33 includes the sequence from 271253 to 271424. Intron 34 includes the sequence from 271541 to 274600. Intron 35 includes the sequence from 274752 to 276251. Intron 36 includes the sequence from 276380 to 277665. Intron 37 includes the sequence from 277763 to 281688. Intron 38 includes the sequence from 281795 to 291852. Intron 39 includes the sequence from 291961 to 292127. Intron 40 includes the sequence from 292229 to 293720. Intron 41 includes the sequence from 293831 to 293938. Intron 42 includes the sequence from 294078 to 294244. Intron 43 includes the sequence from 294359 to 295808. Intron 44 includes the sequence from 295845 to 296962. Intron 45 includes the sequence from 297150 to 297451. Intron 46 includes the sequence from 297706 to 298412.

The term “ATXN2” gene refers to a gene that encodes the enzyme ataxin-2. A representative sequence of the ATXN2 gene can be found with NCBI Reference Sequence: NG_011572.3.Specifically, exon 1 includes the sequence from 282 to 532. Exon 2 includes the sequence from 43397 to 43433. Exon 3 includes the sequence from 45099 to 45158. Exon 4 includes the sequence from 46339 to 46410. Exon 5 includes the sequence from 46886 to 47036. Exon 6 includes the sequence from 74000 to 74124. Exon 7 includes the sequence from 78343 to 78434. Exon 8 includes the sequence from 79240 to 79437. Exon 9 includes the sequence from 80889 to 81067. Exon 10 includes the sequence from 82953 to 83162. Exon 11 includes the sequence from 85777 to 85959. Exon 12 includes the sequence from 88734 to 88931. Exon 13 includes the sequence from 89318 to 89425. Exon 14 includes the sequence from 89697 to 89767. Exon 15 includes the sequence from 110536 to 110840. Exon 16 includes the sequence from 112492 to 112555. Exon 17 includes the sequence from 113451 to 113603. Exon 18 includes the sequence from 113985 to 114051. Exon 19 includes the sequence from 128574 to 128758. Exon 20 includes the sequence from 129076 to 129208. Exon 21 includes the sequence from 134601 to 134654. Exon 22 includes the sequence from 141957 to 142102. Exon 23 includes the sequence from 143060 to 143287. Exon 24 includes the sequence from 145471 to 145639. Exon 25 includes the sequence from 146476 to 146504. Intron 1 includes the sequence from 533 to 43396. Intron 2 includes the sequence from 43434 to 45098. Intron 3 includes the sequence from 45159 to 46338. Intron 4 includes the sequence from 46411 to 46885. Intron 5 includes the sequence from 47037 to 73999. Intron 6 includes the sequence from 74125 to 78342. Intron 7 includes the sequence from 78435 to 79239. Intron 8 includes the sequence from 79438 to 80888. Intron 9 includes the sequence from 81068 to 82952. Intron 10 includes the sequence from 83163 to 85776. Intron 11 includes the sequence from 85960 to 88733. Intron 12 includes the sequence from 88932 to 89317. Intron 13 includes the sequence from 89426 to 89696. Intron 14 includes the sequence from 89768 to 110535. Intron 15 includes the sequence from 110841 to 112491. Intron 16 includes the sequence from 112556 to 113450. Intron 17 includes the sequence from 113604 to 113984. Intron 18 includes the sequence from 114052 to 128573. Intron 19 includes the sequence from 128759 to 129075. Intron 20 includes the sequence from 129209 to 134600. Intron 21 includes the sequence from 134655 to 141956. Intron 22 includes the sequence from 142103 to 143059. Intron 23 includes the sequence from 143288 to 145470. Intron 24 includes the sequence from 145640 to 146475. Examples of pathogenic mutations in ATXN2 include a CAG trinucleotide expansion in exon 1 (32 or more CAG repeats). Examples of non-pathogenic mutations include ClinVar accession number VCV000522367, VCV000522368, VCV000522369, VCV000522370, VCV000128509, VCV000128508, VCV000128507, VCV000218618.

The term “SNCA” gene refers to a gene that encodes the protein synuclein alpha. A representative sequence of the SNCA gene can be found with NCBI Reference Sequence: NG_011851.1. Specifically, exon 1 includes the sequence from 1 to 200. Exon 2 includes the sequence from 1470 to 1615. Exon 3 includes the sequence from 8978 to 9019. Exon 4 includes the sequence from 14774 to 14916. Exon 5 includes the sequence from 107885 to 107968. Exon 6 includes the sequence from 110502 to 113063. Intron 1 includes the sequence from 201 to 1469. Intron 2 includes the sequence from 1616 to 8977. Intron 3 includes the sequence from 9020 to 14773. Intron 4 includes the sequence from 14917 to 107884. Intron 5 includes the sequence from 107969 to 110501. The start codon is present in intron 2. Examples of pathogenic mutations in SNCA include a duplication or triplication of the gene, A53T, G51D, E46K, and A30P. Examples of non-pathogenic mutations include ClinVar accession number VCV000350063, VCV000350064, VCV000350086, and VCV000350093.

As defined herein, a SOD1 gene refers to a gene that produces the enzyme superoxide dismutase. A representative sequence of the SOD1 gene can be found with NCBI Reference Sequence: NG_008689.1. Specifically, exon 1 includes the sequence from 5001 to 5220. Exon 2 includes sequence from 9169 to 9265. Exon 3 includes sequence from 11828 to 11897. Exon 4 includes sequence from 12637 to 12754. Exon 5 includes sequence from 13850 to 14310. Intron 1 includes sequence from 5221 to 9168. Intron 2 includes sequence from 9170 to 11827. Intron 3 includes sequence from 11898 to 12636. Intron 4 includes sequence from 12755 to 12849. The methods described herein provide transgenes for integrating into the SOD1 gene. The transgenes can comprise a promoter, partial SOD1 coding sequence and splice donor, and the integration site can be within intron 1, 2, 3 or 4 of the endogenous SOD1 gene. Further the transgenes can comprise an RNAi cassette targeting the endogenous SOD1 transcripts, a promoter, a partial SOD1 coding sequence (resistant to silencing by the RNAi cassette, and a splice donor. The transgene can be integrated within intron 1, 2, 3 or 4 of the endogenous SOD1 gene. Also, the transgenes can comprise a splice acceptor, partial SOD1 coding sequence (resistant to silencing by an RNAi cassette), a terminator, and an RNAi cassette targeting the endogenous SOD1 transcripts. The transgene can be integrated within intron 1, 2, 3, or 4 of the endogenous SOD1 gene. Examples of pathogenic mutations in SOD1 include ASV, C7F, G13R, G17S, E22K, G38R, L39V, G42S, F46C, H47R, G73S, H81R, L85V, G86R, G94R, E101G, I105F, and L107V. Examples of non-pathogenic mutations include ClinVar accession number VCV000440292, VCV000256202, VCV000586633, and VCV000395173.

As defined herein, a RHO gene refers to a gene that produces the protein rhodopsin. A representative sequence of the RHO gene can be found with NCBI Reference Sequence: NC_000003.12. Specifically, exon 1 includes the sequence from 1 to 456. Exon 2 includes the sequence from 2238 to 2406. Exon 3 includes the sequence from 3613 to 3778. Exon 4 includes the sequence from 3895 to 4134. Exon 5 includes the sequence from 4970 to 6706. Intron 1 includes the sequence from 457 to 2237. Intron 2 includes the sequence from 2407 to 3612. Intron 3 includes the sequence from 3779 to 3894. Intron 4 includes the sequence from 4135 to 4969. The methods described herein provide transgenes for integrating into the RHO gene. The transgenes can comprise a promoter, partial RHO coding sequence and splice donor, and the integration site can be within intron 1, 2, 3 or 4 of the endogenous RHO gene. Further the transgenes can comprise an RNAi cassette targeting the endogenous RHO transcripts, a promoter, a partial RHO coding sequence (resistant to silencing by the RNAi cassette, and a splice donor. The transgene can be integrated within intron 1, 2, 3 or 4 of the endogenous RHO gene. Also, the transgenes can comprise a splice acceptor, partial RHO coding sequence (resistant to silencing by an RNAi cassette), a terminator, and an RNAi cassette targeting the endogenous RHO transcripts. The transgene can be integrated within intron 1, 2, 3, or 4 of the endogenous RHO gene. Examples of pathogenic mutations in RHO include ClinVar accession number VCV000013039, VCV000013031, VCV000013017, VCV000013042, VCV000013018, VCV000625297, VCV000013055, VCV000013013, VCV000013019, VCV000013047, VCV000013016, VCV000013020, VCV000013021, VCV000013045, VCV000013054, VCV000625301, VCV000013038, VCV000013022, VCV000013035, VCV000013048, VCV000373094, VCV000013028, VCV000279882, VCV000013024, VCV000013046, VCV000029875, VCV000013049, VCV000417867, VCV000013050, VCV000143080, VCV000625303, VCV000013025, VCV000196282, VCV000013033, VCV000590911, VCV000143081, VCV000013023, VCV000013026, VCV000013043, VCV000013027, VCV000013051, VCV000013034, VCV000013036, VCV000636084, VCV000013030, VCV000523376, VCV000013044, VCV000013029, VCV000419250, VCV000013056, VCV000013052, VCV000013015, VCV000013053, VCV000013032, VCV000013014, VCV000605502, VCV000605497, VCV000442401, VCV000442400, VCV000154258, and VCV000145614. Examples of non-pathogenic mutations include ClinVar accession number VCV000343272, VCV000256383, VCV000281512, VCV000256384, VCV000256382, VCV000343286, VCV000343290, VCV000343302, VCV000343303, VCV000343306, and VCV000606153.

As defined herein, a C9orf72 gene refers to a gene that produces a protein in various tissues and has been associated with amyotrophic lateral sclerosis. A representative sequence of the C9orf72 gene can be found with NCBI Reference Sequence: NG_031977.1. Specifically, exon 1 includes the sequence from 1 to 158. Exon 2 includes the sequence from 6703 to 7190. Exon 3 includes the sequence from 8277 to 8336. Exon 4 includes the sequence from 11391 to 11486. Exon 5 includes the sequence from 12218 to 12282. Exon 6 includes the sequence from 13568 to 13640. Exon 7 includes the sequence from 15260 to 15376. Exon 8 includes the sequence from 17071 to 17306. Exon 9 includes the sequence from 23160 to 23217. Exon 10 includes the sequence from 25201 to 25310. Exon 11 includes the sequence from 25445 to 27321. Intron 1 includes the sequence from 159 to 6702. Intron 2 includes the sequence from 7191 to 8276. Intron 3 includes the sequence from 8337 to 11390. Intron 4 includes the sequence from 11487 to 12217. Intron 5 includes the sequence from 12283 to 13567. Intron 6 includes the sequence from 13641 to 15259. Intron 7 includes the sequence from 15377 to 17070. Intron 8 includes the sequence from 17307 to 23159. Intron 9 includes the sequence from 23218 to 25200. Intron 10 includes the sequence from 25311 to 25444. The methods described herein provide transgenes for integrating into the C9orf72 gene. The transgenes can comprise a promoter, partial C9orf72 coding sequence and splice donor, and the integration site can be within intron 1, 2, 3, 4, 5, 6, 7, 8, 9, or 10 of the endogenous C9orf72 gene. Further the transgenes can comprise an RNAi cassette targeting the endogenous C9orf72 transcripts, a promoter, a partial C9orf72 coding sequence (resistant to silencing by the RNAi cassette, and a splice donor. The transgene can be integrated within intron 1, 2, 3, 4, 5, 6, 7, 8, 9, or 10 of the endogenous C9orf72 gene. Also, the transgenes can comprise a splice acceptor, partial C9orf72 coding sequence (resistant to silencing by an RNAi cassette), a terminator, and an RNAi cassette targeting the endogenous C9orf72 transcripts. The transgene can be integrated within intron 1, 2, 3, 4, 5, 6, 7, 8, 9, or 10 of the endogenous C9orf72 gene. Examples of pathogenic mutations in C9orf72 include the duplication, triplication or quadruplication of the C9or72 gene, or expansion of the GGGGCC repeat. Examples of non-pathogenic mutations include ClinVar accession number VCV000366486, VCV000366521, VCV000366524, VCV000183033, and VCV000611705.

As defined herein, a CHRNA1 gene refers to a gene that produces the protein cholinergic receptor nicotinic alpha 1 subunit. A representative sequence of the CHRNA1 gene can be found with NCBI Reference Sequence: NG_008172.1. As defined herein, a CHRND gene refers to a gene that produces the protein cholinergic receptor nicotinic delta subunit. A representative sequence of the CHRND gene can be found with NCBI Reference Sequence: NG_008028.1. As defined herein, a CHRNE gene refers to a gene that produces the protein cholinergic receptor nicotinic epsilon subunit. A representative sequence of the CHRNE gene can be found with NCBI Reference Sequence: NG_008029.2. As defined herein, a CHRNB1 gene refers to a gene that produces the protein cholinergic receptor nicotinic beta 1 subunit. A representative sequence of the CHRNB1 gene can be found with NCBI Reference Sequence: NG_008026.1. As defined herein, a PRPS1 gene refers to a gene that produces the protein phosphoribosyl pyrophosphate synthetase 1. A representative sequence of the PRPS1 gene can be found with NCBI Reference Sequence: NG_008407.1. As defined herein, a LRRK2 gene refers to a gene that produces the protein leucine rich repeat kinase 2. A representative sequence of the LRRK2 gene can be found with NCBI Reference Sequence: NG_011709.1. As defined herein, a STIM1 gene refers to a gene that produces the protein stromal interaction molecule 1. A representative sequence of the STIM1 gene can be found with NCBI Reference Sequence: NG_016277.1. As defined herein, a FGFR3 gene refers to a gene that produces the protein fibroblast growth factor receptor 3. A representative sequence of the FGFR3 gene can be found with NCBI Reference Sequence: NG_012632.1. As defined herein, a MECP2 gene refers to a gene that produces the protein methyl-CpG binding protein 2. A representative sequence of the MECP2 gene can be found with NCBI Reference Sequence: NG_007107.2. As defined herein, an ATXN1 gene refers to a gene that produces the protein ataxin 1. A representative sequence of the ATXN1 gene can be found with NCBI Reference Sequence: NG_011571.1. As defined herein, an ATXN3 gene refers to a gene that produces the protein ataxin 3. A representative sequence of the ATXN3 gene can be found with NCBI Reference Sequence: NG_008198.2. As defined herein, a CACNA1A gene refers to a gene that produces the protein calcium voltage-gated channel subunit alphal A. A representative sequence of the CACNA1A gene can be found with NCBI Reference Sequence: NG_011569.1. As defined herein, an ATXN7 gene refers to a gene that produces the protein ataxin 7. A representative sequence of the ATXN7 gene can be found with NCBI Reference Sequence: NG_008227.1. As defined herein, a TBP gene refers to a gene that produces the protein TATA-box binding protein. A representative sequence of the TBP gene can be found with NCBI Reference Sequence: NG_008165.1. As defined herein, an HTT gene refers to a gene that produces the protein huntingtin. A representative sequence of the HTT gene can be found with NCBI Reference Sequence: NG_009378.1. As defined herein, an AR gene refers to a gene that produces the protein androgen receptor. A representative sequence of the AR gene can be found with NCBI Reference Sequence: NG_009014.2. As defined herein, an FXN gene refers to a gene that produces the protein frataxin. A representative sequence of the FXN gene can be found with NCBI Reference Sequence: NG_008845.2. As defined herein, a DMPK gene refers to a gene that produces the protein DM1 protein kinase. A representative sequence of the DMPK gene can be found with NCBI Reference Sequence: NG_009784.1. As defined herein, a PABPN1 gene refers to a gene that produces the protein poly(A) binding protein nuclear 1. A representative sequence of the PABPN1 gene can be found with NCBI Reference Sequence: NG_008239.1. As defined herein, an ATXN8 gene refers to a gene that produces the protein ataxin 8. A representative sequence of the ATXN8 gene can be found at the genomic coordinates (GRCh38): 13:54,700,000-72,800,000.

As described herein, the term “silencing-resistant partial coding sequence” refers to a partial coding sequence with mutations compared to the homologous sequence from the corresponding endogenous gene, wherein the mutations are designed to prevent or reduce silencing by a corresponding RNAi cassette. The mutations can be the insertion, substitution, or deletion of nucleotides within the DNA sequence which encodes the target RNA sequence. The mutations can be sufficient to prevent or reduce hybridization of a short RNA molecule to the RNA transcript.

As defined herein, “lack of the sequence” when referring to a silencing-resistant partial coding sequence refers to the deletion of one or more nucleotides within the corresponding RNAi target site. For example, if the RNAi targets the transcript produced by the sequence GGTATCAAGACTACGAAC (within the exon of an endogenous gene), then this sequence can also be present within the partial coding sequence of the transgenes described herein. To prevent silencing of modified genes, the RNAi target sequence within the partial coding sequence within the transgene can be modified. Specifically, the site can be mutated by insertion, substitution or deletion of nucleotides within the site. If the mutation is a deletion, then one or more of the nucleotides can be deleted. In instances where the nucleotides are deleted, it is preferred that the deletion is designed to be an in-frame deletion which doesn't eliminate protein function.

As defined herein, “administering” can refer to the delivery, the providing, or the introduction of exogenous molecules into a cell. If a transgene or a rare-cutting endonuclease is administered to a cell, then the transgene or rare-cutting endonuclease is delivered to, provided to, or introduced into the cell. The rare-cutting endonuclease can be administered as purified protein, nucleic acid, or a mixture of purified protein and nucleic acid. The nucleic acid (i.e., RNA or DNA), can encode for the rare-cutting endonuclease, or a part of a rare-cutting endonuclease (e.g., a gRNA). The administering can be achieved though methods such as lipid-mediated transfer, electroporation, direct injection, cell fusion, particle bombardment, calcium phosphate co-precipitation, DEAE-dextran-mediated transfer, viral vector-mediated transfer, or any means suitable of delivering purified protein or nucleic acids, or a mixture of purified protein and nucleic acids, to a cell. Administer can refer to the delivery, the providing, or the introduction of exogenous molecules to an organism, which will then result in administering of exogenous molecules to cells within the organism.

Reduced expression is expressed as expression that is less than the expression that occurs in otherwise comparable untreated cells. For example, in certain instances, expression of the endogenous gene is reduced by at least about 20%, 25%, 35%, or 50% by administration of the silencing agents described herein, as compared to the expression of the endogenous gene in a cell not administered the silencing agents. In some embodiments, expression of the endogenous gene is reduced by at least about 60%, 70%, or 80% by administration of the silencing agent, as compared to the expression of the endogenous gene in a cell not administered the silencing agents. In some embodiments, expression of the endogenous gene is reduced by at least about 85%, 90%, or 95% by administration of the silencing agent, as compared to the expression of the endogenous gene in a cell not administered the silencing agents. In some embodiments mRNA encoded by the endogenous gene is reduced. . In some embodiments protein encoded by the endogenous gene is reduced. Detecting reduced levels of mRNA or protein can be assayed with methods common in the art, including Northern blotting, Western blotting, reverse transcription polymerase chain reaction (RT-PCR), RNA seq, or DNA microarray.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seql.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C: \B12seq c:\seql.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seql.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\1312seq c:\seql.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. The percent sequence identity value is rounded to the nearest tenth.

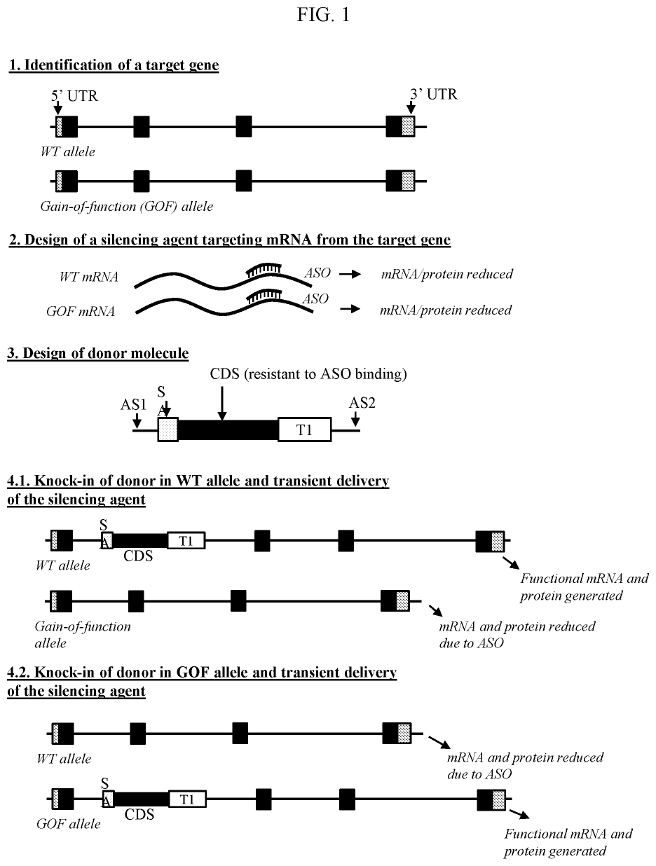

In one aspect, the methods described herein provide novel approaches for correcting gene expression from gain-of-function disorders. The methods combine gene editing and gene silencing to correct and silence, respectively, the mRNA and protein produced by an endogenous gene comprising a gain-of-function mutation. The methods include administering a transgene to the cell, wherein the transgene comprises a first coding sequence that encodes an amino acid sequence that is homologous to an amino acid sequence encoded by the endogenous gene or to a polypeptide fragment thereof, integrating the transgene into a first allele of an endogenous gene to create a modified first allele, and administering a silencing agent to the cell that reduces expression of the endogenous gene, wherein the first coding sequence that is not silenced by the silencing agent, wherein the modified first allele is expressed at a higher level than the second allele (FIGS. 1 - 2).

In some embodiments, this document features a transgene with coding sequence. The coding sequence can be a partial or full-length coding sequence. The coding sequence can be operably linked to a splice acceptor, splice donor, terminator, promoter, or 2A sequence. In transgenes comprising a coding sequence operably linked to a terminator, the coding sequence is not operably linked to a promoter. In transgenes comprising a coding sequence operably linked to a promoter, the coding sequence is not operably linked to a terminator. In transgenes comprising a full-length coding sequence, the coding sequence is operably linked to a terminator, but not a promoter. The partial or full-length coding sequence can encode the same amino acids as the amino acids produced by the target endogenous gene, or the partial or full-length coding sequence can encode amino acids that are 50%, 60%, 70%, 80%, 90%, 95%, 96,%, 97%, 98%, or 99% homologous to the amino acids encoded by the endogenous gene. In other embodiments, the partial or full-length coding sequence can encode different amino acids as the target endogenous gene. For example, the coding sequence can be a full-length coding sequence for an endogenous gene such as TTR, and the endogenous gene can be albumin. In another example, the coding sequence can be a partial or full-length SERPINA coding sequence and the endogenous gene can be albumin. Following or concurrent to administration of the transgene, the cells can be administered a silencing agent. The partial or full-length coding sequence can be made resistant to the silencing agent by deleting or modifying the DNA sequence corresponding to the mRNA target site. As defined herein, the mRNA target site for the silencing agent refers to a sequence of nucleic acids within an mRNA that is the target for a silencing agent to bind to or interact with. This mRNA target site can be modified such that the silencing agent can no longer bind to or interact with the mRNA. One way to modify an mRNA target site is to alter the corresponding DNA sequence that produces the mRNA with mutations (i.e., synonymous mutations, deletions or insertions). By way of example, a transgene carrying a coding sequence for SERPINA can be integrated into an endogenous albumin gene, wherein the SERPINA coding sequence is expressed from the transgene and also two alleles from endogenous genes. A silencing agent can be designed and administered to the cells to silence SERPINA expression. To prevent the silencing agent from silencing the SEPINA expressed from the transgene, the DNA coding sequence of SERPINA present on the transgene can comprise synonymous mutations. The synonymous mutations can be within the DNA sequence that corresponds to the mRNA target site. By way of another example, a transgene carrying a coding sequence for RHO can be integrated into an endogenous RHO gene, wherein the RHO coding sequence is expressed from the transgene and one of the endogenous RHO alleles (assuming integration occurs in one allele). A silencing agent can be designed and administered to the cells to silence RHO expression. To prevent the silencing agent from silencing the RHO expressed from the transgene, the DNA coding sequence of RHO present on the transgene can comprise synonymous mutations. The synonymous mutations can be within the DNA sequence that corresponds to the mRNA target site. By way of another example, one way to prevent silencing by a silencing agent is to have the silencing agent target mRNA produced by the 3′ UTR of an endogenous gene, and produce a transgene that does not comprise the 3′ UTR of the endogenous gene.

The silencing agent is designed to reduce the expression of the endogenous gene that comprises amino acids with homology to the partial or full-length coding sequence within the transgene. For example, if a transgene comprising a full-length

TTR coding sequence is integrated within the albumin gene, then the silencing agent is designed to reduce expression of the endogenous TTR gene. If a transgene comprises a partial TTR coding sequence is integrated within the endogenous TTR gene, then the silencing agent is designed to reduce expression of the endogenous TTR gene. The silencing agent can reduce the expression of each allele within an unmodified endogenous gene at similar efficiencies. The silencing agent can reduce the expression of each allele of an unmodified endogenous gene within a cell by about 10%, about 20%, about 30%, about 40%, about 50%, about 60%, about 70%, about 80%, about 90%, about 95%, about 99%, compared to the expression of each allele in a cell that is not administered a silencing agent. The silencing agent can reduce expression of each allele of an unmodified endogenous gene within a cell by about 5-10%, about 10-20%, about 20-30%, about 30-40%, about 40-50%, about 50-60%, about 60-70%, about 70-80%, about 80-90%, about 90-95%, about 95-99%, compared to the expression of each allele in a cell that is not administered a silencing agent. The silencing agent can reduce expression of each allele of an unmodified endogenous gene within a cell by greater than 10%, greater than 20%, greater than 30%, greater than 40%, greater than 50%, greater than 60%, greater than 70%, greater than 80%, greater than 90%, greater than 95%, greater than 99%, compared to the expression of each allele in a cell that is not administered a silencing agent.

In some embodiments, the transgene is integrated into one or more alleles of an endogenous gene. The transgene can be integrated into an endogenous gene using any method of site-specific integration known in the art, including targeted integration using rare-cutting endonucleases. In some embodiments, a rare-cutting endonuclease is designed to cleave an intron within both alleles of an endogenous gene. Cells administered the rare-cutting endonuclease and a transgene can have one or more of the alleles comprising an integration event of the transgene. By way of example, a cell comprising an endogenous gene having a first allele and a second allele can have different integration outcomes. In one case, the first allele can comprise the transgene, while the second allele remains WT or comprises a rare-cutting nuclease induced NHEJ mutation. In a second case, the second allele can comprise the transgene, while the first allele remains WT or comprises a rare-cutting nuclease induced NHEJ mutation. In a third case, both allele 1 and allele 2 can comprise the transgene.

The methods described herein can have several benefits for treating gain of function diseases. As described above, the methods include integrating a transgene into an endogenous gene, followed by, or concurrent with, administration of a silencing agent, where the silencing agent is not present on the transgene. One benefit includes high efficacy correction of the phenotype. The use of a silencing agent together with the transgene can increase the number of cells that exhibit a corrected phenotype (i.e., reduced mutant mRNA or protein and expression of WT protein), beyond the number of cells that have gene editing events correcting the phenotype. Regarding cells that are administered a transgene and silencing agent, the relative increase in the the number cells comprising both reduced mutant mRNA or protein and WT mRNA or protein, can be about 1.5 times more, 2 times more, or 2.5 times more when compared to the number of cells delivered just the transgene. Additional benefits relating to the separate delivery of the silencing agent includes enhanced control of the dosage, reduced toxicity, increased durability and lower off-targeting. Compared to a population of cells with the silencing agent covalently linked to the transgene comprising a partial or full-length coding sequence, cells delivered the silencing agent separate from the transgene can have about 10%, about 20%, about 30%, about 40%, about 50%, about 60%, about 70%, about 80%, about 90%, about 95%, about 99%, reduced toxicity, about 1-5%, about 5-10%, about 10-20%, about 20-30%, about 30-40%, about 40-50%, about 50-60%, about 60-70%, about 70-80%, about 80-90%, about 90-95%, about 95-99% reduced toxicity. By providing the silencing agent on a separate vector, the expression of the silencing construct can be better controlled. In a clinical setting, the use transient gene silencing may be advantageous over continuous production as the dosing schedule (and consequently also the resulting intracellular siRNA concentrations) in a therapeutic regimen can be more easily adapted in relation to therapeutic needs. Additionally, transgenes that are stably integrated in the host genome can be silenced rapidly by histone modifications and hypermethylation of CpG islands in the promoter region. Instead of achieving long-term transgene expression, this chromatin silencing will result in a gradual extinction of transgene activity. Further, long term and continuous expression of high intracellular levels of silencing agents could result in long-term toxicity in mice.

In an embodiment, this document features a transgene with a coding sequence that is resistant to silencing. The coding sequence can be made resistant to silencing by substituting or deleting nucleic acids within the DNA sequence corresponding to the mRNA target site. By way of example, if a transgene comprises a partial corrective coding sequence and encodes exons 10 and 11 of ATXN3 operably linked to a splice acceptor and terminator, and a corresponding shRNA is designed to reduce expression of endogenous ATXN3 genes by targeting the 3′ UTR, then the partial corrective coding sequence is resistant to silencing by not having the endogenous 3′ UTR. By way of another example, if a transgene comprises a partial corrective coding sequence for ATXN3 encoding exons 10 and 11 operably linked to a splice acceptor and terminator, and a corresponding shRNA is designed to reduce expression of endogenous ATXN3 genes by targeting mRNA from exon 11, then the partial corrective coding sequence can be made resistant to silencing by adding synonymous mutations within the partial coding sequence at the corresponding target site.

In some embodiments, the transgenes provided herein for integrating into endogenous genes can comprise several features (e.g., splice acceptor, splice donor, promoter, terminator, coding sequence), but are missing at least one feature that is conventional to a functional gene. By way of example, if the transgene comprises a splice acceptor, coding sequence, and terminator, then the endogenous gene provides the promoter after successful integration.

In some embodiments, this document features methods for using the promoter of an endogenous gene to express a full or partial coding sequence within a transgene. Further, in some embodiments, this document features methods to modify the 3′ end of endogenous genes, where endogenous genes have at least one intron between two coding exons. The intron can be any intron which is removed from precursor messenger RNA by normal messenger RNA processing machinery. The intron can be between 20 bp and >500 kb and comprise elements including a splice donor site, branch sequence, and acceptor site. The transgenes disclosed herein for the modification of the 3′ end of endogenous genes can comprise multiple functional elements, including target sites for rare-cutting endonucleases, homology arms, splice acceptor sequences, coding sequences, and transcription terminators.

In some embodiments, the transgene comprises additional sequences flanking the coding sequence(s) to facilitate integration into the genome. The additional sequences can be ITRs for viral vectors, rare-cutting endonuclease target sites, homology arms, exposed DNA ends, or a combination of ITRs for viral vectors, rare-cutting endonuclease target sites, homology arms and exposed DNA ends. Regarding the rare-cutting endonuclease target sties, the target sites can be a suitable sequence and length for cleavage by a rare-cutting endonuclease. The target site can be amenable to cleavage by CRISPR systems, TAL effector nucleases, zinc-finger nucleases or meganucleases, or a combination of CRISPR systems, TALE nucleases, zinc finger nucleases or meganucleases, or any other site-specific nuclease. The target sites can be positioned such that cleavage by the rare-cutting endonuclease results in liberation of a transgene from a vector. The vector can include viral vectors (e.g., adeno-associated vectors) or non-viral vectors (e.g., plasmids, minicircle vectors, linear DNA). If the transgene comprises two target sites, the target sites can be the same sequence (i.e., targeted by the same rare-cutting endonuclease) or they can be different sequences (i.e., targeted by two or more different rare-cutting endonucleases). If the same nuclease is used to target the endogenous gene, then the target sites within the transgene can be in the same orientation as the endogenous gene (i.e., both in forward directions), or reverse orientation, or a combination of forward or reverse orientations.

In some embodiments, the transgene comprises a first and second target site for one or more rare-cutting endonucleases along with a first and second homology arm. The first and second homology arms can include sequence that is homologous to a genomic sequence at or near the desired site of integration. The homology arms can be a suitable length for participating in homologous recombination with sequence at or near the desired site of integration. The length of each homology arm can be between 20 nt and 10,000 nt (e.g., 20 nt, 30 nt, 40 nt, 50 nt, 100 nt, 200 nt, 300 nt, 400 nt, 500 nt, 600 nt, 700 nt, 800 nt, 900 nt, 1,000 nt, 2,000 nt, 3,000 nt, 4,000 nt, 5,000 nt, 6,000 nt, 7,000 nt, 8,000 nt, 9,000 nt, 10,000 nt). In some embodiments, a homology arm can comprise functional elements, including a target site for a rare-cutting endonuclease and/or a splice acceptor sequence. In some embodiments, a first homology arm (e.g., a left homology arm) can comprise sequence homologous to the intron being targeted, which includes the splice acceptor site of the intron being targeted. In another embodiment, a second homology arm can comprise sequence homologous to genomic sequence downstream of the intron being targeted (e.g., exon sequence, 3′ UTR sequence). However, the second homology arm must not possess splice acceptor functions in the reverse complement direction. To determine if a sequence comprises splice acceptor functions, several steps can be taken, including in silico analysis and experimental tests. To determine if there is potential for splice acceptor functions, the sequence desired for second homology arm can be searched for consensus branch sequences (e.g., YTRAC) and splice acceptor sites (e.g., Y-rich NCAGG). If branch or splice acceptor sequences are present, single nucleotide polymorphisms can be introduced to destroy function, or a different but adjacent sequence not comprising such sequences can be selected. Preferably, the window of sequence that can be used for a second homology arm extends from 1 bp to 10kb downstream of the intron being targeted for integration. To experimentally determine if the second homology possesses splice acceptor function, a synthetic construct comprising the second homology arm within an intron within a reporter gene can be constructed. The construct can then be administered to an appropriate cell type and monitored for splicing function.

In some embodiments, the transgene comprises two splice acceptor sequences, referred to herein as the first and second splice acceptor sequence. The first and second splice acceptor sequences are positioned within the transgene in opposite directions (i.e., in tail-to-tail orientations) and flanking internal sequences (i.e., coding sequences and terminators). When the transgene is integrated into an intron in forward or reverse directions, the splice acceptor sequences facilitate the removal of the adjacent/upstream intron sequence during mRNA processing. The first and second splice acceptor sequences can be the same sequences or different sequences. One or both splice acceptor sequences can be the splice acceptor sequence of the intron where the transgene is to be integrated. One or both splice acceptor sequences can be a synthetic splice acceptor sequence or a splice acceptor sequence from an intron from a different gene.

In some embodiments, the transgene comprises a first and second coding sequence operably linked to the first and second splice acceptor sequences. The first and second coding sequences are positioned within the transgene in opposite directions (i.e., in tail-to-tail orientations). When the transgene is integrated into an endogenous gene in forward or reverse directions, the first or second coding sequence is transcribed into mRNA by the endogenous gene's promoter. The coding sequences can be designed to correct defective coding sequences, introduce mutations, or introduce novel peptide sequences. The first and second coding sequence can be the same nucleic acid sequence and code for the same protein. Alternatively, the first and second coding sequence can be different nucleic acid sequences and code for the same protein (i.e., using the degeneracy of codons). The coding sequence can encode purification tags (e.g., glutathione-S-transferase, poly(His), maltose binding protein, Strep-tag, Myc-tag, AviTag, HA-tag, or chitin binding protein) or reporter proteins (e.g., GFP, RFP, lacZ, cat, luciferase, puro, neomycin). In some embodiments, the transgene comprises a first and second partial coding sequence operably linked to a first and second splice acceptor sequence, and the transgene does not comprise a promoter.

In some embodiments, the transgene can comprise a bidirectional terminator, or a first and second terminator, operably linked to a first and second coding sequence. The bidirectional terminator, or the first and second terminators are positioned within the transgene in opposite directions (i.e., in tail-to-tail orientations). When the transgene is integrated into an endogenous gene in forward or reverse directions, the bidirectional terminator, or first and second terminators, terminate transcription from the endogenous gene's promoter. The first and second terminators can be the same terminators or different terminators.

In some embodiments, this document provides a transgene comprising a first and second rare-cutting endonuclease target site, a first and second splice acceptor sequence, a first and second coding sequence, and one bidirectional terminator or a first and second terminator. The transgene can be integrated in endogenous genes via non-homology dependent methods, including non-homologous end joining and alternative non-homologous end joining or by microhomology-mediated end joining. In one aspect, the transgene is integrated into an intron within the endogenous gene.

In another embodiment, this document provides a transgene comprising a first and second homology arm, a first and second rare-cutting endonuclease target site, a first and second splice acceptor sequence, a first and second coding sequence, and one bidirectional terminator or a first and second terminator. The transgene can be integrated in endogenous genes via both homology dependent methods (e.g., synthesis dependent strand annealing and microhomology-mediated end joining) and non-homology dependent methods (e.g., non-homologous end joining and alternative non-homologous end joining). In one aspect, the transgene is integrated into an intron within the endogenous gene. In another aspect, the transgene is integrated at the end of the intron or the starting of the downstream exon.

In another embodiment, this document provides a transgene comprising a first and second homology arm, a first and second coding sequence, a first and second splice acceptor sequence, and one bidirectional terminator or a first and second terminator. In another embodiment, this document provides a transgene comprising, a first and second coding sequence, a first and second splice acceptor sequence, and one bidirectional terminator or a first and second terminator.

In another embodiment, this document provides a transgene comprising a first and second homology arm, a first and second coding sequence, a first and second splice acceptor sequence, one bidirectional terminator or a first and second terminator, and a first and second additional sequence. In certain embodiments, the additional sequence can be any additional sequence that is present on the transgene at the 5′ and 3′ ends, however, the additional sequence should not comprise any element that functions as a splice acceptor. The additional sequence can be, for example, inverted terminal repeats of a virus genome. The additional sequence can be present on a transgene having a linear format. The linear format permits integration by NHEJ. For example, a transgene may be provided on an adeno-associated virus vector, wherein the additional sequence is the inverted terminal repeats, can be directly integrated by NHEJ at a target site after cleavage by a rare-cutting endonuclease (i.e., no processing of the transgene is required).

In another embodiment, this document provides transgenes within viral vectors, including adeno-associated viruses and adenoviruses, where the transgene comprises a first and second splice acceptor sequence, a first and second coding sequence, and one bidirectional terminator or a first and second terminator.

In another embodiment, this document provides transgenes within viral vectors, including adeno-associated viruses and adenoviruses, where the transgene comprises a first and second homology arm, a first and second splice acceptor sequence, a first and second coding sequence, and one bidirectional terminator or a first and second terminator.

In some embodiments, the transgenes described herein can have a combination of elements including splice acceptors, partial coding sequences, terminators, homology arms, and sites for cleavage by rare-cutting endonucleases. In some embodiments, the combination can be, from 5′ to 3′, [splice acceptor 1]—[coding sequence 1]—[terminator 1]—[terminator 2 RC]—[coding sequence 2 RC]—[splice acceptor 2 RC], where RC stands for reverse complement. This combination may be provided on a linear DNA molecule or AAV molecule and can be integrated by NHEJ through a targeted break in the target gene. In another embodiment, the combination can be, from 5′ to 3′, [rare-cutting endonuclease cleavage site 1]—[splice acceptor 1]—[coding sequence 1]—[terminator 1]—[terminator 2 RC]—[coding sequence 2 RC]—[splice acceptor 2 RC]—[rare-cutting endonuclease cleavage site 1]. In another embodiment, the combination can be, from 5′ to 3′, [rare-cutting endonuclease cleavage site 1]—[homology arm 1]—[splice acceptor 1]—[coding sequence 1]—[terminator 1]—[terminator 2 RC]—[coding sequence 2 RC]—[splice acceptor 2 RC]—[homology arm 2]—[rare-cutting endonuclease cleavage site 2]. In this combination one or more rare-cutting endonucleases can be used to facilitate HR and NHEJ. For example, a single rare-cutting nuclease can cleave the target gene (i.e., a desired intron) and the cleavage sites flanking the homology arms can be designed to be the same target sequence within the intron. In another embodiment, the combination can be, from 5′ to 3′, [homology arm 1 +rare-cutting endonuclease cleavage site 1]—[splice acceptor 1]—[coding sequence 1]—[terminator 1]—[terminator 2 RC]—[coding sequence 2 RC]—[splice acceptor 2 RC]—[homology arm 2]—[rare-cutting endonuclease cleavage site 1]. In this combination, one or more rare-cutting endonucleases can facilitate HR and NHEJ.

In some embodiments, a transgene comprising the structure [rare-cutting endonuclease cleavage site 1]—[homology arm 1]—[splice acceptor 1]—[coding sequence 1]—[terminator 1]—[terminator 2 RC]—[coding sequence 2 RC]—[splice acceptor 2 RC]—[homology arm 2]—[rare-cutting endonuclease cleavage site 2] can be integrated into the DNA through delivery of one or more rare-cutting endonucleases. If one rare-cutting endonuclease is delivered, the rare-cutting endonuclease can liberate the transgene by cleavage at the rare-cutting endonuclease cleavage site 1 and 2. Further, the same rare-cutting endonuclease can create a break within the target gene, simulating insertion through HR or NHEJ.

In other embodiments, a transgene comprising the structure [homology arm 1+rare-cutting endonuclease cleavage site 1]—[splice acceptor 1]—[coding sequence 1]—[terminator 1]—[terminator 2 RC]—[coding sequence 2 RC]—[splice acceptor 2 RC]—[homology arm 2]—[rare-cutting endonuclease cleavage site 1] can be integrated into the DNA thorough delivery of one or more rare-cutting endonucleases. If one rare-cutting endonuclease is delivered, the rare-cutting endonuclease can liberate the transgene by cleavage at the rare-cutting endonuclease cleavage site 1 and 2. Further, the same rare-cutting endonuclease can create a break within the target gene, simulating insertion through HR or NHEJ. Integration by HR can occur when cleavage is upstream of the site of integration (i.e., within a homology arm).

In some embodiments, the location for integration of transgenes can be an intron or an intron-exon junction. When targeting an intron, the partial coding sequence can comprise sequence encoding the peptide produced by the following exons within the endogenous gene. For example, if the transgene is designed to be integrated in intron 9 of an endogenous gene with 11 exons, then the partial coding sequence can comprise sequence encoding the peptide produced by exons 10 and 11 of the endogenous gene. When targeting an intron-exon junction, the transgene can be designed to comprise homology arms with sequence homologous to the 3′ of the intron.

In some embodiments, the coding sequences can be full coding sequences. The full coding sequence can encode an endogenous gene (e.g., Factor VIII, Factor IX, or INS), or reporter genes (e.g., RFP, GFP, cat, lacZ, luciferase). The full coding sequences can be operably linked to splice acceptors and terminators, and placed in a transgene in a tail-to-tail orientation.

In some embodiments, the methods described herein include the use of transgenes comprising a first coding sequence that encodes an amino acid sequence that is homologous to the protein encoded by the endogenous gene or to a polypeptide fragment thereof In some embodiments the encoded amino acid sequence is homologous or identical to a polypeptide fragment of the endogenous gene that is from 5 to 10, from 10 to 20, from 20 to 50, from 50 to 100, from 100 to 200, from 200 to 300, from 300 to 400, from 400 to 500, from 500 to 600, from 600 to 800, from 800 to 1,000, or from 1,000 to 1,200, or more amino acids in length. In some embodiments the encoded amino acid sequence is homologous or identical to a polypeptide fragment of the endogenous gene that is encoded by an exon of the endogenous gene, a partial exon of the endogenous gene, multiple sequential exons of the endogenous gene, a combination of multiple sequential exons and partial exons of the endogenous gene, or the full open reading frame of the endogenous gene. In some embodiments the homology between the amino acid sequence encoded by the first coding sequence of the transgene and the protein or fragment thereof that is encoded by the endogenous gene is at least 30%, 40%, 50%, 60%, 70%, 80%, 90%, 95%, or 99%.

In some embodiments, the methods described herein include the use of transgenes comprising a first and second coding sequence, wherein both coding sequences encode the same amino acid sequence, and wherein the amino acid sequence is homologous to the protein encoded by the endogenous gene or to a polypeptide fragment thereof. In some embodiments the encoded amino acid sequence encoded by the first and second coding sequence is homologous or identical to a polypeptide fragment of the endogenous gene that is from 5 to 10, from 10 to 20, from 20 to 50, from 50 to 100, from 100 to 200, from 200 to 300, from 300 to 400, from 400 to 500, from 500 to 600, from 600 to 800, from 800 to 1,000, or from 1,000 to 1,200, or more amino acids in length. In some embodiments the encoded amino acid sequence is homologous or identical to a polypeptide fragment of the endogenous gene that is encoded by an exon of the endogenous gene, a partial exon of the endogenous gene, multiple sequential exons of the endogenous gene, a combination of multiple sequential exons and partial exons of the endogenous gene, or the full open reading frame of the endogenous gene. In some embodiments the homology between the amino acid sequence encoded by the first and second coding sequences of the transgene and the protein or fragment thereof that is encoded by the endogenous gene is at least 30%, 40%, 50%, 60%, 70%, 80%, 90%, 95%, or 99%. In some embodiments, the genomic modification is the insertion of a transgene in the endogenous CACNA1A genomic sequence. The transgene can include a partial coding sequence for the CACNA1A protein. The partial coding sequence can be homologous to coding sequence within a wild type CACNA1A gene, or a functional variant of the wild type CACNA1A gene, or a mutant of the wild type CACNA1A gene. In some embodiments, the transgene encoding the partial CACNA1A protein is inserted into intron 46 or the beginning of exon 47.

In another embodiment, the genomic modification is the insertion of a transgene in the endogenous ATXN3 genomic sequence. The transgene can include a partial coding sequence for the ATXN3 protein. The partial coding sequence can be homologous to coding sequence within a wild type ATXN3 gene, or a functional variant of the wild type ATXN3 gene, or a mutant of the wild type ATXN3 gene. In some embodiments, the transgene encoding the partial ATXN3 protein is inserted into intron 9 or the beginning of exon 10.

In some embodiments, the methods and compositions described herein can be used to modify the 3′ end of an endogenous gene, thereby resulting in modification of the C-terminus of the protein encoded by the endogenous gene. The modification of the 3′ end of the endogenous gene's coding sequence can include the replacement of the final coding exon (i.e., the exon comprising the stop codon), up to an exon that is between the exon with the start coding and the final exon. As defined herein “replacement” refers to the insertion of DNA in a gene, wherein the inserted DNA provides the information for producing the mRNA and protein of 1 or more exons. Replacement can occur by integrating a transgene into the endogenous gene, wherein the transgene comprises one or more coding sequences operably linked to a splice acceptor. The insertion may or may not result in the deletion of sequence within the endogenous gene (e.g., deletion of introns and exons). For example, if a gene comprises 72 exons, and the start codon is within exon 1, the modification can include replacement of exons 2-72, 3-72, 4-72, 5-72, 6-72, 7-72, 8-72, 9-72, 10-72, 11-72, 12-72, 13-72, 14-72, 15-72, 16-72, 17-72, 18-72, 19-72, 20-72, 21-72, 22-72, or 23-72, or 24-72, or 25-72, or 26-72, or 27-72, or 28-72, or 29-72, or 30-72, or 31-72, or 32-72, or 33-72, or 34-72, or 35-72, or 36-72, or 37-72, or 38-72, or 39-72, or 40-72, or 41-72, or 42-72, or 43-72, or 44-72, or 45-72, or 46-72, or 47-72, or 48-72, or 49-72, or 50-72, or 51-72, or 52-72, or 53-72, or 54-72, or 55-72, or 56-72, or 57-72, or 58-72, or 59-72, or 60-72, or 61-72, or 62-72, or 63-72, or 64-72, or 65-72, or 66-72, or 67-72, or 68-72, or 69-72, or 70-72, or 71-72 or 72. In some embodiments, the endogenous gene's exons can be replaced by integrating a transgene into the endogenous gene, wherein the transgene comprises a first and second partial coding sequence, wherein the first and second partial coding sequence encodes a peptide produced by the endogenous genes exons. For example, the transgene's first and second coding sequence can encode a peptide that is produced by the endogenous gene's exons 2-72, 3-72, 4-72, 5-72, 6-72, 7-72, 8-72, 9-72, 10-72, 11-72, 12-72, 13-72, 14-72, 15-72, 16-72, 17-72, 18-72, 19-72, 20-72, 21-72, 22-72, or 23-72, or 24-72, or 25-72, or 26-72, or 27-72, or 28-72, or 29-72, or 30-72, or 31-72, or 32-72, or 33-72, or 34-72, or 35-72, or 36-72, or 37-72, or 38-72, or 39-72, or 40-72, or 41-72, or 42-72, or 43-72, or 44-72, or 45-72, or 46-72, or 47-72, or 48-72, or 49-72, or 50-72, or 51-72, or 52-72, or 53-72, or 54-72, or 55-72, or 56-72, or 57-72, or 58-72, or 59-72, or 60-72, or 61-72, or 62-72, or 63-72, or 64-72, or 65-72, or 66-72, or 67-72, or 68-72, or 69-72, or 70-72, or 71-72 or 72. The transgene can be integrated within the endogenous gene in the upstream intron or at the beginning of the exon corresponding to the first exon within the transgene's partial coding sequence. The transgene can be designed to be 4.7kb or less, and incorporated into an AAV vector and particle, and delivered in vivo to target cells.

In an embodiment, the transgene is a sequence of DNA that comprises a first and second coding sequence, wherein the partial coding sequences encode a partial protein, wherein the partial protein is homologous to a corresponding region in a functional protein produced from a wild type gene. The host gene or endogenous gene is one in which expression of the protein is aberrant, in other words, is not expressed, is expressed at low levels, or is expressed but the mRNA or protein product or portion thereof is non-functional, has reduced function, or has a gain-of-function, resulting in a disorder in the host.

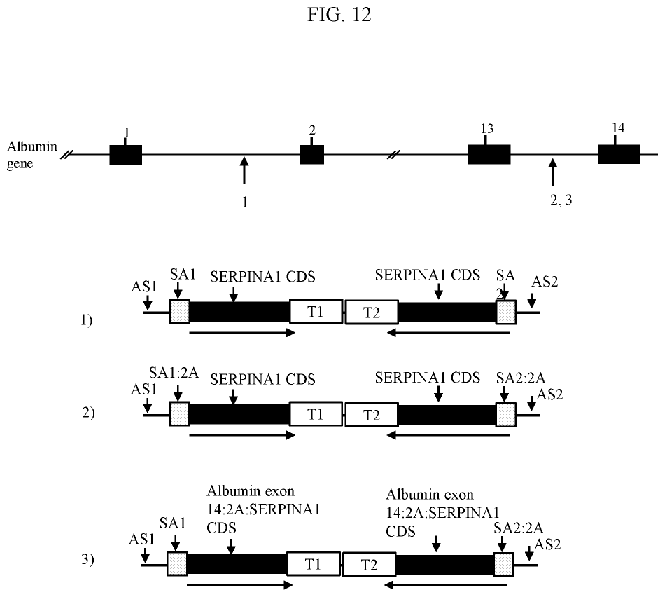

In some embodiments, the transgene comprises a coding sequence for the SERPINA1 gene and the target for insertion is the endogenous SERPINA1 gene. In another embodiment, the target for insertion is the endogenous albumin gene (FIG. 12). In another embodiment, the transgene comprises the SERPINA1 coding sequence without a signal sequence, wherein the SERPINA1 coding sequence is resistant to silencing by an shRNA construct. See, for example, the SERPINA1 coding sequences within the bidirectional transgene in SEQ ID NO:26. In specific embodiments, the shRNA construct can target the sequences GAACUCACCCACGAUAUCAUU (SEQ ID NO:28), GAUGAAGCGUUUAGGCAUGUU (SEQ ID NO:27), CCUAUGAUCUGAAGAGCGUUU (SEQ ID NO:29); UGAUAUCGUGGGUGAGUUCUU (SEQ ID NO:30), CAUGCCUAAACGCUUCAUCUU (SEQ ID NO:31), or ACGCUCUUCAGAUCAUAGGUU (SEQ ID NO:32). In some embodiments, this transgene can be integrated into intron 1 of an albumin gene or intron 1 of a SERPINA1 gene. In another embodiment, the transgene comprises a 2A:SERPINA1 coding sequence with a signal sequence, wherein the SERPINA1 coding sequences are resistant to silencing by the shRNA described within SEQ ID NOs: 27-32. This transgene can be integrated into intron 13 of the albumin gene. In another embodiment, the transgene comprises an albumin exon 14:2A:SERPINA1 coding sequence with a signal sequence, wherein the SERPINA coding sequence is resistant to silencing by the shRNA described within SEQ ID NOs: 27-32. This transgene can be integrated into intron 13 of the albumin gene.

As described herein, the donor molecule can be in a viral or non-viral vector. The vectors can be in the form of circular or linear double-stranded or single stranded DNA. The donor molecule can be conjugated or associated with a reagent that facilitates stability or cellular update. The reagent can be lipids, calcium phosphate, cationic polymers, DEAE-dextran, dendrimers, polyethylene glycol (PEG) cell penetrating peptides, gas-encapsulated microbubbles or magnetic beads. The donor molecule can be incorporated into a viral particle. The virus can be retroviral, adenoviral, adeno-associated vectors (AAV), herpes simplex, pox virus, hybrid adenoviral vector, epstein-bar virus, lentivirus, or herpes simplex virus.

In certain embodiments, the AAV vectors as described herein can be derived from any AAV. In certain embodiments, the AAV vector is derived from the defective and nonpathogenic parvovirus adeno-associated type 2 virus. All such vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3, 1998; Kearns et al., Gene Ther. 9:748-55, 1996). Other AAV serotypes, including AAV1, AAV2, AAV3, AAV4, AAVS, AAV6, AAV7, AAV8, AAV9 and AAVrh.10 and any novel AAV serotype can also be used in accordance with the present methods provided herein. In some embodiments, chimeric AAV is used where the viral origins of the long terminal repeat (LTR) sequences of the viral nucleic acid are heterologous to the viral origin of the capsid sequences. Non-limiting examples include chimeric virus with LTRs derived from AAV2 and capsids derived from AAVS, AAV6, AAV8 or AAV9 (i.e. AAV2/5, AAV2/6, AAV2/8 and AAV2/9, respectively).

In some embodiments, this document features transgenes and methods for modifying the 5′ end of endogenous genes. The transgenes can comprise a first and second promoter, wherein the first promoter is operably linked to a first partial coding sequence, and the second promoter is operably linked to a second partial coding sequence. The first and second partial coding sequences can be operably linked to a first and second splice donor sequence, respectively. The first promoter, first partial coding sequence and first splice donor can be positioned in a head-to-head orientation with the second promoter, second partial coding sequence and second splice donor. This transgene can be integrated into an endogenous gene within an intron or at an exon-intron junction. In some embodiments, the transgenes can be integrated into an endogenous gene using rare-cutting endonucleases. In some embodiments, transgenes comprising a first and second promoter, a first and second partial coding sequence, and a first and second splice donor can be flanked by additional sequence, such as viral inverted terminal repeats (e.g., adeno-associated virus inverted repeats). These transgenes can be integrated into endogenous genes through a targeted double-strand break using a rare-cutting endonuclease.

In another embodiment, transgenes comprising a first and second promoter, a first and second partial coding sequence, and a first and second splice donor are flanked by a first and second rare-cutting endonuclease target site. These transgenes are integrated into endogenous genes through a targeted double-strand break using one or more rare-cutting endonucleases, wherein the one or more rare-cutting endonucleases cleave a sequence within the endogenous gene and cleave the flanking target sites within the transgene.

In another embodiment, transgenes comprising a first and second promoter, a first and second partial coding sequence, and a first and second splice donor are flanked by a first and second homology arm. These transgenes are integrated into endogenous genes through a targeted double-strand break using one or more rare-cutting endonucleases, wherein the one or more rare-cutting endonucleases cleave the endogenous gene.

In another embodiment, transgenes comprising a first and second promoter, a first and second partial coding sequence, and a first and second splice donor are flanked by a first and second homology arm and a first and second rare-cutting endonuclease target site. These transgenes are integrated into endogenous genes through a targeted double-strand break using one or more rare-cutting endonucleases, wherein the one or more rare-cutting endonucleases cleave a sequence within the endogenous gene and cleave the flanking target sites within the transgene. The first and second target sites within the vector flank the first and second homology arm. Alternative, the first target site or second target site, or both the first and second target sites, are be within a homology arm.

In some embodiments, the first and second promoters are replaced with a bidirectional promoter. In other embodiments, the transgenes further comprises a first and second terminator positioned in a tail-to-tail orientation between the first and second promoters. Alternatively, the first and second terminator is substituted with a bidirectional terminator.

In some embodiments, this document features methods for modifying the 5′ end of endogenous genes, wherein the endogenous genes have at least one intron between two coding exons. In some embodiments the intron is any intron which is removed from precursor messenger RNA by normal messenger RNA processing machinery. The intron can be between 20 bp and >500 kb and comprise elements including a splice donor site, branch sequence, and acceptor site. The transgenes disclosed herein for the modification of the 5′ end of endogenous genes can comprise multiple functional elements, including target sites for rare-cutting endonucleases, homology arms, splice acceptor sequences, coding sequences, promoters and transcriptional terminators.

In some embodiments, the methods and compositions described herein are used to modify the 5′ end of an endogenous gene, thereby resulting in modification of the N-terminus of the protein encoded by the endogenous gene. The modification of the 5′ end of the endogenous gene's coding sequence includes the replacement of the first coding exon up to an exon that is between the first exon and the final exon. For example, if a gene comprises 12 exons, the modification can include replacement of exon 1, or 1-2, or 1-3, or 1-4, or 1-5, or 1-6, or 1-7, or 1-8, or 1-9 or 1-10, or 1-11. In some embodiments, the endogenous exons being replaced is replaced with similar sequence. For example, the transgene's first or second coding sequence can comprise exon 1, or 1-2, or 1-3, or 1-4, or 1-5, or 1-6, or 1-7, or 1-8, or 1-9 or 1-10, or 1-11. The transgene is integrated within the endogenous gene in an intron downstream of the exon that is the last exon within the transgene's coding sequence. Alternatively, the transgene is integrated within an exon corresponding to the last exon within the transgene's coding sequence. The transgene is designed to be 4.7kb or less, and incorporated into an AAV vector and particle, and delivered in vivo to target cells.

In some embodiments, the transgene comprises a bidirectional promoter, or a first and second promoter, operably linked to a first and second coding sequence. The bidirectional promoter, or the first and second promoters are positioned within the transgene in opposite directions (i.e., in head-to-head orientations). When the transgene is integrated into an endogenous gene in forward or reverse directions, the bidirectional promoter, or first and second promoters, initiate transcription of the first and second coding sequences. The first and second promoters can be the same promoter or different promoters.

In some embodiments, the transgene can comprise a bidirectional promoter, or a first and second promoter, operably linked to a first and second coding sequence. The bidirectional promoter, or the first and second promoters are positioned within the transgene in opposite directions (i.e., in head-to-head orientations). When the transgene is integrated into an endogenous gene in forward or reverse directions, the bidirectional promoter, or first and second promoters, initiate transcription of the first and second coding sequences. The first and second promoters can be the same promoter or different promoters. The promoters can be, for example, selected from CMV, EF1 alpha, SV40, PGK1, Ubc, human beta actin, CAG, or any promoter with sufficient activity to initiate transcription of the partial coding sequence. Without being bound by theory, the promoter in the reverse direction may cause the creation of double-stranded RNA, thereby resulting in silencing of gene expression upstream of the site of integration. Further, the promoter in forward direction may initiate transcription of RNA that is not subject to the same silencing (e.g., due to codon degeneracy of the coding sequence). Described herein are also methods for reducing potential RNAi from the RNA produced by the promoter in the reverse direction.

In some embodiments, the transgene can comprise a bidirectional terminator, or a first and second terminator between a first and second promoter. The bidirectional terminator, or the first and second terminators are positioned within the transgene in opposite directions (i.e., in tail-to-tail orientations). When the transgene is integrated into an endogenous gene in forward or reverse directions, the bidirectional terminator, or first and second terminators, terminate transcription from the endogenous gene's promoter. The first and second terminators can be the same terminators or different terminators.

In some embodiments, this document provides a transgene comprising a first and second rare-cutting endonuclease target site, a first and second splice donor sequence, a first and second coding sequence, and one bidirectional promoter or a first and second promoter. The transgene can be integrated in endogenous genes via non-homology dependent methods, including non-homologous end joining and alternative non-homologous end joining or by microhomology-mediated end joining. In one aspect, the transgene is integrated into an intron within the endogenous gene.

In another embodiment, this document provides a transgene comprising a first and second homology arm, a first and second rare-cutting endonuclease target site, a first and second splice donor sequence, a first and second coding sequence, and one bidirectional promoter or a first and second promoter. The transgene can be integrated into endogenous genes via both homology dependent methods (e.g., synthesis dependent strand annealing and microhomology-mediated end joining) and non-homology dependent methods (e.g., non-homologous end joining and alternative non-homologous end joining). In one aspect, the transgene is integrated into an intron within the endogenous gene. In another aspect, the transgene is integrated within an exon of the endogenous gene.

In another embodiment, this document provides a transgene comprising a first and second homology arm, a first and second splice donor sequence, a first and second coding sequence, and one bidirectional promoter or a first and second promoter. In another embodiment, this document provides a transgene comprising, a first and second coding sequence, a first and second splice donor sequence, and one bidirectional promoter or a first and second promoter.

In another embodiment, this document provides a transgene comprising a first and second homology arm, a first and second coding sequence, a first and second splice donor sequence, one bidirectional terminator or a first and second terminator, and a first and second additional sequence. The additional sequence can be any additional sequence that is present on the transgene at the 5′ and 3′ ends, however, the additional sequence should not comprise any element that functions as a splice acceptor or splice donor. The additional sequence can be, for example, inverted terminal repeats of an adeno-associated virus genome, or left and right transposon ends.

In another aspect, the transgene for integration can be designed to integrate through multiple repair pathways while creating a desired effect with each outcome. By way of example, a transgene can comprise a first and second arm homology arm, a first and second rare-cutting endonuclease target site, a first and second coding sequence, a first and second promoter, and may be provided within an AAV genome (i.e., flanked by 145 nucleotide inverted terminal repeats). Following expression by a rare-cutting endonuclease, the following outcomes can occur: 1) integration of the entire AAV genome at the target site by NHEJ in either forward or reverse orientation, 2) integration of the sequence between the first and second rare-cutting endonuclease target sites at the target site by NHEJ in either forward or reverse orientation, 3) integration by HR using the first and second homology arms, or 4) any combination of the above outcomes. Following integration with any of the above-mentioned outcomes, the transgene described herein can correct or alter the protein sequence produced by the endogenous gene. By way of another example, a transgene can comprise a first and second splice acceptor, a first and second coding sequence, a first and second terminator and may be provided within an AAV genome. Following expression by a rare-cutting endonuclease, the following outcomes can occur: 1) integration of the entire AAV in the forward orientation, 2) integration of the entire AAV in the reverse direction, or 3) concatomerization of vectors in the forward, reverse or both forward and reverse directions.

In some embodiments, the combination can be, from 5′ to 3′, [splice donor 1 RC]—[partial coding sequence 1 RC]—[promoter 1 RC]—[promoter 2]—[partial coding sequence 2]—[splice donor 2], where RC stands for reverse complement. This combination may be provided on a linear DNA molecule or AAV molecule and can be integrated by NHEJ through a targeted break in the target gene.

In another embodiment, the combination can be, from 5′ to 3′, [rare-cutting endonuclease cleavage site 1]—[splice donor 1 RC]—[partial coding sequence 1 RC]—[promoter 1 RC]—[promoter 2]—[partial coding sequence 2]—[splice donor 2]—[rare-cutting endonuclease cleavage site 2].

In another embodiment, the combination can be, from 5′ to 3′, [rare-cutting endonuclease cleavage site 1]—[homology arm 1]—[splice donor 1 RC]—[partial coding sequence 1 RC]—[promoter 1 RC]—[promoter 2]—[partial coding sequence 2]—[splice donor 2]—[homology arm 2]—[rare-cutting endonuclease cleavage site 2]. In this combination one or more rare-cutting endonucleases can be used to facilitate HR and NHEJ. For example, a single rare-cutting nuclease can cleave the target gene (i.e., a desired intron) and the cleavage sites flanking the homology arms can be designed to be the same target sequence within the intron.

In another embodiment, the combination can be, from 5′ to 3′, [homology arm 1+rare-cutting endonuclease cleavage site 1]—[splice donor 1 RC]—[partial coding sequence 1 RC]—[promoter 1 RC]—[promoter 2]—[partial coding sequence 2]—[splice donor 2]—[homology arm 2]—[rare-cutting endonuclease cleavage site 2]. In this combination, one or more rare-cutting endonucleases can facilitate HR and NHEJ. For example, a single-rare cutting nuclease can cleave within homology arm 1, downstream of homology arm 2, and at the genomic target site (i.e., at the site with homology to the sequence in the homology arm 1).

In another embodiment, the combination can be from 5′ to 3′, [left end for a transposase]—[splice donor 1 RC]—[partial coding sequence 1 RC]—[promoter 1 RC]—[promoter 2]—[partial coding sequence 2]—[splice donor 2]—[right end for a transposase]. In all embodiments, the splice donor 1 and splice donor 2 can be the same or different sequences; the partial coding sequence 1 and partial coding sequence 2 can be the same or different sequences; the promoter 1 and promoter 2 can be the same or different sequences.

In some embodiments, a transgene comprising the structure [rare-cutting endonuclease cleavage site 1]—[homology arm 1]—[splice donor 1 RC]—[partial coding sequence 1]—[promoter 1 RC]—[promoter 2]—[partial coding sequence 2]—[splice donor 2]—[homology arm 2]—[rare-cutting endonuclease cleavage site 2] can be integrated into the DNA through delivery of one or more rare-cutting endonucleases. If one rare-cutting endonuclease is delivered, the rare-cutting endonuclease can liberate the transgene by cleavage at the rare-cutting endonuclease cleavage site 1 and 2. Further, the same rare-cutting endonuclease can create a break within the target gene, simulating insertion through HR or NHEJ.

In other embodiments, a transgene comprising the structure [homology arm 1+rare-cutting endonuclease cleavage site 1]—[splice donor 1 RC]—[partial coding sequence 1]—[promoter 1 RC]—[promoter 2]—[partial coding sequence 2]—[splice donor 2]—[homology arm 2]—[rare-cutting endonuclease cleavage site 1] can be integrated into the DNA thorough delivery of one or more rare-cutting endonucleases. If one rare-cutting endonuclease is delivered, the rare-cutting endonuclease can liberate the transgene by cleavage at the rare-cutting endonuclease cleavage site 1 and 2. Further, the same rare-cutting endonuclease can create a break within the target gene, simulating insertion through HR or NHEJ. Integration by HR can occur when cleavage is upstream of the site of integration (i.e., within a homology arm).

In some embodiments, the partial coding sequences can be codon adjusted. The codon adjustment can be designed to 1) reducing double-stranded RNA and 2) optimizing protein expression. If a transgene comprising a first and second partial coding sequence operably linked to a first and second promoter is integrated into an endogenous gene, and the first and second partial coding sequences are homologous to each other and the endogenous gene, then double-stranded RNA may be produced. The partial coding sequences can be codon adjusted to minimize RNA pairing. In some embodiments, the codon optimization can be complete and different for the first and second partial coding sequences. For example, partial coding sequence 1 can have a different nucleotide sequence than partial coding sequence 2, and both partial coding sequences 1 and 2 can be a different sequence than the corresponding sequence within the endogenous gene-of-interest.

In another embodiment, the codon optimization can be split between the first and second partial coding sequences. For example, the first partial coding sequence can have a mixture of non-codon adjusted sequence (i.e., homologous to the corresponding sequence within the endogenous gene-of-interest) and codon adjusted sequence. In this example, the second partial coding sequence can have the opposite adjustment. For example, within a 200 nucleotide partial coding sequence 1 and 2, the nucleotides 1-100 of partial coding sequence 1 can be homologous to the sequence within the endogenous gene-of-interest, and the nucleotides 101-200 can be codon adjusted to have minimal sequence similarities to the endogenous gene-of-interest; the nucleotides 1-100 of partial coding sequence 2 can be codon adjusted to have minimal sequence similarities to the endogenous gene-of-interest, and nucleotides 101-200 can be homologous to the sequence within the endogenous gene-of-interest.

In some embodiments, the transgenes described herein can comprise a first and second coding sequence encoding an amino acid sequence that is homologous to an amino acid sequence encoded by an endogenous gene. The coding sequences can be in tail-to-tail orientation and encode the same amino acids. By way of example, a cell can comprise an endogenous gene with 10 exons and 9 introns. A transgene with a first and second coding sequence can encode the amino acids produced by exon 10 of the endogenous gene, and the transgene can be integrated into intron 9. A transgene with a first and second coding sequence can encode the amino acids produced by exon 9 and 10 of the endogenous gene, and the transgene can be integrated into intron 8. A transgene with a first and second coding sequence can encode the amino acids produced by exon 8, 9 and 10 of the endogenous gene, and the transgene can be integrated into intron 7. A transgene with a first and second coding sequence can encode the amino acids produced by exon 7, 8, 9 and 10 of the endogenous gene, and the transgene can be integrated into intron 6. A transgene with a first and second coding sequence can encode the amino acids produced by exon 6, 7, 8, 9 and 10 of the endogenous gene, and the transgene can be integrated into intron 5. A transgene with a first and second coding sequence can encode the amino acids produced by exon 5, 6, 7, 8, 9 and 10 of the endogenous gene, and the transgene can be integrated into intron 4. A transgene with a first and second coding sequence can encode the amino acids produced by exon 4, 5, 6, 7, 8, 9 and 10 of the endogenous gene, and the transgene can be integrated into intron 3. A transgene with a first and second coding sequence can encode the amino acids produced by exon 3, 4, 5, 6, 7, 8, 9 and 10 of the endogenous gene, and the transgene can be integrated into intron 2. A transgene with a first and second coding sequence can encode the amino acids produced by exon 2, 3, 4, 5, 6, 7, 8, 9 and 10 of the endogenous gene, and the transgene can be integrated into intron 1.

In some embodiments, the transgenes described herein can comprise a first and second coding sequence encoding the same amino acid sequence but different nucleic acid sequences. Using the degeneracy of codons, the first coding sequence can differ from the second coding sequence. The first coding sequence can have 60%, 70%, 80%, 90%, 95%, or 99% nucleotide homology with the second coding sequence. The first coding sequence can have between 60% to 70%, 70% to 80%, 80% to 90%, 90% to 99% nucleotide homology with the second coding sequence.

In some embodiments, the transgenes described herein which encode amino acids with homology to a first endogenous gene can be integrated in a second endogenous gene. The second endogenous gene can be a gene that has a promoter of interest for expressing the transgene. The genes can be, for example, F9, albumin, CFHR2, FGA, HPX, C9, or F2. The transgene can be integrated within an intron of the second endogenous gene. In some embodiments, the transgene can encode from 5′ to 3′, [2A]-[full-length CDS from first endogenous gene]. In some embodiments, the transgene can encode from 5′ to 3′, [full-length CDS from first endogenous gene]. In some embodiments, the transgene can encode from 5′ to 3′, [partial CDS from first endogenous gene]. In some embodiments, the transgene can be integrated into intron 1 of the albumin gene and the transgene can comprise from 5′ to 3′, [SA]-[partial CDS from SERPINA1]. In some embodiments, the transgene can encode amino acids in addition to the amino acids with homology to amino acids produced by a first endogenous gene. The additional amino acids can be the amino acids of the second endogenous gene encoded by the exons downstream of the intron where the transgene is integrated. This partial coding sequence can be fused to a 2A sequence which is then fused to the amino acids from the first endogenous gene. In some embodiments, the transgene can encode from 5′ to 3′, [partial CDS from second endogenous gene]-[2A]-[full-length CDS from first endogenous gene]. In other embodiments, the transgene can encode from 5′ to 3′, [partial CDS from second endogenous gene]-[2A]-[full-length CDS from first endogenous gene]. In some embodiments, the transgene can be integrated into intron 13 of the albumin gene and the transgene can comprise from 5′ to 3′ [SA]-[albumin exon 14 CDS]-[2A]-[full-length CDS from first endogenous gene].

In some embodiments, the genomic modification is the insertion of a transgene in the endogenous ATXN2 genomic sequence. The transgene can include a partial coding sequence for the ATXN2 protein. The partial coding sequence can be homologous to coding sequence within a wild type ATXN2 gene, or a functional variant of the wild type ATXN2 gene, a codon adjusted version of the ATXN2 gene, or a mutant ATXN2 gene. In some embodiments, the transgene encoding the partial ATXN2 protein is inserted into intron 1 of the endogenous ATXN2 gene.

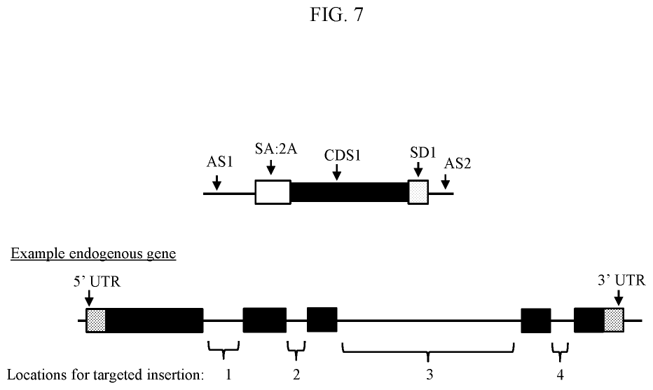

In some embodiments, the transgenes provided herein comprises a first and second partial coding sequence encoding the peptide produced by exon 1 of the ATXN2 gene (FIG. 7). The transgenes can be integrated within the endogenous ATXN2 gene within intron 1 or at the exon 1 intron 1 junction. This embodiment is particularly useful in cells comprising an expanded trinucleotide repeat in exon 1 of ATXN2.

The methods and compositions provided herein can be used to modify genes encoding proteins within cells. The endogenous proteins can include, fibrinogen, prothrombin, tissue factor, Factor V, Factor VII, Factor VIII, Factor IX, Factor X, Factor XI, Factor XII (Hageman factor), Factor XIII (fibrin-stabilizing factor), von Willebrand factor, prekallikrein, high molecular weight kininogen (Fitzgerald factor), fibronectin, antithrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, glucocerebrosidase (GBA), a-galactosidase A (GLA), iduronate sulfatase (IDS), iduronidase (IDUA), acid sphingomyelinase (SMPD1), MMAA, MMAB, MMACHC, MMADHC (C2orf25), MTRR, LMBRD1, MTR, propionyl-CoA carboxylase (PCC) (PCCA and/or PCCB subunits), a glucose-6-phosphate transporter (G6PT) protein or glucose-6-phosphatase (G6Pase), an LDL receptor (LDLR), ApoB, LDLRAP-1, a PCSK9, a mitochondrial protein such as NAGS (N-acetylglutamate synthetase), CPS1 (carbamoyl phosphate synthetase I), and OTC (ornithine transcarbamylase), ASS (argininosuccinic acid synthetase), ASL (argininosuccinase acid lyase) and/or ARG1 (arginase), and/or a solute carrier family 25 (SLC25A13, an aspartate/glutamate carrier) protein, a UGT1A1 or UDP glucuronsyltransferase polypeptide Al, a fumarylacetoacetate hydrolyase (FAH), an alanine-glyoxylate aminotransferase (AGXT) protein, a glyoxylate reductase/hydroxypyruvate reductase (GRHPR) protein, a transthyretin gene (TTR) protein, an ATP7B protein, a phenylalanine hydroxylase (PAH) protein, an USH2A protein, an ATXN protein, and a lipoprotein lyase (LPL) protein.

The transgene can include sequence for modifying an endogenous gene that comprises a loss-of-function or gain-of-function mutation. The mutation can include those that result in the following genetic diseases: achondroplasia, achromatopsia, acid maltase deficiency, adenosine deaminase deficiency, adrenoleukodystrophy, aicardi syndrome, alpha-1 antitrypsin deficiency, alpha-thalassemia, androgen insensitivity syndrome, pert syndrome, arrhythmogenic right ventricular dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, generalized gangliosidoses (e.g., GM1), hemochromatosis, the hemoglobin C mutation in the 6th codon of beta-globin (HbC), hemophilia, Huntington's disease, hypophosphatasia, Klinefleter syndrome, Krabbes Disease, Langer-Giedion Syndrome, leukocyte adhesion deficiency, leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome, lysosomal storage diseases (e.g., Gaucher's disease, GM1, Fabry disease and Tay-Sachs disease), von Willebrand disease, usher syndrome, polycystic kidney disease, spinocerebellar ataxia type 2, spinal and bulbar muscular atrophy, Friedreich's ataxia, and myotonic dystrophy type 2.

In some embodiments, the transgene can include sequence for modifying an endogenous gene for correction of alpha-1 antitrypsin deficiency. The transgene can comprise coding sequence for the SERPINA1 gene and can be integrated into the endogenous SERPINA1 gene or the albumin gene.

As described herein, the transgenes may be provided on a viral or non-viral vector. The vectors can be in the form of circular or linear double-stranded or single stranded DNA. The donor molecule can be conjugated or associated with a reagent that facilitates stability or cellular update. The reagent can be lipids, calcium phosphate, cationic polymers, DEAE-dextran, dendrimers, polyethylene glycol (PEG) cell penetrating peptides, gas-encapsulated microbubbles or magnetic beads. The donor molecule can be incorporated into a viral particle. The virus can be retroviral, adenoviral, adeno-associated vectors (AAV), herpes simplex, pox virus, hybrid adenoviral vector, epstein-bar virus, lentivirus, or herpes simplex virus.

In another embodiment, the methods described herein can include administration of a silencing agent to cells. The silencing agent is designed to reduce the expression of an endogenous gene, but not the modified gene produced by integration of the transgene. The silencing sequence can be administered in trans with the transgene at the same time or following administration of the transgene. The nucleic acid sequence can be in a format capable of inducing gene silencing within a target nucleic acid (e.g., microRNA, hairpin RNA, antisense RNA, double stranded RNA, or antisense DNA). The nucleic acid sequence can be targeted to different regions in the endogenous gene's mRNA, including the 5′ UTR, coding sequence, or 3′ UTR.

In some embodiments, this document describes methods to modify an endogenous gene by integrating a transgene with coding sequence resistant to silencing, and administering a silencing agent that reduces expression of the endogenous gene corresponding to the sequence carried within the transgene's coding sequence. Any method can be used to administer a silencing agent described herein. For example, in mammals, administration can be direct; oral; or parenteral (e.g., by subcutaneous, intraventricular, intramuscular, or intraperitoneal injection, or by intravenous drip). Administration can be rapid (e.g., by injection), or can occur over a period of time (e.g., by slow infusion or administration of slow release formulations). In cell culture, administration can be direct (e.g., electroporation) or through carriers such as lipid nanoparticles or magnetic nanoparticles.

When administering, for example dsRNA, to mammals for example, the silencing agent may be conjugated or unconjugate or formulated with or without liposomes, and can be administered to a patient. For such, a dsRNA molecule can be formulated into compositions such as sterile and non-sterile aqueous solutions, non-aqueous solutions in common solvents such as alcohols, or solutions in liquid or solid oil bases. Such solutions also can contain buffers, diluents, and other suitable additives. For parenteral, intrathecal, or intraventricular administration, a dsRNA molecule can be formulated into compositions such as sterile aqueous solutions, which also can contain buffers, diluents, and other suitable additives (e.g., penetration enhancers, carrier compounds, and other pharmaceutically acceptable carriers).

In addition, silencing agents can be administered to a mammal as biologic or abiologic means. Abiologic delivery can be accomplished by a variety of methods including, without limitation, (1) loading liposomes with an RNA or DNA molecule provided herein and (2) complexing an RNA or DNA molecule with lipids or liposomes to form nucleic acid-lipid or nucleic acid-liposome complexes. The liposome can be composed of cationic and neutral lipids commonly used to transfect cells in vitro. Cationic lipids can complex (e.g., charge-associate) with negatively charged nucleic acids to form liposomes. Examples of cationic liposomes include, without limitation, lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene® (Qiagen, Valencia, Calif). In addition, systemic delivery methods can be optimized using commercially available cationic lipids such as DDAB or DOTAP, each of which can be mixed with a neutral lipid such as DOPE or cholesterol.

Biologic delivery can be accomplished by a variety of methods including, without limitation, the use of viral vectors. For example, viral vectors (e.g., adenovirus, AAV and herpesvirus vectors) can be used to deliver dsRNA molecules to liver cells. Standard molecular biology techniques can be used to introduce one or more of the silencing agents provided herein into one of the many different viral vectors previously developed to deliver nucleic acid to cells. These resulting viral vectors can be used to deliver the one or more silencing agents to cells by, for example, infection.

Silencing agents described herein can be formulated in a pharmaceutically acceptable carrier or diluent. A “pharmaceutically acceptable carrier” (also referred to herein as an “excipient”) is a pharmaceutically acceptable solvent, suspending agent, or any other pharmacologically inert vehicle. Pharmaceutically acceptable carriers can be liquid or solid, and can be selected with the planned manner of administration in mind so as to provide for the desired bulk, consistency, and other pertinent transport and chemical properties. Typical pharmaceutically acceptable carriers include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

The methods and compositions described herein are applicable to any eukaryotic organism in which it is desired to alter the organism through genomic modification. The eukaryotic organisms include plants, algae, animals, fungi and protists. The eukaryotic organisms can also include plant cells, algae cells, animal cells, fungal cells and protist cells.

Exemplary mammalian cells include, but are not limited to, oocytes, K562 cells, CHO (Chinese hamster ovary) cells, HEP-G2 cells, BaF-3 cells, Schneider cells, COS cells (monkey kidney cells expressing SV40 T-antigen), CV-1 cells, HuTu80 cells, NTERA2 cells, NB4 cells, HL-60 cells and HeLa cells, 293 cells (see, e.g., Graham et al. (1977) J. Gen. Virol. 36:59), and myeloma cells like SP2 or NSO (see, e.g., Galfre and Milstein (1981) Meth. Enzymol. 73(B):3 46). Peripheral blood mononucleocytes (PBMCs) or T-cells can also be used, as can embryonic and adult stem cells. For example, stem cells that can be used include embryonic stem cells (ES), induced pluripotent stem cells (iPSC), mesenchymal stem cells, hematopoietic stem cells, liver stem cells, skin stem cells and neuronal stem cells.

The methods and compositions of the methods provided herein can be used in the production of modified organisms. The modified organisms can be small mammals, companion animals, livestock, and primates. Non-limiting examples of rodents may include mice, rats, hamsters, gerbils, and guinea pigs. Non-limiting examples of companion animals may include cats, dogs, rabbits, hedgehogs, and ferrets. Non-limiting examples of livestock may include horses, goats, sheep, swine, llamas, alpacas, and cattle. Non-limiting examples of primates may include capuchin monkeys, chimpanzees, lemurs, macaques, marmosets, tamarins, spider monkeys, squirrel monkeys, and vervet monkeys. The methods and compositions of the methods provided herein can be used in humans.

Exemplary plants and plant cells which can be modified using the methods described herein include, but are not limited to, monocotyledonous plants (e.g., wheat, maize, rice, millet, barley, sugarcane), dicotyledonous plants (e.g., soybean, potato, tomato, alfalfa), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); vegetative crops for consumption (e.g. soybean and other legumes, squash, peppers, eggplant, celery etc), flowering plants (e.g., petunia, rose, chrysanthemum), conifers and pine trees (e.g., pine fir, spruce); poplar trees (e.g. P. tremulaxP. alba); fiber crops (cotton, jute, flax, bamboo) plants used in phytoremediation (e.g., heavy metal accumulating plants); oil crops (e.g., sunflower, rape seed) and plants used for experimental purposes (e.g., Arabidopsis). The methods disclosed herein can be used within the genera Asparagus, Avena, Brassica, Citrus, Citrullus, Capsicum, Cucurbita, Daucus, Erigeron, Glycine, Gossypium, Hordeum, Lactuca, Lolium, Lycopersicon, Malus, Manihot, Nicotiana, Orychophragmus, Oryza, Persea, Phaseolus, Pisum, Pyrus, Prunus, Raphanus, Secale,

Solanum, Sorghum, Triticum, Vitis, Vigna, and Zea. The term plant cells include isolated plant cells as well as whole plants or portions of whole plants such as seeds, callus, leaves, and roots. The present disclosure also encompasses seeds of the plants described above wherein the seed has the has been modified using the compositions and/or methods described herein. The present disclosure further encompasses the progeny, clones, cell lines or cells of the transgenic plants described above wherein the progeny, clone, cell line or cell has the transgene or gene construct. Exemplary algae species include microalgae, diatoms, Botryococcus braunii, Chlorella, Dunaliella tertiolecta, Gracileria, Pleurochrysis carterae, Sorgassum and Ulva.

The methods described in this document can include the use of rare-cutting endonucleases for stimulating homologous recombination or non-homologous integration of a transgene molecule into an endogenous gene. The rare-cutting endonuclease can include CRISPR, TALENs, or zinc-finger nucleases (ZFNs). The CRISPR system can include CRISPR/Cas9 or CRISPR/Cas12a (Cpfl). The CRISPR system can include variants which display broad PAM capability (Hu et al., Nature 556, 57-63, 2018; Nishimasu et al., Science DOI: 10.1126, 2018) or higher on-target binding or cleavage activity (Kleinstiver et al., Nature 529:490-495, 2016). The gene editing reagent can be in the format of a nuclease (Mali et al., Science 339:823-826, 2013; Christian et al., Genetics 186:757-761, 2010), nickase (Cong et al., Science 339:819-823, 2013; Wu et al., Biochemical and Biophysical Research Communications 1:261-266, 2014), CRISPR-FokI dimers (Tsai et al., Nature Biotechnology 32:569-576, 2014), or paired CRISPR nickases (Ran et al., Cell 154:1380-1389, 2013).

Genome editing generally refers to the process of modifying the nucleotide sequence of a genome, preferably in a precise or pre-determined manner. Examples of methods of genome editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HR) and non-homologous end joining (NHEJ), as recently reviewed in Cox et al., Nature Medicine 21(2), 121-31 (2015). These two main DNA repair processes consist of a family of alternative pathways. NHEJ directly joins the DNA ends resulting from a double-strand break, sometimes with the loss or addition of nucleotide sequence, which may disrupt or enhance gene expression. HR utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few basepairs flanking the DNA break site to drive a more favored DNA end joining repair outcome, and recent reports have further elucidated the molecular mechanism of this process; see, e.g., Cho and Greenberg, Nature 518, 174-76 (2015); Kent et al., Nature Structural and Molecular Biology, Adv. Online doi:10.1038/nsmb.2961(2015); Mateos-Gomez et al., Nature 518, 254-57 (2015); Ceccaldi et al., Nature 528, 258-62 (2015).

CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the CRISPR locus, expression of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary structures (e.g., hairpins) and/or comprise unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA comprises a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also comprises polynucleotide sequences encoding CRISPR Associated (Cas) genes. Cas genes encode endonucleases involved in the biogenesis and the interference stages of crRNA function in prokaryotes. Some Cas genes comprise homologous secondary and/or tertiary structures.

crRNA biogenesis in a Type II CRISPR system in nature requires a trans-activating CRISPR RNA (tracrRNA). The tracrRNA can be modified by endogenous RNaseIII, and then hybridizes to a crRNA repeat in the pre-crRNA array. Endogenous RNaseIII can be recruited to cleave the pre-crRNA. Cleaved crRNAs can be subjected to exoribonuclease trimming to produce the mature crRNA form (e.g., 5′ trimming). The tracrRNA can remain hybridized to the crRNA, and the tracrRNA and the crRNA associate with a rare-cutting endonuclease (e.g., Cas9). The crRNA of the crRNA-tracrRNA-Cas9 complex can guide the complex to a target nucleic acid to which the crRNA can hybridize. Hybridization of the crRNA to the target nucleic acid can activate Cas9 for targeted nucleic acid cleavage. The target nucleic acid in a Type II CRISPR system is referred to as a protospacer adjacent motif (PAM). In nature, the PAM is essential to facilitate binding of a rare-cutting endonuclease (e.g., Cas9) to the target nucleic acid. Type II systems (also referred to as Nmeni or CASS4) are further subdivided into Type II-A (CASS4) and II-B (CASS4a). Jinek et al., Science, 337(6096):816-821 (2012) showed that the CRISPR/Cas9 system is useful for RNA-programmable genome editing, and international patent application publication number WO2013/176772 provides numerous examples and applications of the CRISPR/Cas endonuclease system for site-specific gene editing.

Type V CRISPR systems have several important differences from Type II systems. For example, Cpfl is a single RNA-guided endonuclease that, in contrast to Type II systems, lacks tracrRNA. In fact, Cpfl-associated CRISPR arrays can be processed into mature crRNAs without the requirement of an additional trans-activating tracrRNA. The Type V CRISPR array can be processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems can start with 20-24 nucleotides of spacer sequence followed by about 22 nucleotides of direct repeat. Also, Cpfl can utilize a T-rich protospacer-adjacent motif such that Cpfl-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpfl cleaves DNA via a staggered DNA double-stranded break with a 4 or 5 nucleotide 5′ overhang. Type II systems cleave via a blunt double-stranded break. Similar to Type II systems, Cpfl contains a predicted RuvC-like endonuclease domain, but lacks a second HNH endonuclease domain, which is in contrast to Type II systems.

Exemplary CRISPR/Cas polypeptides include the Cas9 polypeptides as published in Fonfara et al., Nucleic Acids Research, 42: 2577-2590 (2014). Fonfara et al., also provides PAM sequences for the Cas9 polypeptides from various species.

A rare-cutting endonuclease is a nuclease used in genome editing to cleave DNA. The rare-cutting endonuclease can be administered to a cell or a patient as either: one or more polypeptides, or one or more mRNAs encoding the polypeptide. Any of the enzymes or orthologs disclosed herein may be utilized in the methods herein.

In the context of a CRISPR/Cas9 or CRISPR/Cpfl system, the rare-cutting endonuclease can bind to a guide RNA that, in turn, specifies the site in the target DNA to which the polypeptide is directed. In the CRISPR/Cas9 or CRISPR/Cpfl systems disclosed herein, the rare-cutting endonuclease can be an endonuclease, such as a DNA endonuclease.

A rare-cutting endonuclease can comprise a plurality of nucleic acid-cleaving (i.e., nuclease) domains. Two or more nucleic acid-cleaving domains can be linked together via a linker. For example, the linker can comprise a flexible linker. Linkers can comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length.

Naturally-occurring wild-type Cas9 enzymes comprise two nuclease domains, a HNH nuclease domain and a RuvC domain. Herein, the term “Cas9” refers to both a naturally-occurring and a recombinant Cas9. Cas9 enzymes contemplated herein can comprise a HNH or HNH-like nuclease domain, and/or a RuvC or RuvC-like nuclease domain.

HNH or HNH-like domains comprise a McrA-like fold. HNH or HNH-like domains comprises two antiparallel (3-strands and an a-helix. HNH or HNH-like domains comprises a metal binding site (e.g., a divalent cation binding site). HNH or HNH-like domains can cleave one strand of a target nucleic acid (e.g., the complementary strand of the crRNA targeted strand).

RuvC or RuvC-like domains comprise an RNaseH or RNaseH-like fold. RuvC/RNaseH domains are involved in a diverse set of nucleic acid-based functions including acting on both RNA and DNA. The RNaseH domain comprises 5 β-strands surrounded by a plurality of α-helices. RuvC/RNaseH or RuvC/RNaseH-like domains comprise a metal binding site (e.g., a divalent cation binding site). RuvC/RNaseH or

RuvC/RNaseH-like domains can cleave one strand of a target nucleic acid (e.g., the non-complementary strand of a double-stranded target DNA).

rare-cutting endonucleases can introduce double-strand breaks or single-strand breaks in nucleic acids, e.g., genomic DNA. The double-strand break can stimulate a cell's endogenous DNA-repair pathways (e.g., homology-dependent repair (HR) or NHEJ or alternative non-homologous end joining (A-NHEJ) or microhomology-mediated end joining (MMEJ)). NHEJ can repair cleaved target nucleic acid without the need for a homologous template. This can sometimes result in small deletions or insertions (indels) in the target nucleic acid at the site of cleavage, and can lead to disruption or alteration of gene expression. HR can occur when a homologous repair template, or donor, is available. The homologous donor template can comprise sequences that are homologous to sequences flanking the target nucleic acid cleavage site. The sister chromatid can be used by the cell as the repair template. However, for the purposes of genome editing, the repair template can be supplied as an exogenous nucleic acid, such as a plasmid, duplex oligonucleotide, single-strand oligonucleotide or viral nucleic acid. With exogenous donor templates, an additional nucleic acid sequence (such as a transgene) or modification (such as a single or multiple base change or a deletion) can be introduced between the flanking regions of homology so that the additional or altered nucleic acid sequence also becomes incorporated into the target locus. MMEJ can result in a genetic outcome that is similar to NHEJ in that small deletions and insertions can occur at the cleavage site. MMEJ can make use of homologous sequences of a few basepairs flanking the cleavage site to drive a favored end-joining DNA repair outcome. In some instances, it may be possible to predict likely repair outcomes based on analysis of potential microhomologies in the nuclease target regions.

Thus, in some cases, homologous recombination can be used to insert an exogenous polynucleotide sequence into the target nucleic acid cleavage site. An exogenous polynucleotide sequence is termed a “donor polynucleotide” (or donor or donor sequence, or transgene) herein. The donor polynucleotide, a portion of the donor polynucleotide, a copy of the donor polynucleotide, or a portion of a copy of the donor polynucleotide can be inserted into the target nucleic acid cleavage site. The donor polynucleotide can be an exogenous polynucleotide sequence, i.e., a sequence that does not naturally occur at the target nucleic acid cleavage site.

The modifications of the target DNA due to NHEJ and/or HR can lead to, for example, mutations, deletions, alterations, integrations, gene correction, gene replacement, gene tagging, transgene insertion, nucleotide deletion, gene disruption, translocations and/or gene mutation. The processes of deleting genomic DNA and integrating non-native nucleic acid into genomic DNA are examples of genome editing.

The rare-cutting endonuclease can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary rare-cutting endonuclease [e.g., Cas9 from S. pyogenes, US2014/0068797 Sequence ID No. 8 or Sapranauskas et al., Nucleic Acids Res, 39(21): 9275-9282 (2011)], and various other rare-cutting endonuclease. The rare-cutting endonuclease can comprise at least 70, 75, 80, 85, 90, 95, 97, 99, or 100% identity to a wild-type rare-cutting endonuclease (e.g., Cas9 from S. pyogenes, supra) over 10 contiguous amino acids. The rare-cutting endonuclease can comprise at most: 70, 75, 80, 85, 90, 95, 97, 99, or 100% identity to a wild-type rare-cutting endonuclease (e.g., Cas9 from S. pyogenes, supra) over 10 contiguous amino acids. The rare-cutting endonuclease can comprise at least: 70, 75, 80, 85, 90, 95, 97, 99, or 100% identity to a wild-type rare-cutting endonuclease (e.g., Cas9 from S. pyogenes, supra) over 10 contiguous amino acids in a HNH nuclease domain of the rare-cutting endonuclease. The rare-cutting endonuclease can comprise at most: 70, 75, 80, 85, 90, 95, 97, 99, or 100% identity to a wild-type rare-cutting endonuclease (e.g., Cas9 from S. pyogenes, supra) over 10 contiguous amino acids in a HNH nuclease domain of the rare-cutting endonuclease. The rare-cutting endonuclease can comprise at least: 70, 75, 80, 85, 90, 95, 97, 99, or 100% identity to a wild-type rare-cutting endonuclease (e.g., Cas9 from S. pyogenes, supra) over 10 contiguous amino acids in a RuvC nuclease domain of the rare-cutting endonuclease. The rare-cutting endonuclease can comprise at most: 70, 75, 80, 85, 90, 95, 97, 99, or 100% identity to a wild-type rare-cutting endonuclease (e.g., Cas9 from S. pyogenes, supra) over 10 contiguous amino acids in a RuvC nuclease domain of the rare-cutting endonuclease.

A modified form of the rare-cutting endonuclease can comprise a mutation such that it can induce a single-strand break (SSB) on a target nucleic acid (e.g., by cutting only one of the sugar-phosphate backbones of a double-strand target nucleic acid). In some aspects, the mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type rare-cutting endonuclease (e.g., Cas9 from S. pyogenes, supra). In some aspects, the mutation can result in one or more of the plurality of nucleic acid-cleaving domains retaining the ability to cleave the complementary strand of the target nucleic acid, but reducing its ability to cleave the non-complementary strand of the target nucleic acid. The mutation can result in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp10, His840, Asn854 and Asn856, are mutated to inactivate one or more of the plurality of nucleic acid-cleaving domains (e.g., nuclease domains). The residues to be mutated can correspond to residues Asp10, His840, Asn854 and Asn856 in the wild-type exemplary S. pyogenes Cas9 polypeptide (e.g., as determined by sequence and/or structural alignment). Non-limiting examples of mutations include DlOA, H840A, N854A or N856A. One skilled in the art will recognize that mutations other than alanine substitutions can be suitable.

Nickase variants of RNA-guided endonucleases, for example Cas9, can be used to increase the specificity of CRISPR-mediated genome editing. Wild type Cas9 is typically guided by a single guide RNA designed to hybridize with a specified ⁻20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HR versus NHEJ. HR can be used to introduce selected changes into target sites in the genome through the use of specific donor sequences that effectively mediate the desired changes.

The one or more rare-cutting endonucleases, e.g. DNA endonucleases, can comprise two nickases that together effect one double-strand break at a specific locus in the genome, or four nickases that together effect or cause two double-strand breaks at specific loci in the genome. Alternatively, one rare-cutting endonuclease, e.g. DNA endonuclease, can effect or cause one double-strand break at a specific locus in the genome.

Non-limiting examples of Ca.s9 orthologs from other bacterial strains include but are Foot limited to, Cas proteins identified in Acaryochloris marina MBIC11017; Acetohalobium arabaticum DSM 5501; Acidithiobaciihrs caldus: Acidithiobacillus ferrooxiclans ATCC 23270; Alicyclobacillus acidocaldarius LAA1; Alicyclobacillus acidocaldarius subsp. acidocaldarius DSM 446; Allochromatium vinosum DSM 180,. Ammonex degensii KC4; Anabaena variabilis ATCC 29413; Arthrospira maxima CS-328; Arthrospira platensis str. Paraca; Arihrospira sp. PCC 8005; Bacillus pseudomycoides DSM 12442; Bacillus selenitireducens MIS10; Burkholderiales bacterium 1_1_47; Caldicelulosiruptor becscii DSM 6725; Candidatus Desulforudis audaxviator MP104C; Caldicellulosiruptor hydrothermalis_108; Clostridium phage c-st; Clostridium botuhnum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacternim sibiricum 255-15; Finegoldia magna ATCC 29328; Ktedonohacter raceurifer DSM 44963; Lactobacillus delbrueckii subsp. bulgaricus PB2003/044-T3-4; Lactobacillus salivarius ATCC 11741; Listeria innocua; Lyngbva sp. PCC 8106; Marinobacter sp. ELB 17; Methanohalobium evestigatum Z-7303; Microcystis phage Ma-LMM01; Microcystis aeruginosa NIES-843; Microscilla marina ATCC 23134; Microcoleus chthonoplastes PCC 7420; Neisseria meningitidis; Nitrosococcus halophihis N_{c}4; Nocardiopsis dassonvillei subsp. dassonvillei DSM 43111; Nodularia spumigena CCY9414; Nostoc sp. PCC 7120; Oscillatoria sp. PCC 6506; Pelotomaculum thermopropionicum_SI; Petrotoga mobilis SJ95; Polaromonas naphthalenivorans C12; Polaromonas sp. JS666; Pseudoalteromonas haopauktis TAC 125 ; Streptomyces pristitiaespiralis ATCC 25486; Streptomyces pristinaespirahs ATCC 25486; Streptococcus thermophilus; Streptomyces viridochromogeues DSM 40736; Streptosporarigium roseum DSM 43021; Synechoroccus sp. PCC 7335; and Thermosipho africamus TCF52B (Chylinski et al., RNA Biol., 2013; 10(5): 726-737).

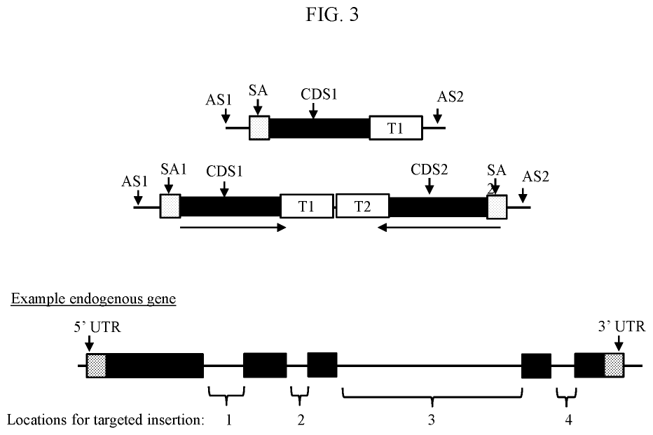

In some embodiments, this document features a transgene with a corrective coding sequence resistant to silencing operably linked to a splice acceptor and terminator and flanked by additional sequences. This document also features a transgene with a bidirectional corrective coding sequence resistant to silencing operably linked to a splice acceptor and terminator and flanked by additional sequences (FIG. 3). The transgenes can be integrated in an endogenous gene within an intron or at intron-exon junctions.

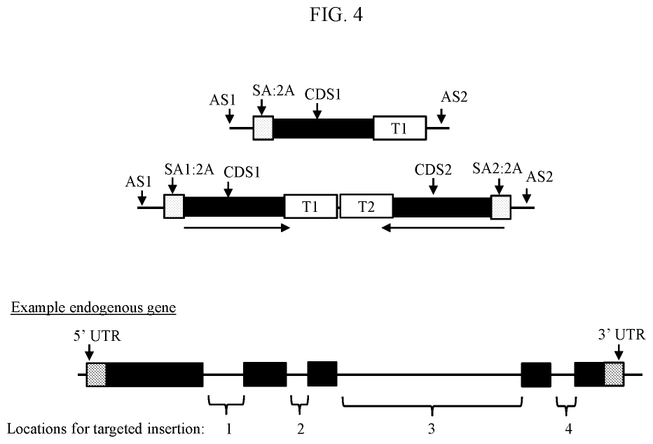

In some embodiments, this document features a transgene with a corrective coding sequence resistant to silencing operably linked to a splice acceptor, 2A sequence, and terminator and flanked by additional sequences. This document also features a transgene with a bidirectional corrective coding sequence resistant to silencing operably linked to a splice acceptor, 2A sequence, and terminator and flanked by additional sequences (FIG. 4). The transgenes can be integrated in an endogenous gene within an intron or at intron-exon junctions.

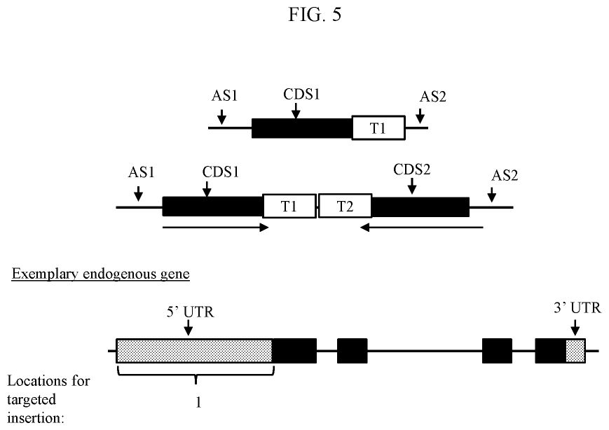

In some embodiments, this document features a transgene with a corrective coding sequence resistant to silencing operably linked to a terminator and flanked with additional sequences. This document also features a transgene with a bidirectional corrective coding sequence resistant to silencing operably linked to terminators and flanked by additional sequences (FIG. 5). The transgenes can be integrated within a the 5′ UTR of an endogenous gene.

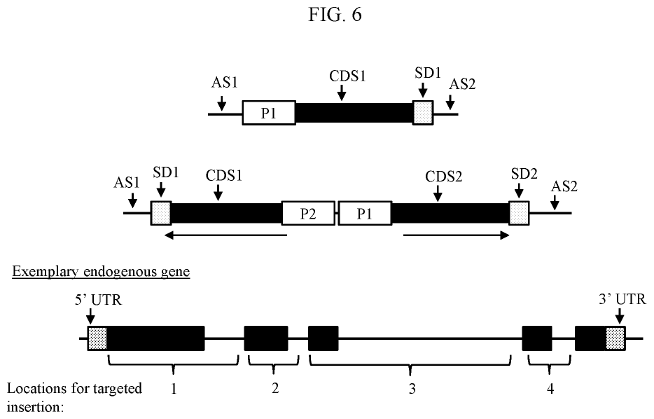

In some embodiments, this document features a transgene with a promoter operably linked to a corrective coding sequence resistant to silencing operably linked to splice donor and flanked with additional sequences. This document also features a transgene with a bidirectional promoter operably linked to a corrective coding sequence resistant to silencing operably linked to splice donor and flanked with additional sequences (FIG. 6). The transgenes can be integrated within the exons or introns of an endogenous gene.

In some embodiments, this document features a transgene with a splice acceptor operably linked to a 2A sequence operably linked to a corrective coding sequence resistant to silencing operably linked to splice donor and flanked with additional sequences (FIG. 7). This transgene can be integrated within the introns of an endogenous gene.

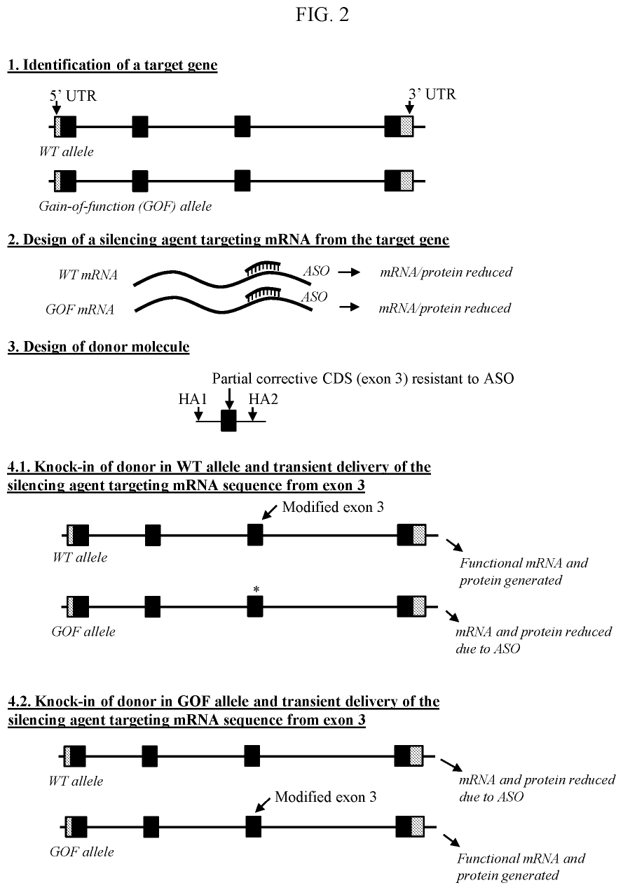

In an embodiment, this document features a method for modifying sequence within an endogenous gene by administering a donor molecule that i) corrects mutations within a target site and ii) introduces sequence changes to prevent silencing by a silencing agent. The donor molecule can comprise two homology arms and a cargo sequence (FIG. 2). After administrating the donor molecule, the cells can be administered the silencing agent.

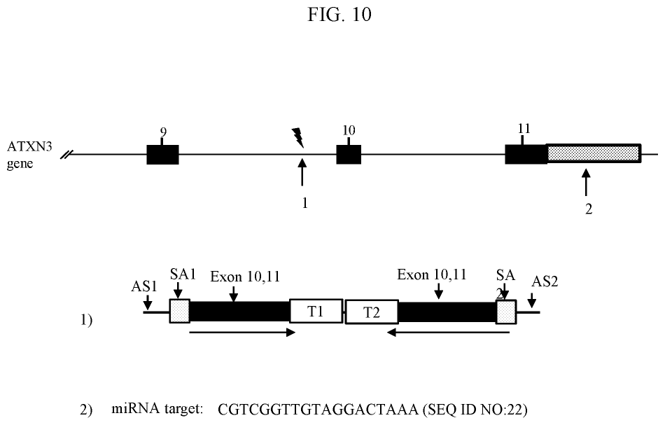

In an embodiment, this document features a method for modifying the ATXN3 gene, the method comprising administering a transgene with a corrective coding sequence resistant to silencing operably linked to a splice acceptor and terminator and flanked by additional sequences. The coding sequence can comprise sequence encoding the peptide produced by exons 10-11 of the endogenous ATXN3 gene. This document also features a transgene with a bidirectional corrective coding sequence resistant to silencing operably linked to a splice acceptor and terminator and flanked by additional sequences (FIG. 10). The transgenes can be integrated in intron 9 of the endogenous ATXN3 gene.

The methods and compositions described in this document can be used in a circumstance where it is desired to treat cells with gain-of-function mutations. For example, patients with SCA3 have expanded trinucleotide repeats in exon 10. Patients with SCA3 may benefit from replacement of exon 10 and 11, followed by delivery of a silencing agent that reduces the expression of any remaining unmodified genes with the gain-of-function mutation. Additional benefits of this approach include the ability to choose a target site for silencing that is not centered around the gain-of-function mutation site. This benefit enables the design of the effective silencing constructs (e.g., low off-targeting and highly effective on-targeting). Further the methods can be particularly useful in gain-of-function disorders with genes that produce multiple isoforms, including SCA3.

The methods provided herein will be further described in the following examples, which do not limit the scope of the invention described in the claims.

SEQUENCES

SEQ ID NO: 1 <PBA1135-D1; DNA; ARTIFICIAL SEQUENCE>
ATTTCATTTATCAGGTGTTCAGTGAATGCTTACTATGTAACAGCACAGTTA
TCAGCACTGGGGAAATAGATGAGTAAGATAAGATTTGCACTTTCATTAGCTTACA
TGCCATAAAGAGGGAAATAAAGAGAACACCAGATGATGATAAGTTTATGCTGAG
AATTAAAATGAAGTGATGAAATAATGGGAATGTCAGGTGGCTACTTTTGGTGGG
ATGGTCAGGAAAGGCATCTCTGGGGAGATAAATTTTAAGCTCAGACCTGAGTGA
AAAGAATGAGCCAGCCATGGAAACATTATGTTAACTCACATGGTAGTTTGAAATG
CTTTATCTGATCAAAGGTACTTATTTTTGGTGACTTTCAACAATATTAAGGGTCTA
TAAACCAACACTCATTTGCATAAGAATAACTACCAGTGAATCTTTTTGTATGATA
GGTTTTTTGTTTGTTGTTTTTTTGAGACAGAGTCTCGCTCTGTCGCCCAGGCTGGA
GTGCAGTGGCGCGATCTTGGCTCACTGCAACCTCTACCTCCCCGGTTCAAGTGAT
TCTCCTGCCTCAGCCTCCCAAAGTAGCTGGGATTACAGGTGCCTGCCACCACGCC
TGGCTAATTTTTGTATTTTTAGTAGAGATGGGGTTTCACCGTGTTGTCCAGGCTCG
TGTCAAACTTCTGACCTCAAGCCATCCACCCGCCTCGGCCTCCCAAAGTGCTGGG
ATTACAGGTGTGAGCCACCACTCCTGGCCATGATAGGTTATTTTGTGATGAAAAT
ACCTACCTCTTAATTTGTCTGATAAATTTAAATTTTATGTCTAGATTTCCTAAGAT
CAGCACTTCCATATTTTAAAGTAATCTGTATCAGACTAACTGCTCTTGCATTCTTT
TAATACCAGTGACTACTTTGATTCGTGAAACAATGTATTTTCCTTATGAATAGTTT
TTCTCATGGTGTATTTATTCTTTTAAGTTTTGTTTTTTAAATATACTTCACTTTTGA
ATGTTTCAGACAGCAGCAAAAGCAGCAACAGCAGCAGCAGCAGCAGCAGCAGG
GGGACCTATCAGGACAGAGTTCACATCCATGTGAAAGGCCAGCCACCAGTTCAG
GAGCACTTGGGAGTGATCTAGGTGATGCTATGAGTGAAGAAGACATGCTTCAGG
CAGCTGTGACCATGTCTTTAGAAACTGTCAGAAATGATTTGAAAACAGAAGGAA
AAAAATAAAACTTGTTTATTGCAGCTTATAATGGTTACAAATAAAGCAATAGCAT
CACAAATTTCACAAATAAAGCATTTTTTTCACTGCATTCTAGTTGTGGTTTGTCCA
AACTCATCAATGTATCTTATCATGTCTGGATCTCCCCAGCATGCCTGCTATTCTCT
TCCCAATCCTCCCCCTTGCTGTCCTGCCCCACCCCACCCCCCAGAATAGAATGAC
ACCTACTCAGACAATGCGATGCAATTTCCTCATTTTATTAGGAAAGGACAGTGGG
AGTGGCACCTTCCAGGGTCAAGGAAGGCACGGGGGAGGGGCAAACAACAGATG
GCTGGCAACTAGAAGGCACAGCTACTTCTTGCCCTCGGTCTTCAGGTCGTTGCGC
ACGGTCTCCAGGCTCATGGTCACGGCGGCCTGCAGCATGTCCTCCTCGCTCATGG
CGTCGCCCAGGTCGCTGCCCAGGGCGCCGCTGCTGGTGGCGGGGCGCTCGCAGG
GGTGGCTGCTCTGGCCGCTCAGGTCGCCCTGCTGCTGCTGCTGCTGCTGCTGCTGC
TGCTTCTGCTGCTGTCTGTAAATGAATGAGAAAACCGGTTTAGAAAGTGCACAGC
TGTCAGGGAAGTCAACACTTCAGTGAGCATGTGACCATGTGGAGTCAGCTTCCTG
TTTCGTGCTGCAATCGTAAGGCCTGCTCACCATTCATCATGTTCGCTACCTTCACA
CTTTATCTGACATACGAGCTCCATGTGATTTTTGCTTTACATTATTCTTCATTCCCT
CTTTAATCATATTAAGAATCTTAAGTAAATTTGTAATCTACTAAATTTCCCTGGAT
TAAGGAGCAGTTACCAAAAGAAAAAAAAAAAAAAAAGCTAGATGTGGTGGCTC
ACATCTGTAATCCCAGCACTTTGGGAAACCAAGGCAGGAGAGGATTGCTAGAAC
ATTTAATGAATACTTTAACATAATAATTTAAACTTCACAGTAATTTGTACAGTCTC
CAAAAATTCCTTAGACATCATGGATATTTTTCTTTTTTTGAGATGGAGTCTTGCTC
TTTTAAGCTCAGACCTGAGTGAAAAGAATTTGAGACAGAGTCTCGCTCTGTCGCC
TTTCCTAAGATCAGCACTTCCATATTTGGTGACTTTCAACAATATTAAGGGTCTAT
AAACCAACACTCATTTGCATAAGAAT
SEQ ID NO: 2 <PBA113 5-C1 GRNA TARGET; DNA; ARTIFICIAL SEQUENCE>
AATATGGAAGTGCTGATCTT
SEQ ID NO: 3 <PBA1135 LHA; DNA; ARTIFICIAL SEQUENCE>
ATTTCATTTATCAGGTGTTCAGTGAATGCTTACTATGTAACAGCACAGTTA
TCAGCACTGGGGAAATAGATGAGTAAGATAAGATTTGCACTTTCATTAGCTTACA
TGCCATAAAGAGGGAAATAAAGAGAACACCAGATGATGATAAGTTTATGCTGAG
AATTAAAATGAAGTGATGAAATAATGGGAATGTCAGGTGGCTACTTTTGGTGGG
ATGGTCAGGAAAGGCATCTCTGGGGAGATAAATTTTAAGCTCAGACCTGAGTGA
AAAGAATGAGCCAGCCATGGAAACATTATGTTAACTCACATGGTAGTTTGAAATG
CTTTATCTGATCAAAGGTACTTATTTTTGGTGACTTTCAACAATATTAAGGGTCTA
TAAACCAACACTCATTTGCATAAGAATAACTACCAGTGAATCTTTTTGTATGATA
GGTTTTTTGTTTGTTGTTTTTTTGAGACAGAGTCTCGCTCTGTCGCCCAGGCTGGA
GTGCAGTGGCGCGATCTTGGCTCACTGCAACCTCTACCTCCCCGGTTCAAGTGAT
TCTCCTGCCTCAGCCTCCCAAAGTAGCTGGGATTACAGGTGCCTGCCACCACGCC
TGGCTAATTTTTGTATTTTTAGTAGAGATGGGGTTTCACCGTGTTGTCCAGGCTCG
TGTCAAACTTCTGACCTCAAGCCATCCACCCGCCTCGGCCTCCCAAAGTGCTGGG
ATTACAGGTGTGAGCCACCACTCCTGGCCATGATAGGTTATTTTGTGATGAAAAT
ACCTACCTCTTAATTTGTCTGATAAATTTAAATTTTATGTCTAGATTTCCTAAGAT
CAGCACTTCCATATTTTAAAGTAATCTGTATCAGACTAACTGCTCTTGCATTCTTT
TAATACCAGTGACTACTTTGATTCGTGAAACAATGTATTTTCCTTATGAATAGTTT
TTCTCATGGTGTATTTATTCTTTTAAGTTTTGTTTTTTAAATATACTTCACTTTTGA
ATGTTTCAG
SEQ ID NO: 4 <PBA1135 PARTIAL CODING SEQUENCE; DNA; ARTIFICIAL
SEQUENCE>
ACAGCAGCAAAAGCAGCAACAGCAGCAGCAGCAGCAGCAGCAGGGGGA
CCTATCAGGACAGAGTTCACATCCATGTGAAAGGCCAGCCACCAGTTCAGGAGC
ACTTGGGAGTGATCTAGGTGATGCTATGAGTGAAGAAGACATGCTTCAGGCAGCT
GTGACCATGTCTTTAGAAACTGTCAGAAATGATTTGAAAACAGAAGGAAAAAAA
TAA
SEQ ID NO: 5<PBA1135 TERMINATOR; DNA; ARTIFICIAL SEQUENCE>
AACTTGTTTATTGCAGCTTATAATGGTTACAAATAAAGCAATAGCATCAC
AAATTTCACAAATAAAGCATTTTTTTCACTGCATTCTAGTTGTGGTTTGTCCAAAC
TCATCAATGTATCTTATCATGTCTGGATC
SEQ ID NO: 6 <PBA1135 TERMINATOR; DNA; ARTIFICIAL SEQUENCE>
TCCCCAGCATGCCTGCTATTCTCTTCCCAATCCTCCCCCTTGCTGTCCTGCC
CCACCCCACCCCCCAGAATAGAATGACACCTACTCAGACAATGCGATGCAATTTC
CTCATTTTATTAGGAAAGGACAGTGGGAGTGGCACCTTCCAGGGTCAAGGAAGG
CACGGGGGAGGGGCAAACAACAGATGGCTGGCAACTAGAAGGCACAG
SEQ ID NO: 7 <PBA1135 PARTIAL CODING SEQUENCE; DNA; ARTIFICIAL
SEQUENCE>
CTACTTCTTGCCCTCGGTCTTCAGGTCGTTGCGCACGGTCTCCAGGCTCAT
GGTCACGGCGGCCTGCAGCATGTCCTCCTCGCTCATGGCGTCGCCCAGGTCGCTG
CCCAGGGCGCCGCTGCTGGTGGCGGGGCGCTCGCAGGGGTGGCTGCTCTGGCCG
CTCAGGTCGCCCTGCTGCTGCTGCTGCTGCTGCTGCTGCTGCTTCTGCTGCTGT
SEQ ID NO: 8 <PBA1135 SPLICE ACCEPTOR; DNA; ARTIFICIAL SEQUENCE>
CTGTAAATGAATGAGAAAACCGGTTTAGAAAGTGCACAGCTGTCAGGGA
AGTCAACACTTCAGTGAGCATGTGACCATGTGGAGTCAGCTTCCTGTTTCGTGCT
GCAATC
SEQ ID NO: 9 <PBA1135 RHA; DNA; ARTIFICIAL SEQUENCE>
GTAAGGCCTGCTCACCATTCATCATGTTCGCTACCTTCACACTTTATCTGA
CATACGAGCTCCATGTGATTTTTGCTTTACATTATTCTTCATTCCCTCTTTAATCAT
ATTAAGAATCTTAAGTAAATTTGTAATCTACTAAATTTCCCTGGATTAAGGAGCA
GTTACCAAAAGAAAAAAAAAAAAAAAAGCTAGATGTGGTGGCTCACATCTGTAA
TCCCAGCACTTTGGGAAACCAAGGCAGGAGAGGATTGCTAGAACATTTAATGAA
TACTTTAACATAATAATTTAAACTTCACAGTAATTTGTACAGTCTCCAAAAATTCC
TTAGACATCATGGATATTTTTCTTTTTTTGAGATGGAGTCTTGCTCT
SEQ ID NO: 10 <PBA1135 NUCLEASE TARGET SITE; DNA; ARTIFICIAL
SEQUENCE>
TTTAAGCTCAGACCTGAGTGAAAAGAATTTGAGACAGAGTCTCGCTCTGT
CGCCTTTCCTAAGATCAGCACTTCCATATTTGGTGACTTTCAACAATATTAAGGGT
CTATAAACCAACACTCATTTGCATAAGAAT
SEQ ID NO: 11 <PBA1136-D1; DNA; ARTIFICIAL SEQUENCE>
TTTAAGCTCAGACCTGAGTGAAAAGAATTTGAGACAGAGTCTCGCTCTGT
CGCCTTTCCTAAGATCAGCACTTCCATATTTTAAAGTAATCTGTATCAGACTAACT
GCTCTTGCATTCTTTTAATACCAGTGACTACTTTGATTCGTGAAACAATGTATTTT
CCTTATGAATAGTTTTTCTCATGGTGTATTTATTCTTTTAAGTTTTGTTTTTTAAAT
ATACTTCACTTTTGAATGTTTCAGACAGCAGCAAAAGCAGCAACAGCAGCAGCA
GCAGCAGCAGCAGGGGGACCTATCAGGACAGAGTTCACATCCATGTGAAAGGCC
AGCCACCAGTTCAGGAGCACTTGGGAGTGATCTAGGTGATGCTATGAGTGAAGA
AGACATGCTTCAGGCAGCTGTGACCATGTCTTTAGAAACTGTCAGAAATGATTTG
AAAACAGAAGGAAAAAAATAAAACTTGTTTATTGCAGCTTATAATGGTTACAAA
TAAAGCAATAGCATCACAAATTTCACAAATAAAGCATTTTTTTCACTGCATTCTA
GTTGTGGTTTGTCCAAACTCATCAATGTATCTTATCATGTCTGGATCTCCCCAGCA
TGCCTGCTATTCTCTTCCCAATCCTCCCCCTTGCTGTCCTGCCCCACCCCACCCCCC
AGAATAGAATGACACCTACTCAGACAATGCGATGCAATTTCCTCATTTTATTAGG
AAAGGACAGTGGGAGTGGCACCTTCCAGGGTCAAGGAAGGCACGGGGGAGGGG
CAAACAACAGATGGCTGGCAACTAGAAGGCACAGCTACTTCTTGCCCTCGGTCTT
CAGGTCGTTGCGCACGGTCTCCAGGCTCATGGTCACGGCGGCCTGCAGCATGTCC
TCCTCGCTCATGGCGTCGCCCAGGTCGCTGCCCAGGGCGCCGCTGCTGGTGGCGG
GGCGCTCGCAGGGGTGGCTGCTCTGGCCGCTCAGGTCGCCCTGCTGCTGCTGCTG
CTGCTGCTGCTGCTGCTTCTGCTGCTGTCTGTAAATGAATGAGAAAACCGGTTTA
GAAAGTGCACAGCTGTCAGGGAAGTCAACACTTCAGTGAGCATGTGACCATGTG
GAGTCAGCTTCCTGTTTCGTGCTGCAATCTTTAAGCTCAGACCTGAGTGAAAAGA
ATTTGAGACAGAGTCTCGCTCTGTCGCCTTTCCTAAGATCAGCACTTCCATATTT
SEQ ID NO: 12 <PBA1137-D1; DNA; ARTIFICIAL SEQUENCE>
ATTTCATTTATCAGGTGTTCAGTGAATGCTTACTATGTAACAGCACAGTTA
TCAGCACTGGGGAAATAGATGAGTAAGATAAGATTTGCACTTTCATTAGCTTACA
TGCCATAAAGAGGGAAATAAAGAGAACACCAGATGATGATAAGTTTATGCTGAG
AATTAAAATGAAGTGATGAAATAATGGGAATGTCAGGTGGCTACTTTTGGTGGG
ATGGTCAGGAAAGGCATCTCTGGGGAGATAAATTTTAAGCTCAGACCTGAGTGA
AAAGAATGAGCCAGCCATGGAAACATTATGTTAACTCACATGGTAGTTTGAAATG
CTTTATCTGATCAAAGGTACTTATTTTTGGTGACTTTCAACAATATTAAGGGTCTA
TAAACCAACACTCATTTGCATAAGAATAACTACCAGTGAATCTTTTTGTATGATA
GGTTTTTTGTTTGTTGTTTTTTTGAGACAGAGTCTCGCTCTGTCGCCCAGGCTGGA
GTGCAGTGGCGCGATCTTGGCTCACTGCAACCTCTACCTCCCCGGTTCAAGTGAT
TCTCCTGCCTCAGCCTCCCAAAGTAGCTGGGATTACAGGTGCCTGCCACCACGCC
TGGCTAATTTTTGTATTTTTAGTAGAGATGGGGTTTCACCGTGTTGTCCAGGCTCG
TGTCAAACTTCTGACCTCAAGCCATCCACCCGCCTCGGCCTCCCAAAGTGCTGGG
ATTACAGGTGTGAGCCACCACTCCTGGCCATGATAGGTTATTTTGTGATGAAAAT
ACCTACCTCTTAATTTGTCTGATAAATTTAAATTTTATGTCTAGAAATCCTAAGAT
CAGCACTTCCATATTTTAAAGTAATCTGTATCAGACTAACTGCTCTTGCATTCTTT
TAATACCAGTGACTACTTTGATTCGTGAAACAATGTATTTTCCTTATGAATAGTTT
TTCTCATGGTGTATTTATTCTTTTAAGTTTTGTTTTTTAAATATACTTCACTTTTGA
ATGTTTCAGACAGCAGCAAAAGCAGCAACAGCAGCAGCAGCAGCAGCAGCAGG
GGGACCTATCAGGACAGAGTTCACATCCATGTGAAAGGCCAGCCACCAGTTCAG
GAGCACTTGGGAGTGATCTAGGTGATGCTATGAGTGAAGAAGACATGCTTCAGG
CAGCTGTGACCATGTCTTTAGAAACTGTCAGAAATGATTTGAAAACAGAAGGAA
AAAAATAAAACTTGTTTATTGCAGCTTATAATGGTTACAAATAAAGCAATAGCAT
CACAAATTTCACAAATAAAGCATTTTTTTCACTGCATTCTAGTTGTGGTTTGTCCA
AACTCATCAATGTATCTTATCATGTCTGGATCGTAAGGCCTGCTCACCATTCATCA
TGTTCGCTACCTTCACACTTTATCTGACATACGAGCTCCATGTGATTTTTGCTTTA
CATTATTCTTCATTCCCTCTTTAATCATATTAAGAATCTTAAGTAAATTTGTAATCT
ACTAAATTTCCCTGGATTAAGGAGCAGTTACCAAAAGAAAAAAAAAAAAAAAAG
CTAGATGTGGTGGCTCACATCTGTAATCCCAGCACTTTGGGAAACCAAGGCAGGA
GAGGATTGCTAGAACATTTAATGAATACTTTAACATAATAATTTAAACTTCACAG
TAATTTGTACAGTCTCCAAAAATTCCTTAGACATCATGGATATTTTTCTTTTTTTG
AGATGGAGTCTTGCTCT
SEQ ID NO: 13<PRIMER; DNA; ARTIFICIAL SEQUENCE>
CAAAGGTGCCCTTGAGGTT
SEQ ID NO: 14<PRIMER; DNA; ARTIFICIAL SEQUENCE>
AGGAGAAGTCTGCCGTTACT
SEQ ID NO: 15<PRIMER; DNA; ARTIFICIAL SEQUENCE>
GGACAAACCACAACTAGAATGC
SEQ ID NO: 16<PRIMER; DNA; ARTIFICIAL SEQUENCE>
TAGGAAAGGACAGTGGGAGT
SEQ ID NO: 17<PRIMER; DNA; ARTIFICIAL SEQUENCE>
CCATTATGTCTCAGTTGTTCAGTG
SEQ ID NO: 18<PRIMER; DNA; ARTIFICIAL SEQUENCE>
CCAGACCATCTCAGACACC
SEQ ID NO: 19<PRIMER; DNA; ARTIFICIAL SEQUENCE>
GGCTGGGCTTCCACTTAC
SEQ ID NO: 20<PRIMER; DNA; ARTIFICIAL SEQUENCE>
GTGGTTTGTCCAAACTCATCAA
SEQ ID NO: 21<PRIMER; DNA; ARTIFICIAL SEQUENCE>
AGTAACTCTGCACTTCCCATTG
SEQ ID NO: 22<ATXN3 SIRNA; RNA; ARTIFICIAL SEQUENCE>
CGUCGGUUGUAGGACUAAA
SEQ ID NO: 23<SOD1 SHRNA; RNA; ARTIFICIAL SEQUENCE>
GGCCUGCAUGGAUUCCAUG
SEQ ID NO: 24<gRNA; DNA; Artificial Sequence>
GTGTGTTACTAATTTTATAAATGGAGT
SEQ ID NO: 25<gRNA; DNA; Artificial Sequence>
TAATTAAAAAAAAATGCTAGGCAGAAT
SEQ ID NO: 26 <pBA1801; DNA; Artificial Sequence>
TTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGC
GAGCGAGCGCGCAGAGAGGGAGTGGCCAACTCCATCACTAGGGGTTCCTT
AGGTCAGTGAAGAGAAGAACAAAAAGCAGCATATTACAGTTAGTTGTCTT
CATCAATCTTTAAATATGTTGTGTGGTTTTTCTCTCCCTGTTTCCACAGTTG
AGGACCCCCAGGGCGACGCCGCCCAGAAGACCGACACCAGCCACCACGA
CCAGGACCACCCCACCTTCAACAAGATCACCCCCAACCTGGCCGAGTTCG
CCTTCAGCCTGTACAGGCAGCTGGCCCACCAGAGCAACAGCACCAACATC
TTCTTCAGCCCCGTGAGCATCGCCACCGCCTTCGCCATGCTGAGCCTGGGC
ACCAAGGCCGACACCCACGACGAGATCCTGGAGGGCCTGAACTTCAACCT
GACCGAGATCCCCGAGGCCCAGATCCACGAGGGCTTCCAGGAGCTGCTGA
GGACCCTGAACCAGCCCGACAGCCAGCTGCAGCTGACCACCGGCAACGG
CCTGTTCCTGAGCGAGGGCCTGAAGCTGGTGGACAAGTTCCTGGAGGACG
TGAAGAAGCTGTACCACAGCGAGGCCTTCACCGTGAACTTCGGCGACACC
GAGGAGGCCAAGAAGCAGATCAACGACTACGTGGAGAAGGGCACCCAGG
GCAAGATCGTGGACCTGGTGAAGGAGCTGGACAGGGACACCGTGTTCGCC
CTGGTGAACTACATCTTCTTCAAGGGCAAGTGGGAGAGGCCCTTCGAGGT
GAAGGACACCGAGGAGGAGGACTTCCACGTGGACCAGGTGACCACCGTG
AAGGTGCCCATGATGAAACGCCTCGGAATGTTCAACATCCAGCACTGCAA
GAAGCTGAGCAGCTGGGTGCTGCTGATGAAGTACCTGGGCAACGCCACCG
CCATCTTCTTCCTGCCCGACGAGGGCAAGCTGCAGCACCTGGAGAACGAG
CTGACCCACGACATCATCACCAAGTTCCTGGAGAACGAGGACAGGAGGA
GCGCCAGCCTGCACCTGCCCAAGCTGAGCATCACCGGCACCTACGACCTG
AAGAGCGTGCTGGGCCAGCTGGGCATCACCAAGGTGTTCAGCAACGGCGC
CGACCTGAGCGGCGTGACCGAGGAGGCCCCCCTGAAGCTGAGCAAGGCC
GTGCACAAGGCCGTGCTGACCATCGACGAGAAGGGCACCGAGGCCGCCG
GCGCCATGTTCCTGGAGGCCATCCCCATGAGCATCCCCCCCGAGGTGAAG
TTCAACAAGCCCTTCGTGTTCCTGATGATCGAGCAGAACACCAAGAGCCC
CCTGTTCATGGGCAAGGTGGTGAACCCCACCCAGAAGTAACAGACATGAT
AAGATACATTGATGAGTTTGGACAAACCACAACTAGAATGCAGTGAAAA
AAATGCTTTATTTGTGAAATTTGTGATGCTATTGCTTTATTTGTAACCATTA
TAAGCTGCAATAAACAAGTTAACAACAACAATTGCATTCATTTTATGTTTC
AGGTTCAGGGGGAGGTGTGGGAGGTTTTTTGGGGATACCCCCTAGAGCCC
CAGCTGGTTCTTTCCGCCTCAGAAGCCATAGAGCCCACCGCATCCCCAGC
ATGCCTGCTATTGTCTTCCCAATCCTCCCCCTTGCTGTCCTGCCCCACCCCA
CCCCCCAGAATAGAATGACACCTACTCAGACAATGCGATGCAATTTCCTC
ATTTTATTAGGAAAGGACAGTGGGAGTGGCACCTTCCAGGGTCAAGGAAG
GCACGGGGGAGGGGCAAACAACAGATGGCTGGCAACTAGAAGGCACAGT
CGAGGttaTTTTTGGGTGGGATTCACCACTTTTCCCATGAAGAGGGGAGACT
TGGTATTTTGTTCAATCATTAAGAAGACAAAGGGTTTGTTGAACTTGACCT
CGGGGGGGATAGACATGGGTATGGCCTCTAAAAACATGGCCCCAGCAGCT
TCAGTCCCTTTCTCGTCGATGGTCAGCACAGCCTTATGCACGGCCTTGGAG
AGCTTCAGGGGTGCCTCCTCTGTGACCCCGGAGAGGTCAGCCCCATTGCT
GAAGACCTTAGTGATGCCCAGTTGACCCAGGACGCTCTTCAGATCATAGG
TTCCAGTAATGGACAGTTTGGGTAAATGTAAGCTGGCAGACCTTCTGTCTT
CATTTTCCAGGAACTTGGTGATGATATCGTGGGTGAGTTCATTTTCCAGGT
GCTGTAGTTTCCCCTCATCAGGCAGGAAGAAGATGGCGGTGGCATTGCCC
AGGTATTTCATCAGCAGCACCCAGCTGGACAGCTTCTTACAGTGCTGGAT
GTTGAACATTCCGAGGCGTTTCATCATAGGCACCTTCACGGTGGTCACCTG
GTCCACGTGGAAGTCCTCTTCCTCGGTGTCCTTGACTTCAAAGGGTCTCTC
CCATTTGCCTTTAAAGAAGATGTAATTCACCAGAGCAAAAACTGTGTCTCT
GTCAAGCTCCTTGACCAAATCCACAATTTTCCCTTGAGTACCCTTCTCCAC
GTAATCGTTGATCTGTTTCTTGGCCTCTTCGGTGTCCCCGAAGTTGACAGT
GAAGGCTTCTGAGTGGTACAACTTTTTAACATCCTCCAAAAACTTATCCAC
TAGCTTCAGGCCCTCGCTGAGGAACAGGCCATTGCCGGTGGTCAGCTGGA
GCTGGCTGTCTGGCTGGTTGAGGGTACGGAGGAGTTCCTGGAAGCCTTCA
TGGATCTGAGCCTCCGGAATCTCCGTGAGGTTGAAATTCAGGCCCTCCAG
GATTTCATCGTGAGTGTCAGCCTTGGTCCCCAGGGAGAGCATTGCAAAGG
CTGTAGCGATGCTCACTGGGGAGAAGAAGATATTGGTGCTGTTGGACTGG
TGTGCCAGCTGGCGGTATAGGCTGAAGGCGAACTCAGCCAGGTTGGGGGT
GATCTTGTTGAAGGTTGGGTGATCCTGATCATGGTGGGATGTATCTGTCTT
CTGGGCAGCATCTCCCTGGGGATCCTCAACTGAAATGTAAAAGAATAATT
CTTTAGTTTTAGCAAAAAAGAAAACATCATGAAAATTTTACATCTCTTAAG
AAAGTCTTTGTTTTTAATCCAAATAATCAGGAACCCCTAGTGATGGAGTTG
GCCACTCCCTCTCTGCGCGCTCGCTCGCTCACTGAGGCCGCCCGGGCAAA
GCCCGGGCGTCGGGCGACCTTTGGTCGCCCGGCCTCAGTGAGCGAGCGAG
CGCGCAGAGAGGGAGTGGCCAA
SEQ ID NO: 27 <SERPINA1 shRNA; RNA; Artificial Sequence>
GAUGAAGCGUUUAGGCAUGUU
SEQ ID NO: 28 <SERPINA1 shRNA; RNA; Artificial Sequence>
GAACUCACCCACGAUAUCAUU
SEQ ID NO: 29 <SERPINA1 shRNA; RNA; Artificial Sequence>
CCUAUGAUCUGAAGAGCGUUU
SEQ ID NO: 30 <SERPINA1 shRNA; RNA; Artificial Sequence>
UGAUAUCGUGGGUGAGUUCUU
SEQ ID NO: 31 <SERPINA1 shRNA; RNA; Artificial Sequence>
CAUGCCUAAACGCUUCAUCUU
SEQ ID NO: 32 <SERPINA1 shRNA; RNA; Artificial Sequence>
ACGCUCUUCAGAUCAUAGGUU
SEQ ID NO: 33 <gRNA; DNA; Artificial Sequence>
TTCAACTGTATCCAACGTAATTTGAGT
SEQ ID NO: 34 <gRNA; DNA; Artificial Sequence>
CTCAAATTACGTTGGATACAGTTGAAT
SEQ ID NO: 35 <gRNA; DNA; Artificial Sequence>
ACATGACAGAAACACTAAATCTTGAGT
SEQ ID NO: 36 <gRNA; DNA; Artificial Sequence>
GATCGGGAACTGGCATCTTCAGGGAGT
SEQ ID NO: 37 <gRNA; DNA; Artificial Sequence>
GGCAGAATGACTCAAATTACGTTGGAT
SEQ ID NO: 38 <gRNA; DNA; Artificial Sequence>
GTATCTTTGATGACAATAATGGGGGAT
SEQ ID NO: 39 <gRNA; DNA; Artificial Sequence>
CAGAAACACTAAATCTTGAGTTTGAAT
SEQ ID NO: 40 <gRNA; DNA; Artificial Sequence>
ATATCAATAATATAACCACCTAAGGGT
SEQ ID NO: 41 <gRNA; DNA; Artificial Sequence>
ATGCACAGATATAAACACTTAACGGGT
SEQ ID NO: 42 <gRNA; DNA; Artificial Sequence>
TGTTGGTGAAAAAATATAACTTTGAGT

### EXAMPLES

### Example 1

### Targeted Integration of DNA in the ATXN3 Gene

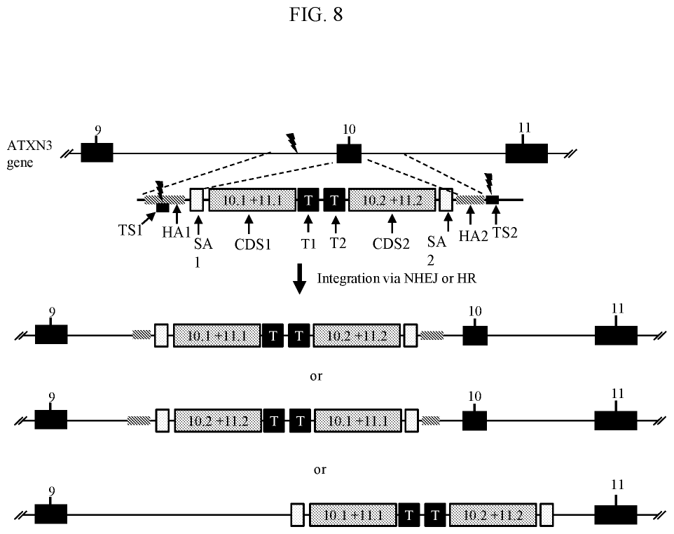

Three plasmids were constructed with transgenes designed to integrate into the ATXN3 gene in human cells. All transgenes were designed to be inserted within intron 9 or the junction of intron 9 and exon 10 of the ATXN3 gene and all transgenes were designed to insert at least one splice acceptor and at least one functional coding sequence for exons 10 and 11 of the ATXN3 gene. The first plasmid, designated pBA1135, comprised a left and right homology arm with sequence homologous to the 3′ end of intron 9 and 5′ end of intron 10 (i.e., successful gene targeting would result in removal of exon 10 and replacement with the cargo sequence within pBA1135). Between the homology arms, from 5′ to 3′, was a splice acceptor (splice acceptor from ATXN3 intron 9), coding sequence for exons 10 and 11 of ATXN3, SV40 terminator, reverse BGH terminator, reverse coding sequence for exons 10 and 11 (codon adjusted), and reverse splice acceptor (FIG. 8). The sequence for the pBA1135 transgene is shown in SEQ ID NO:1. A corresponding Cas9 nuclease was designed to cleave i) within intron 9 of the ATXN3 gene, ii) within the left homology arm of pBA1135, and iii) at the 3′ end of the right homology arm of pBA1135. Successful cleavage of the plasmid was expected to liberate the transgene, thereby enabling the sequence to be used as a template for HR or for integration via NHEJ. The Cas9 gRNA target site is shown in SEQ ID NO:2. The individual elements within pBA1135 are shown in SEQ ID NOS:3-10. SEQ ID NO:3 comprises the left homology arm, nuclease target site, and splice acceptor. SEQ ID NO:4 comprises the partial coding sequence (exon 10 and 11) of a non-pathogenic ATXN3 gene. SEQ ID NO:5 comprises the SV40 p(A) terminator sequence. SEQ ID NO:6 comprises the BGH terminator in reverse complement. SEQ ID NO:7 comprises the reverse complement, codon adjusted partial coding sequence (exon 10 and 11) of a non-pathogenic ATXN3 gene. SEQ ID NO:8 comprises the sequence for the splice acceptor. SEQ ID NO:9 comprises the sequence for the right homology arm. SEQ ID NO:10 comprises the target site sequence for the nuclease. The second plasmid, designated pBA1136, comprised the same cargo as pBA1135, however, the homology arms were removed. Nuclease target sites were kept to facilitate liberation of the transgene from the plasmid. Successful cleavage of the plasmid was expected to liberate the transgene, thereby enabling the sequence to be used for integration by NHEJ into the ATXN3 gene. The sequence of pBA1136 is shown in SEQ ID NO:11. The third plasmid, designated pBA1137, comprised the same sequence as pBA1135, except for the reverse sequences and nuclease target site (i.e., reverse terminator, reverse coding sequence and reverse splice acceptor). Plasmid pBA1137 was used as a control for conventional HR based methods. The sequence of pBA1137 is shown in SEQ ID NO:12.

Transfection was performed using HEK293T cells. HEK293T cells were maintained at 37° C. and 5% CO2 in DMEM high supplemented with 10% fetal bovine serum (FBS). HEK293T cells were transfected with 2 ug of donor, 2 ug of guide RNA (RNA format) and 2 ug of Cas9 (RNA format). Transfections were performed using electroporation. Genomic DNA was isolated 72 hours post transfection and assessed for integration events. A list of primers used to detect integration or genomic DNA is shown in Table 1.

TABLE 1
Primers for detecting integration of transgenes in
ATXN3.

| Primer | | |
|---|---|---|
| Name | Sequence (5′ to 3′) | SEQ ID NO: |
| oNJB043 | CAAAGGTGCCCTTGAGGTT | 13 |
| oNJB044 | AGGAGAAGTCTGCCGTTACT | 14 |
| oNJB113 | GGACAAACCACAACTAGAATGC | 15 |
| oNJB114 | TAGGAAAGGACAGTGGGAGT | 16 |
| oNJB116 | CCATTATGTCTCAGTTGTTCAGTG | 17 |
| oNJB156 | CCAGACCATCTCAGACACC | 18 |
| oNJB162 | GGCTGGGCTTCCACTTAC | 19 |
| oNJB167 | GTGGTTTGTCCAAACTCATCAA | 20 |
| oNJB170 | AGTAACTCTGCACTTCCCATTG | 21 |

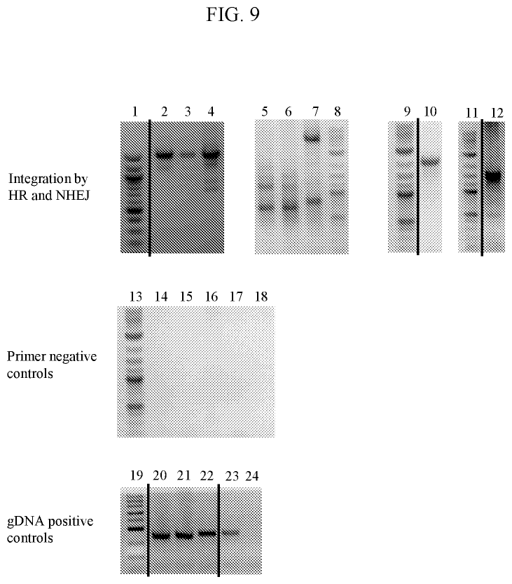

To detect the integration of pBA1135, pBA1136 and pBA1137, PCRs were performed on the genomic DNA. Regarding pBA1137, the transgene was designed to be integrated precisely by HR. Accordingly, bands were detected in the 5′ and 3′ junction PCRs, which indicate precise insertion into exon 10 (FIG. 9 lanes 4 and 7). Expected band sizes were 1,520 bp for the 5′ junction and 786 bp for the 3′ junction. Primers oNJB113 and oNJB116 were used for the 5′ junction PCR. Primers oNJB167 and oNJB170 were used for the 3′ junction PCR. Regarding pBA1136, as no homology arms were present, the transgene was predicted to insert via NHEJ insertion. Appropriate size bands were observed for the transgene integrating in the forward and reverse directions. Integration in the forward direction can be seen in FIG. 9 lanes 3 (expected size approximately 1,520 bp) and 6 (expected size approximately 1,519 bp). Integrating in the reverse direction can be seen in FIG. 9 lane 12 (expected size approximately 1,520 bp). Primers oNJB113 and oNJB116 were used for the 5′ junction PCR. Primers oNJB114 and oNJB170 were used for the 3′ junction PCR. Primers oNJB116 and oNJB114 were used for the inverse 5′ junction PCR. Regarding ppBA1135, both homology arms and nuclease cleavage sites were present on the transgene. Integration by HR was observed by detecting bands in the 5′ and 3′ junction PCRs (FIG. 9 lane 2 and 5). Further, integration by NHEJ was observed by detecting bands in an inverse 5′ junction PCR (FIG. 9 lane 10). Expected size for the 5′ junction PCR was 1,520 bp. Expected size for the 3′ junction PCR was 1,157 bp. Expected size for the inverse 5′ junction PCR was approximately 1,520 bp. Primers oNJB113 and oNJB116 were used for the 5′ junction PCR. Primers oNJB114 and oNJB170 were used for the 3′ junction PCR. Primers oNJB116 and oNJB114 were used for the inverse 5′ junction PCR.

The results show that the described transgenes comprising bidirectional coding sequences can be integrated into genomic DNA through multiple different repair pathways.

### Example 2

### Creating Cell Lines with Modified ATXN3 Alleles Comprising Corrective Coding Sequence that is Resistant to Silencing

A transgene comprising coding sequence encoding the peptide produced by exons 10 and 11 of the ATXN3 gene is generated. Following the coding sequence is a selectable marker for isolating cells with integration events. The transgene does not comprise the native ATXN3 3′ UTR. An siRNA is designed to silence endogenous ATXN3 alleles and targets the sequence CGUCGGUUGUAGGACUAAA (SEQ ID NO:22).

The transgene is integrated into the ATXN3 gene in HEK293 cells by lipofection. HEK293 cells are maintained at 37° C. and 5% CO2 in DMEM high glucose without L-glutamine without sodium pyruvate medium supplemented with 10% fetal bovine serum (FBS) and 1% penicillin-streptomycin (PS) solution 100X. HEK293 cells are transfected with Cas9 and the transgene comprising exons 10 and 11 of the ATXN3 gene. Transfections were performed using Lipofectamine 3000. Single cell clones are isolated and screened for integration events. Successful integration of the transgene is analyzed using PCR. Cell lines comprising the integrated transgene in one allele are subject to another round of transfection with the siRNA. Two days post transfection, RNA is extracted and assessed for levels of ATXN3 WT and modified mRNA.

### Example 3

### Creating Cell Lines with Modified SOD1 Alleles Comprising Corrective Coding Sequence that is Resistant to Silencing

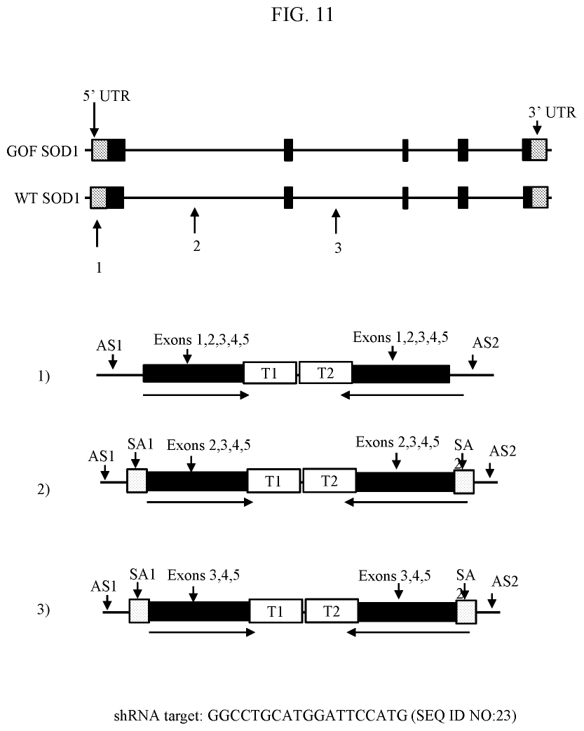

Transgenes comprising bidirectional coding sequence encoding the peptide produced by exons 1-5, 2-5 or 3-5 of the SOD1 gene are generated (FIG. 11). The coding sequences are operably linked to two terminators. The target site for integration is the 5′ UTR of the SOD1 gene. shRNA is designed to silence unmodified endogenous SOD1 alleles and targets the sequence GGCCUGCAUGGAUUCCAUG (SEQ ID NO:23). The corresponding SOD1 coding sequences within the transgene are modified with synonymous mutations to avoid silencing.

The transgene and Cas9 constructs targeting the SOD1 5′ UTR are transfected into N2A cells along with the shRNA targeting mRNA from endogenous SOD1 alleles. Three days post transfection DNA and RNA are isolated. The DNA is assessed by PCR for targeted insertion of the transgene into the SOD1 5′ UTR. The RNA is assessed for mRNA levels of endogenous SOD1, modified SOD1 comprising sequence from the first coding sequence, and modified SOD1 comprising sequence from the second coding sequence.

### Example 4

### Creating Cell Lines with Modified CACNA1A Alleles Comprising Corrective Coding Sequence that is Resistant to Silencing

A transgene comprising bidirectional coding sequence encoding the peptide produced by exons 47 of the CACNA1A gene is generated. The coding sequences are operably linked to two splice acceptors and two terminators. The target site for integration is intron 46 of the endogenous CACNA1A gene. shRNA is designed to silence unmodified endogenous CACNA1A alleles and targets sequence within the mRNA encoded by exon 47 of the CACNA1A gene. The corresponding CACNA1A coding sequences within the transgene are modified with synonymous mutations to avoid silencing.

The transgene and Cas9 constructs targeting the CACNA1A intron 46 are transfected into N2A cells along with the shRNA targeting mRNA from exon 47 of the endogenous CACNA1A gene. Three days post transfection DNA and RNA are isolated. The DNA is assessed by PCR for targeted insertion of the transgene into the CACNA1A 5′ UTR. The RNA is assessed for mRNA levels of endogenous CACNA1A, modified CACNA1A comprising sequence from the first coding sequence, and modified CACNA1A comprising sequence from the second coding sequence.

### Example 5

### Silencing and Expressing SERPINA1 In Vivo

A transgene comprising SERPINA1 coding sequence resistant to silencing is integrated into the albumin gene in mice livers. A second vector comprising an shRNA is delivered to mice livers following delivery of the first set of transgene.

The transgene comprises, from 5′ to 3′, an AAV2 ITR sequence, a mouse albumin intron 1 splice acceptor, a SERPINA1 coding sequence, an SV40 terminator, a BGH terminator (reverse complement), a SERPINA1 coding sequence (reverse complement and codon optimized), a human factor IX splice acceptor (reverse complement), and an AAV2 ITR sequence. The sequence for the transgene is provided in SEQ ID NO:26. The SERPINA1 coding sequences comprises synonymous mutations at the corresponding shRNA target site to prevent silencing. The transgene is packaged in AAV-DJ particles as single stranded DNA.

The second vector comprises AAV2 ITRs flanking an RNA polymerase III promoter (H1) driving expression of a short dsRNA in the form of an inverted repeat sequence containing a hairpin loop. The shRNA has the target sequence GAUGAAGCGUUUAGGCAUGUU (SEQ ID NO:27). The shRNA vector is packaged into AAV8 particles as a self-complementary vector.

All-in-one CRISPR vectors expressing SaCas9 and a gRNA and targeting sequence within albumin intron 1 are designed. Target sites for SaCas9 within intron 1 include: TTCAACTGTATCCAACGTAATTTGAGT (SEQ ID NO:33), CTCAAATTACGTTGGATACAGTTGAAT (SEQ ID NO:34), ACATGACAGAAACACTAAATCTTGAGT (SEQ ID NO:35), GATCGGGAACTGGCATCTTCAGGGAGT (SEQ ID NO:36), GGCAGAATGACTCAAATTACGTTGGAT (SEQ ID NO:37), GTATCTTTGATGACAATAATGGGGGAT (SEQ ID NO:38), CAGAAACACTAAATCTTGAGTTTGAAT (SEQ ID NO:39), ATATCAATAATATAACCACCTAAGGGT (SEQ ID NO:40), ATGCACAGATATAAACACTTAACGGGT (SEQ ID NO:41), TGTTGGTGAAAAAATATAACTTTGAGT (SEQ ID NO:42), GTGTGTTACTAATTTTATAAATGGAGT (SEQ ID NO:24), and TAATTAAAAAAAAATGCTAGGCAGAAT (SEQ ID NO:25). The vectors are packaged in AAV-DJ particles as single stranded DNA and co-delivered with the transgene.

C57BL mice are administered the AAV particles comprising the Cas9 and transgenes via tail-vein injection. Seven days following injection, the same mice are subsequently administered the AAV particles comprising the shRNA vector via tail-vein injection. Mice are sacrificed 21 days post injection of the first AAV particles. Blood is isolated and assessed for AAT levels via ELISA. Genomic DNA from the liver is isolated and assessed for targeted insertion of the transgene. Further, mRNA is extracted from the liver and assessed for i) expression of SERPINA mRNA comprising the silencing resistant mutations and ii) decreased expression of the endogenous SERPINA mRNA.

As an alternative to delivering the shRNA through an AAV vector, the shRNA or siRNA is purified as RNA and delivered to liver cells via lipid nanoparticles and tail vein injection. The lipid formulation includes the ionizable lipid ((9Z, 12Z)-3-((4,4-bis(octyloxy)butanoyl)oxy)-2-((((3-(diethylamino)propoxy)carbonyl)oxy)methyl)propyl octadeca-9,12-dienoate. The lipid formulation is also referred to as 3-((4,4-bis(octyloxy) butanoyl)oxy)-2-((((3-(diethylamino)propoxy)carbonyl)oxy)methyl)propyl(9Z,12Z)-octadeca-9,12-dienoate), cholesterol, DSPC, and PEG2k-DMG. The lipid nanoparticles comprising shRNA or siRNA targeting SERPINA are administered to mice seven days following injection of the gene editing agents. Mice are sacrificed 21 days post injection of the first AAV particles. Blood is isolated and assessed for AAT levels via ELISA. Genomic DNA from the liver is isolated and assessed for targeted insertion of the transgene. Further, mRNA is extracted from the liver and assessed for i) expression of SERPINA1 mRNA comprising the silencing resistant mutations and ii) decreased expression of the endogenous SERPINA1 mRNA.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of modifying expression of an endogenous gene in a cell comprising a first allele of the endogenous gene and a second allele of the endogenous gene, the method comprising:
a. administering a transgene to the cell, wherein the transgene comprises a first coding sequence that encodes an amino acid sequence that is homologous to the protein encoded by the endogenous gene or to a polypeptide fragment thereof,
b. integrating the transgene into the first allele of the endogenous gene to create a modified first allele, and
c. administering a silencing agent to the cell that reduces expression of the endogenous gene;
wherein the first coding sequence is not silenced by the silencing agent, wherein the modified first allele is expressed at a higher level than the second allele.

2. The method of claim 1, wherein the first coding sequence is operably linked to a first splice acceptor sequence.

3. The method of claim 2, wherein the first coding sequence is operably linked to a first terminator.

4. The method of claim 3, wherein the first coding sequence comprises synonymous mutations compared to a corresponding wild type sequence, wherein the synonymous mutations reduce silencing of the modified first allele by the silencing agent, or wherein the first coding sequence is not operably linked to a 5′ or 3′ UTR compared to the corresponding WT sequence.

5. The method of claim 4, wherein the transgene further comprises a second coding sequence and a second splice acceptor sequence.

6. The method of claim 5, wherein the second coding sequence is operably linked to the second splice acceptor sequence.

7. The method of claim 6, wherein the second coding sequence is operably linked to a second terminator, or wherein the first terminator is a bidirectional terminator and both the first coding sequence and the second coding sequence are operably linked to the bidirectional terminator.

8. The method of claim 7, wherein the first and second coding sequences are positioned in a tail-to-tail orientation.

9. The method of claim 8, wherein the first and second coding sequences comprise nucleotide differences compared to the corresponding wild-type sequence, wherein the nucleotide differences reduce silencing of the modified first allele by the silencing agent.

10. The method of claim 9, wherein the second coding sequence encodes an amino acid sequence that is homologous to the protein encoded by the endogenous gene or to a polypeptide fragment thereof

11. The method of claim 10, wherein the first and second coding sequences encode the same amino acid sequence.

12. The method of claim 11, wherein the first and second coding sequences differ in nucleic acid sequence.

13. The method of claim 1, wherein a viral vector comprising the transgene is administered.

14. The method of claim 13, wherein the viral vector is selected from the group consisting of an adenovirus vector, an adeno-associated virus vector, and a lentivirus vector.

15. The method of claim 14, wherein the transgene is equal to or less than 4.7 kb.

16. The method of claim 1, wherein a non-viral vector comprising the transgene is administered.

17. The method of claim 1, further comprising administering a rare-cutting endonuclease to the cell, wherein the rare-cutting endonuclease creates a double-stranded break within the endogenous gene.

18. The method of claim 17, wherein the double-stranded break occurs within an intron.

19. The method of claim 17, wherein the rare-cutting endonuclease is selected from a CRISPR nuclease, a TAL effector nuclease, a zinc-finger nuclease, and a meganuclease.

20. The method of claim 19, wherein a viral vector comprising nucleic acid encoding the rare-cutting endonuclease is administered to the cell.

21. The method of claim 19, wherein a rare-cutting endonuclease protein or nucleic acid is administered to the cell.

22. The method of claim 21, wherein the rare-cutting endonuclease is administered to the cell using lipid nanoparticles.

23. The method of claim 1, wherein the endogenous gene is selected from SERPINA1, SOD1, TRPV4, CHRNA1, CHRND, CHRNE, CHRNB1, PRPS1, LRRK2, STIM1, FGFR3, MECP2, SNCA, ATXN1, ATXN2, ATXN3, CACNA1A, ATXN7, TBP, HTT, AR, FXN, DMPK, PABPN1, ATXN8, RHO, and C9orf72.

24. The method of claim 1, wherein the silencing agent is selected from a DNA oligonucleotide agent and an RNA oligonucleotide agent.

25. The method of claim 24, wherein the DNA oligonucleotide agent is an antisense oligonucleotide.

26. The method of claim 24, wherein the RNA oligonucleotide agent is selected from a microRNA, a short hairpin RNA, a double-stranded RNA and a short interfering RNA.

27. The method of claim 26, wherein a viral vector comprising nucleic acid encoding the RNA oligonucleotide agent is administered to the cell.

28. The method of claim 26, wherein the RNA oligonucleotide agent is administered to the cell using lipid nanoparticles.

29. The method of claim 1, wherein the transgene does not encode the silencing agent.

30. The method of claim 29, wherein the silencing agent is administered after the transgene is administered.

31. The method of claim 1, wherein the second allele has about 10%, 20%, 30%, 40%, 50%, 60%, 70%, 80%, 90%, 95%, or 99% reduced expression compared to the expression of the second allele in a cell that is not administered a silencing agent.

32. The method of claim 1, wherein the second allele has about 5-10%, 10-20%, 20-30%, 30-40%, 40-50%, 50-60%, 60-70%, 70-80%, 80-90%, 90-95%, or 95-99% reduced expression compared to the expression of the second allele in a cell that is not administered a silencing agent.

33. The method of claim 1, wherein the cell comprises a gain-of-function mutation in one allele of the endogenous gene.

34. The method of claim 1, wherein the endogenous gene comprises more than two alleles.

35. The method of claim 34, wherein the endogenous gene is SNCA.

36. A method of modifying expression of a first endogenous gene in a cell, the method comprising:
a. administering a transgene to the cell, wherein the transgene comprises a first coding sequence that encodes an amino acid sequence that is homologous to an amino acid sequence encoded by the first endogenous gene,
b. integrating the transgene into a second endogenous gene in the cell,
c. administering a silencing agent to the cell that reduces expression of the first endogenous gene,
wherein the transgene comprises a coding sequence that is not silenced by the silencing agent, wherein the modified first allele is expressed at a higher level than the non-modified alleles.

37. The method of claim 36, wherein the t first coding sequence is operably linked to a first splice acceptor sequence.

38. The method of claim 37, wherein the first coding sequence is operably linked to a first terminator.

39. The method of claim 38, wherein the first coding sequence comprises synonymous mutations compared to the corresponding wild type sequence, wherein the synonymous mutations reduce silencing of the modified first allele by the silencing agent, or wherein the first coding sequence is not operably linked to a 5′ or 3′ UTR compared to the corresponding WT sequence.

40. The method of claim 39, wherein the transgene further comprises a second coding sequence and a second splice acceptor sequence.

41. The method of claim 40, wherein the second coding sequence is operably linked to the second splice acceptor sequence.

42. The method of claim 41, wherein the second coding sequence is operably linked to a second terminator, or wherein the first terminator is a bidirectional terminator and both the first coding sequence and the second coding sequence are operably linked to the bidirectional terminator.

43. The method of claim 42, wherein the transgene further comprises a second coding sequence and a second splice acceptor sequence

44. The method of claim 43, wherein the first and second coding sequences are positioned in a tail-to-tail orientation.

45. The method of claim 44, wherein the first and second coding sequences comprise nucleotide differences compared to the corresponding wild type sequence, wherein the nucleotide differences reduce silencing of the modified first allele by the silencing agent.

46. The method of claim 45, wherein the second coding sequence encodes an amino acid sequence that is homologous to an amino acid sequence encoded by the endogenous gene.

47. The method of claim 46, wherein the first and second coding sequences encode the same amino acid sequence.

48. The method of claim 47, wherein the first and second coding sequences differ in nucleic acid sequence.

49. The method of claim 37, wherein a viral vector comprising the transgene is administered.

50. The method of claim 49, wherein the viral vector is selected from the group consisting of an adenovirus vector, an adeno-associated virus vector, and a lentivirus vector.

51. The method of claim 49, wherein the transgene is equal to or less than 4.7 kb.

52. The method of claim 51, wherein a non-viral vector comprising the transgene is administered.

53. The method of claim 52, further comprising administering a rare-cutting endonuclease to the cell, wherein the rare-cutting endonuclease creates a double-stranded break within the second endogenous gene.

54. The method of claim 53, wherein the double-stranded break occurs within an intron.

55. The method of claim 53, wherein the rare-cutting endonuclease is selected from a CRISPR nuclease, a TAL effector nuclease, a zinc-finger nuclease, and a meganuclease.

56. The method of claim 55, wherein a viral vector comprising nucleic acid encoding the rare-cutting endonuclease is administered to the cell.

57. The method of claim 55, wherein a rare-cutting endonuclease protein or nucleic acid is administered to the cell.

58. The method of claim 57, wherein the rare-cutting endonuclease is administered to the cell using lipid nanoparticles.

59. The method of claim 36, wherein the silencing agent is selected from a DNA oligonucleotide agent and an RNA oligonucleotide agent.

60. The method of claim 59, wherein the DNA oligonucleotide agent is an antisense oligonucleotide.

61. The method of claim 59, wherein the RNA oligonucleotide agent is selected from a microRNA, a short hairpin RNA, a double-stranded RNA and a short interfering RNA.

62. The method of claim 61, wherein a viral vector comprising nucleic acid encoding the RNA oligonucleotide agent is administered to the cell.

63. The method of claim 61, wherein the RNA oligonucleotide agent is administered to the cell using lipid nanoparticles.

64. The method of claim 36, wherein the transgene does not encode the silencing agent.

65. The method of claim 64, wherein the silencing agent is administered after the transgene is administered.

66. The method of claim 36, wherein the first endogenous gene has about 10%, 20%, 30%, 40%, 50%, 60%, 70%, 80%, 90%, 95%, or 99% reduced expression compared to the expression of the first endogenous gene in a cell that is not administered a silencing agent.

67. The method of claim 36, wherein the first endogenous gene has about 5-10%, 10-20%, 20-30%, 30-40%, 40-50%, 50-60%, 60-70%, 70-80%, 80-90%, 90-95%, or 95-99% reduced expression compared to the expression of the first endogenous gene in a cell that is not administered a silencing agent.

68. The method of claim 36, wherein the first endogenous gene is SERPINA1 and the second endogenous gene is albumin.

69. A method for treating alpha-1 antitrypsin deficiency in a subject, wherein the subject comprises cells with a first and second SERPINA1 allele, and an albumin gene, the method comprising:
a. administering a transgene, wherein the transgene comprises a SERPINA1 coding sequence,
b. integrating the transgene into an intron within an endogenous albumin gene, and
c. administering a silencing agent that reduces expression of the endogenous SERPINA1 alleles, but not the SERPINA1 sequence within the transgene.

70. The method of claim 69, wherein the silencing agent is selected from a DNA oligonucleotide agent or an RNA oligonucleotide agent.

71. The method of claim 70, wherein the DNA oligonucleotide agent is antisense oligonucleotides.

72. The method of claim 70, wherein the RNA oligonucleotide agent is microRNA, short hairpin RNA, double-stranded RNA or short interfering RNA.

73. The method of claim 72, wherein a viral vector comprising nucleic acid encoding the RNA oligonucleotide agent is administered to the cell.

74. The method of claim 72, wherein the RNA oligonucleotide agent is administered to the cell using lipid nanoparticles.

75. The method of claim 69, wherein the SERPINA1 coding sequence comprises synonymous mutations at the corresponding target site of the silencing sequence.

76. The method of claim 69, wherein the transgene comprises, in 5′ to 3′ orientation, a first splice acceptor, a first SERPINA1 coding sequence, a first terminator, a second terminator reverse complement, a second SERPINA1 coding sequence reverse complement, and a second splice acceptor reverse complement.

77. The method of claim 76, wherein the first and second SERPINA1 coding sequence comprises synonymous mutations at the corresponding target site of the silencing sequence.

78. The method of claim 69, wherein the transgene is integrated into albumin using a rare-cutting endonuclease.

79. The method of claim 78, wherein the rare-cutting endonuclease is selected from a CRISPR nuclease, a zinc-finger nuclease, a TAL effector nuclease, or a meganuclease.

80. The method of claim 79, wherein the CRISPR nuclease is a CRISPR/Cas9 nuclease.

81. The method of claim 78, wherein the transgene is integrated into intron 1 or intron 13 of the albumin gene.
